(19) United States
(12) Patent Application Publication
(10) Pub. No.: US 2022/0098158 A1
(43) Pub. Date: Mar. 31, 2022

(54) COLORING COMPOSITION, FILM, COLOR FILTER, AND SOLID-STATE IMAGING ELEMENT

(71) Applicant: FUJIFILM Corporation, Tokyo (JP)

(72) Inventors: Yasuhiro SAWAMURA, Shizuoka (JP); Yushi KANEKO, Shizuoka (JP)

(73) Assignee: FUJIFILM Corporation, Tokyo (JP)

(21) Appl. No.: 17/544,942

(22) Filed: Dec. 8, 2021

Related U.S. Application Data
(63) Continuation of application No. PCT/JP2020/024479, filed on Jun. 23, 2020.

(30) Foreign Application Priority Data
Jul. 3, 2019 (JP) 2019-124643

(51) Int. Cl. C07D 235/26; G03F 7/00; C07D 403/12; C08F 220/26; G02B 5/20; H01L 27/146

(52) U.S. Cl. CPC C07D 235/26; G03F 7/0007; H01L 27/14621; C08F 220/26; G02B 5/20; C07D 403/12

(57) ABSTRACT

Provided are a coloring composition for a solid-state imaging element, including a pigment, a compound having a triazine ring, a polymerizable compound, and a photopolymerization initiator, in which the pigment is contained in an amount of 50% by mass or more in a total solid content of the coloring composition, and the compound having a triazine ring includes at least one group selected from an acid group or a basic group, and a maximum value of a molar absorption coefficient of the compound having a triazine ring in a wavelength range of 400 to 700 nm is 3000 L·mol⁻¹·cm⁻¹ or less; a film using the coloring composition; a color filter; and a solid-state imaging element.

### CROSS-REFERENCE TO RELATED APPLICATIONS

This application is a Continuation of PCT International Application No. PCT/JP2020/024479 filed on Jun. 23, 2020, which claims priority under 35 U.S.C § 119(a) to Japanese Patent Application No. 2019-124643 filed on Jul. 3, 2019. Each of the above application(s) is hereby expressly incorporated by reference, in its entirety, into the present application.

### BACKGROUND OF THE INVENTION

### 1. Field of the Invention

The present invention relates to a coloring composition including a pigment. The present invention further relates to a film using the coloring composition, a color filter, and a solid-state imaging element.

### 2. Description of the Related Art

In recent years, as a digital camera, a mobile phone with a camera, and the like have been further spreading, there has been a greatly increasing demand for a solid-state imaging element such as a charge coupled device (CCD) image sensor. A color filter has been used as a key device in a display or an optical element.

The color filter has been produced using a coloring composition including a colorant and a polymerizable compound. In addition, in a case where a pigment is used as the colorant, the pigment is generally dispersed in the coloring composition using a dispersant or the like.

JP2003-081972A discloses an invention relating to a coloring composition which includes a pigment dispersion composition prepared by dispersing a predetermined triazine compound and a pigment in an organic solvent, a binder polymer having an acid group, a polyfunctional monomer having two or more ethylenically unsaturated double bonds, and a photopolymerization initiator.

JP2005-173287A discloses an invention relating to a coloring composition for a color filter, which includes a pigment carrier, an isoindoline pigment, an azo pigment, a phthalocyanine pigment, a triazine derivative having a basic group, and the like.

### SUMMARY OF THE INVENTION

In recent years, there has been a strong demand for miniaturization and film-thinning in a solid-state imaging element. Therefore, in recent years, it has been desired to further reduce a thickness of a film including a pigment, such as a color filter, used in the solid-state imaging element.

In order to achieve a thin film while maintaining desired spectral performance, it is necessary to increase a concentration of the pigment in a coloring composition used for film formation.

However, in a case where the concentration of the pigment in the coloring composition is increased, since the content of components other than the pigment is relatively small, dispersibility of the pigment in the coloring composition tends to decrease. Therefore, viscosity of the coloring composition tends to increase with time, and there is room for improvement in storage stability of the coloring composition. In addition, in a case where the concentration of the pigment in the coloring composition is increased, curing properties of the coloring composition tends to decrease, and there is also room for further improvement in adhesiveness of the film formed of the coloring composition with a support.

Therefore, an object of the present invention is to provide a coloring composition capable of forming a film having excellent storage stability and excellent adhesiveness with a support. Another object of the present invention is to provide a film using the coloring composition, a color filter, and a solid-state imaging element.

According to the studies conducted by the present inventors, it has been found that the above-described object can be achieved by adopting the following configuration, thereby leading to the completion of the present invention. Therefore, the present invention provides the following.

<1> A coloring composition for a solid-state imaging element, comprising:

a pigment;

a compound having a triazine ring;

a polymerizable compound; and

a photopolymerization initiator,

in which the pigment is contained in an amount of 50% by mass or more in a total solid content of the coloring composition, and

the compound having a triazine ring includes at least one group selected from an acid group or a basic group, and a maximum value of a molar absorption coefficient of the compound having a triazine ring in a wavelength range of 400 to 700 nm is 3000 L·mol⁻¹·cm⁻¹ or less.

<2> The coloring composition according to <1>,

in which the compound having a triazine ring includes two or more triazine rings in one molecule.

<3> The coloring composition according to <1> or <2>,

in which the compound having a triazine ring includes a group represented by Formula (C1),

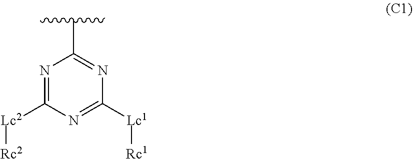

in the formula, a wavy line represents a bonding site, Lc¹ and Lc² each independently represent a single bond or a linking group, Rc¹ and Rc² each independently represent a substituent, and at least one of Rc¹ or Rc² represents an acid group or a basic group.

<4> The coloring composition according to <3>,

in which the group represented by Formula (C1) is a group represented by Formula (C2),

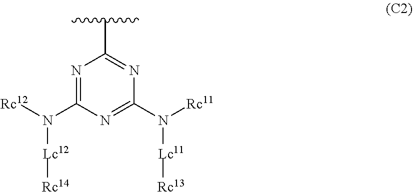

in the formula, a wavy line represents a bonding site, Lc¹¹ and Lc¹² each independently represent a single bond or a linking group, Rc¹¹ and Rc¹² each independently represent a hydrogen atom or a substituent, Rc¹³ and Rc¹⁴ each independently represent a substituent, and at least one of Rc¹³ or Rc' represents an acid group or a basic group.

<5> The coloring composition according to <3>,

in which the group represented by Formula (C1) is a group represented by Formula (C3),

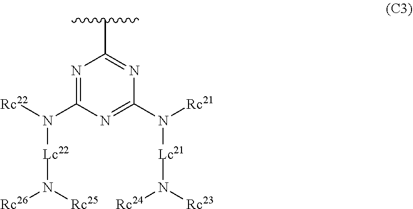

in the formula, a wavy line represents a bonding site, Lc²¹ and Lc²² each independently represent a single bond or a linking group, Rc²¹ and Rc²² each independently represent a hydrogen atom or a substituent, Rc²³ to Rc²⁶ each independently represent a hydrogen atom or a substituent, Rc²³ and Rc²⁴ may be bonded to each other through a divalent group to form a ring, and Rc²⁵ and Rc²⁶ may be bonded to each other through a divalent group to form a ring.

<6> The coloring composition according to any one of <1> to <5>,

in which the pigment is an organic pigment.

<7> The coloring composition according to any one of <1> to <6>,

in which the pigment is a chromatic pigment.

<8> The coloring composition according to any one of <1> to <7>,

in which the pigment includes a phthalocyanine pigment.

<9> The coloring composition according to any one of <1> to <8>,

in which the pigment includes a green pigment.

<10> The coloring composition according to any one of <1> to <9>,

in which the photopolymerization initiator includes an oxime compound.

<11> The coloring composition according to any one of <1> to <10>, further comprising:

an alkali-soluble resin.

<12> The coloring composition according to any one of <1> to <11>, further comprising:

a resin having an aromatic carboxyl group.

<13> A film obtained from the coloring composition according to any one of <1> to <12>.

<14> A color filter comprising:

the film according to <13>.

<15> A solid-state imaging element comprising:

the film according to <13>.

According to the present invention, it is possible to provide a coloring composition capable of forming a film having excellent storage stability and excellent adhesiveness with a support. It is also possible to provide a film using the coloring composition, a color filter, and a solid-state imaging element.

### DESCRIPTION OF THE PREFERRED EMBODIMENTS

Hereinafter, the contents of the present invention will be described in detail.

In the present specification, “to” is used to refer to a meaning including numerical values denoted before and after “to” as a lower limit value and an upper limit value.

In the present specification, unless specified as a substituted group or as an unsubstituted group, a group (atomic group) denotes not only a group (atomic group) having no substituent but also a group (atomic group) having a substituent. For example, an “alkyl group” includes not only an alkyl group having no substituent (unsubstituted alkyl group), but also an alkyl group having a substituent (substituted alkyl group).

In the present specification, unless specified otherwise, “exposure” denotes not only exposure using light but also drawing using a corpuscular beam such as an electron beam or an ion beam. In addition, examples of light used for the exposure include actinic rays or radiation such as a bright line spectrum of a mercury lamp, far ultraviolet rays typified by an excimer laser, extreme ultraviolet rays (EUV light), X-rays, or electron beams.

In the present specification, “(meth)acrylate” denotes either or both of acrylate and methacrylate, “(meth)acryl” denotes either or both of acryl and methacryl, and “(meth)acryloyl” denotes either or both of acryloyl and methacryloyl.

In the present specification, in structural formulae, Me represents a methyl group, Et represents an ethyl group, Bu represents a butyl group, and Ph represents a phenyl group.

In the present specification, a weight-average molecular weight and a number-average molecular weight are values in terms of polystyrene through measurement by a gel permeation chromatography (GPC) method.

In the present specification, near-infrared rays denote light having a wavelength in a range of 700 to 2500 nm.

In the present specification, the total solid content refers to a total mass of components other than a solvent from all the components of a composition.

In the present specification, a pigment means a compound which is hardly dissolved in a solvent. For example, as the pigment, both of the solubility in 100 g of water at 23° C. and 100 g of propylene glycol monomethyl ether acetate at 23° C. is preferably 0.1 g or less and more preferably 0.01 g or less.

In the present specification, the term “step” is not only an independent step, but also includes a step which is not clearly distinguished from other steps in a case where an intended action of the step is obtained.

<Coloring Composition>

A coloring composition according to an embodiment of the present invention is a coloring composition for a solid-state imaging element, including a pigment, a compound having a triazine ring, a polymerizable compound, and a photopolymerization initiator, in which the pigment is contained in an amount of 50% by mass or more in a total solid content of the coloring composition, and the compound having a triazine ring includes at least one group selected from an acid group or a basic group, and a maximum value of a molar absorption coefficient of the compound having a triazine ring in a wavelength range of 400 to 700 nm is 3000 L·mol⁻¹·cm⁻¹ or less.

<Coloring Composition>

The coloring composition according to the embodiment of the present invention is a coloring composition for a solid-state imaging element, including a pigment, a compound having a triazine ring, a polymerizable compound, and a photopolymerization initiator, in which the pigment is contained in an amount of 50% by mass or more in a total solid content of the coloring composition, and the compound having a triazine ring includes at least one group selected from an acid group or a basic group, and a maximum value of a molar absorption coefficient of the compound having a triazine ring in a wavelength range of 400 to 700 nm is 3000 L·mol⁻¹·cm⁻¹ or less.

Hereinafter, a compound that is a compound having a triazine ring, which includes at least one group selected from an acid group or a basic group, and in which a maximum value of a molar absorption coefficient of the compound in a wavelength range of 400 to 700 nm is 3000 L·mol⁻¹·cm⁻¹ or less, is also referred to as a triazine compound (TA).

Since the coloring composition according to the embodiment of the present invention includes the above-described triazine compound (TA), dispersibility of the pigment in the coloring composition can be improved, and the coloring composition can have excellent storage stability. In addition, with the coloring composition according to the embodiment of the present invention, it is possible to form a film having excellent adhesiveness with a support. It is presumed that the reason why a film having excellent adhesiveness with a support can be formed is as follows. That is, since the above-described triazine compound (TA) has a small molar absorption coefficient at a wavelength of 400 to 700 nm, in a case where the coloring composition is applied to a support and exposed with light to form a film, it is presumed that the light can be transmitted to a deep portion of the film (a support side of the film) by the exposure. As a result, it is presumed that, even in the deep portion of the film, the photopolymerization initiator can be decomposed to sufficiently generate active species such as radicals, and it is possible to sufficiently cure the film to the deep portion of the film. In addition, it is presumed that a strong network is formed between the pigment and the triazine compound (TA) in the film, and it is presumed that the formation of such a network makes the film stronger. Furthermore, it is presumed that an interaction occurs between the triazine ring of the triazine compound (TA) and the support, and the adhesiveness of the obtained film with the support is improved. With these synergistic effects, it is presumed that it is possible to form a film having excellent adhesiveness with a support.

According to the coloring composition according to the embodiment of the present invention, it is possible to form a film having a small spectral variation due to heating and having excellent heat resistance. It is presumed that the reason why such an effect is obtained is that the film is sufficiently cured by the same mechanism as described above.

The coloring composition according to the embodiment of the present invention can be used for a color filter, a near-infrared transmitting filter, a near-infrared cut filter, and the like.

Examples of the color filter include a filter having a colored pixel which transmits light having a specific wavelength, and a filter having at least one colored pixel selected from a red pixel, a blue pixel, a green pixel, a yellow pixel, a cyan pixel, or a magenta pixel is preferable. The color filter can be formed using a coloring composition including a chromatic pigment.

Examples of the near-infrared cut filter include a filter having a maximal absorption wavelength in a wavelength range of 700 to 1800 nm. As the near-infrared cut filter, a filter having a maximal absorption wavelength in a wavelength range of 700 to 1300 nm is preferable, and a filter having a maximal absorption wavelength in a wavelength range of 700 to 1000 nm is more preferable. In addition, in the near-infrared cut filter, a transmittance of in the entire wavelength range of 400 to 650 nm is preferably 70% or more, more preferably 80% or more, and still more preferably 90% or more. In addition, the transmittance at at least one point in a wavelength range of 700 to 1800 nm is preferably 20% or less. In addition, in the near-infrared cut filter, absorbance Aₘₐₓ/absorbance A₅₅₀, which is a ratio of an absorbance Amax at a maximal absorption wavelength to an absorbance A550 at a wavelength of 550 nm, is preferably 20 to 500, more preferably 50 to 500, still more preferably 70 to 450, and particularly preferably 100 to 400. The near-infrared cut filter can be formed using a coloring composition including a near-infrared absorbing pigment.

The near-infrared transmitting filter is a filter which transmits at least a part of near-infrared rays. The near-infrared transmitting filter may be a filter (transparent film) which transmits both visible light and near-infrared ray, or may be a filter which shields at least a part of visible light and transmits at least a part of near-infrared rays. Preferred examples of the near-infrared transmitting filter include filters satisfying spectral characteristics in which the maximum value of a transmittance in a wavelength range of 400 to 640 nm is 20% or less (preferably 15% or less and more preferably 10% or less) and the minimum value of a transmittance in a wavelength range of 1100 to 1300 nm is 70% or more (preferably 75% or more and more preferably 80% or more). The near-infrared transmitting filter is preferably a filter which satisfies any one of the following spectral characteristics (1) to (4).

(1): filter in which the maximum value of a transmittance in a wavelength range of 400 to 640 nm is 20% or less (preferably 15% or less and more preferably 10% or less) and the minimum value of a transmittance in a wavelength range of 800 to 1300 nm is 70% or more (preferably 75% or more and more preferably 80% or more).

(2): filter in which the maximum value of a transmittance in a wavelength range of 400 to 750 nm is 20% or less (preferably 15% or less and more preferably 10% or less) and the minimum value of a transmittance in a wavelength range of 900 to 1300 nm is 70% or more (preferably 75% or more and more preferably 80% or more).

(3): filter in which the maximum value of a transmittance in a wavelength range of 400 to 830 nm is 20% or less (preferably 15% or less and more preferably 10% or less) and the minimum value of a transmittance in a wavelength range of 1000 to 1300 nm is 70% or more (preferably 75% or more and more preferably 80% or more).

(4): filter in which the maximum value of a transmittance in a wavelength range of 400 to 950 nm is 20% or less (preferably 15% or less and more preferably 10% or less) and the minimum value of a transmittance in a wavelength range of 1100 to 1300 nm is 70% or more (preferably 75% or more and more preferably 80% or more).

The coloring composition according to the embodiment of the present invention can be preferably used as a coloring composition for a color filter. Specifically, the coloring composition according to the embodiment of the present invention can be preferably used as a coloring composition for forming a pixel of a color filter, and can be more preferably used as a coloring composition for forming a green pixel of a color filter. In addition, the coloring composition according to the embodiment of the present invention can also be suitably used for the solid-state imaging element having a pixel configuration described in WO2019/102887A.

The concentration of solid contents of the coloring composition according to the embodiment of the present invention is preferably 5% to 40% by mass. The lower limit is preferably 10% by mass or more and more preferably 12% by mass or more. The upper limit is preferably 35% by mass or less and more preferably 30% by mass or less.

Hereinafter, the respective components used in the coloring composition according to the embodiment of the present invention will be described.

<<Pigment>>

The coloring composition according to the embodiment of the present invention contains a pigment. Examples of the pigment include a white pigment, a black pigment, a chromatic pigment, and a near-infrared absorbing pigment. In the present invention, the white pigment includes not only a pure white pigment but also a bright gray (for example, grayish-white, light gray, and the like) pigment close to white. In addition, the pigment may be an inorganic pigment or an organic pigment, but from the viewpoint that dispersion stability is more easily improved, an organic pigment is preferable. In addition, as the pigment, a pigment having a maximal absorption wavelength in a wavelength range of 400 to 2000 nm is preferable, and a pigment having a maximal absorption wavelength in a wavelength range of 400 to 700 nm is more preferable. In addition, in a case of using a pigment (preferably a chromatic pigment) having a maximal absorption wavelength in a wavelength range of 400 to 700 nm, the coloring composition according to the embodiment of the present invention can be preferably used as a coloring composition for forming a colored pixel in a color filter. Examples of the colored pixel include a red pixel, a green pixel, a blue pixel, a magenta pixel, a cyan pixel, and a yellow pixel.

It is preferable that the pigment includes at least one selected from a phthalocyanine pigment, a squarylium pigment, a dioxazine pigment, a quinacridone pigment, an anthraquinone pigment, a perylene pigment, an azo pigment, a diketopyrrolopyrrole pigment, an isoindoline pigment, or a quinophthalone pigment, it is more preferable to include at least one selected from a phthalocyanine pigment, a squarylium pigment, or a diketopyrrolopyrrole pigment, and from the reason that the effects of the present invention are more easily obtained, it is still more preferable to include a phthalocyanine pigment. In addition, the proportion of the phthalocyanine pigment in the total amount of the pigment included in the coloring composition is preferably 50% to 100% by mass, more preferably 60% to 100% by mass, and still more preferably 65% to 100% by mass.

In addition, it is also preferable that the pigment includes a green pigment. Generally, a film formed of a coloring composition including a green pigment tends to have low adhesiveness with a support, but according to the coloring composition according to the embodiment of the present invention, even the coloring composition including a green pigment can form a film having excellent adhesiveness with a support, so that the effects of the present invention are more remarkable. Examples of the green pigment include a phthalocyanine pigment and a squarylium pigment, and from the reason that storage stability is good, a phthalocyanine pigment is preferable.

The average primary particle diameter of the pigment is preferably 1 to 200 nm. The lower limit is preferably 5 nm or more and more preferably 10 nm or more. The upper limit is preferably 180 nm or less, more preferably 150 nm or less, and still more preferably 100 nm or less. In a case where the average primary particle diameter of the pigment is within the above-described range, dispersion stability of the pigment in the coloring composition is good. In the present invention, the primary particle diameter of the pigment can be determined from an image obtained by observing primary particles of the pigment using a transmission electron microscope. Specifically, a projected area of the primary particles of the pigment is determined, and the corresponding equivalent circle diameter is calculated as the primary particle diameter of the pigment. In addition, the average primary particle diameter in the present invention is the arithmetic average of the primary particle diameters with respect to 400 primary particles of the pigment. In addition, the primary particle of the pigment refers to a particle which is independent without aggregation.

(Chromatic Pigment)

The chromatic pigment is not particularly limited, and a known chromatic pigment can be used. Examples of the chromatic pigment include a pigment having a maximal absorption wavelength in a wavelength range of 400 to 700 nm. Examples thereof include a yellow pigment, an orange pigment, a red pigment, a green pigment, a violet pigment, and a blue pigment. Specific examples of these pigments include the following pigments. Color Index (C. I.) Pigment Yellow 1, 2, 3, 4, 5, 6, 10, 11, 12, 13, 14, 15, 16, 17, 18, 20, 24, 31, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, 214, 215, 228, 231, 232 (methine-based), 233 (quinoline-based), and the like (all of which are yellow pigments);

C. I. Pigment Orange 2, 5, 13, 16, 17:1, 31, 34, 36, 38, 43, 46, 48, 49, 51, 52, 55, 59, 60, 61, 62, 64, 71, and 73 (all of which are orange pigments);

C. I. Pigment Red 1, 2, 3, 4, 5, 6, 7, 9, 10, 14, 17, 22, 23, 31, 38, 41, 48:1, 48:2, 48:3, 48:4, 49, 49:1, 49:2, 52:1, 52:2, 53:1, 57:1, 60:1, 63:1, 66, 67, 81:1, 81:2, 81:3, 83, 88, 90, 105, 112, 119, 122, 123, 144, 146, 149, 150, 155, 166, 168, 169, 170, 171, 172, 175, 176, 177, 178, 179, 184, 185, 187, 188, 190, 200, 202, 206, 207, 208, 209, 210, 216, 220, 224, 226, 242, 246, 254, 255, 264, 270, 272, 279, 294 (xanthene-based, Organo Ultramarine, Bluish Red), 295 (monoazo-based), 296 (diazo-based), and the like (all of which are red pigments);

C. I. Pigment Green 7, 10, 36, 37, 58, 59, 62, and 63 (all of which are green pigments);

C. I. Pigment Violet 1, 19, 23, 27, 32, 37, 42, 60 (triarylmethane-based), 61 (xanthene-based), and the like (all of which are violet pigments); and

C. I. Pigment Blue 1, 2, 15, 15:1, 15:2, 15:3, 15:4, 15:6, 16, 22, 29, 60, 64, 66, 79, 80, 87 (monoazo-based), 88 (methine-based), and the like (all of which are blue pigments).

In addition, as the green pigment, a halogenated zinc phthalocyanine pigment having an average number of halogen atoms in one molecule of 10 to 14, an average number of bromine atoms in one molecule of 8 to 12, and an average number of chlorine atoms in one molecule of 2 to 5 can also be used. Specific examples thereof include the compounds described in WO2015/118720A. In addition, as the green pigment, a compound described in CN2010-6909027A, a phthalocyanine compound described in WO2012/102395A, which has phosphoric acid ester as a ligand, a phthalocyanine compound described in JP2019-008014A, a phthalocyanine compound described in JP2018-180023A, a compound described in JP2019-038958A, and the like can also be used.

In addition, an aluminum phthalocyanine compound having a phosphorus atom can also be used as the blue pigment. Specific examples thereof include the compounds described in paragraphs 0022 to 0030 of JP2012-247591A and paragraph 0047 of JP2011-157478A.

In addition, as the yellow pigment, compounds described in JP2017-201003A, compounds described in JP2017-197719A, compounds described in paragraph Nos. 0011 to “0062 and 0137 to 0276 of JP2017-171912A, compounds described in paragraph Nos. 0010 to 0062 and 0138 to 0295 of JP2017-171913A, compounds described in paragraph Nos. 0011 to 0062 and 0139 to 0190 of JP2017-171914A, compounds described in paragraph Nos. 0010 to 0065 and 0142 to 0222 of JP2017-171915A, quinophthalone compounds described in paragraph Nos. 0011 to 0034 of JP2013-054339A, quinophthalone compounds described in paragraph Nos. 0013 to 0058 of JP2014-026228A, isoindoline compounds described JP2018-062644A, quinophthalone compounds described in JP2018-203798A, quinophthalone compounds described in JP2018-062578A, quinophthalone compounds described in JP6432077B, quinophthalone compounds described in JP6432076B, quinophthalone compounds described in JP2018-155881A, quinophthalone compounds described in JP2018-111757A, quinophthalone compounds described in JP2018-040835A, quinophthalone compounds described in JP2017-197640A, quinophthalone compounds described in JP2016-145282A, quinophthalone compounds described in JP2014-085565A, quinophthalone compounds described in JP2014-021139A, quinophthalone compounds described in JP2013-209614A, quinophthalone compounds described in JP2013-209435A, quinophthalone compounds described in JP2013-181015A, quinophthalone compounds described in JP2013-061622A, quinophthalone compounds described in JP2013-054339A, quinophthalone compounds described in JP2013-032486A, quinophthalone compounds described in JP2012-226110A, quinophthalone compounds described in JP2008-074987A, quinophthalone compounds described in JP2008-081565A, quinophthalone compounds described in JP2008-074986A, quinophthalone compounds described in JP2008-074985A, quinophthalone compounds described in JP2008-050420A, quinophthalone compounds described in JP2008-031281A, quinophthalone compounds described in JP1973-032765A (JP-S48-032765A), quinophthalone compounds described in JP2019-008014A, a compound represented by Formula (QP1), and a compound represented by Formula (QP2) can also be used.

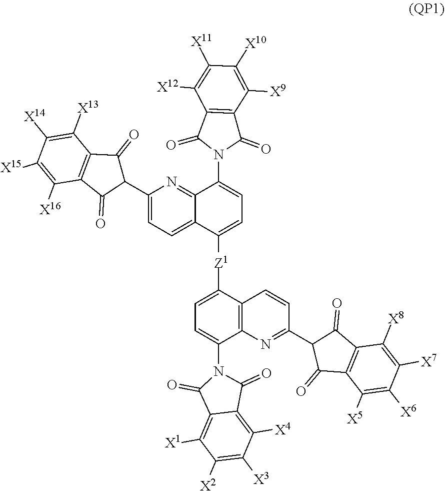

In Formula (QP1), X¹ to X¹⁶ each independently represent a hydrogen atom or a halogen atom, and Z¹ represents an alkylene group having 1 to 3 carbon atoms. Specific examples of the compound represented by Formula (QP1) include compounds described in paragraph No. 0016 of JP6443711B.

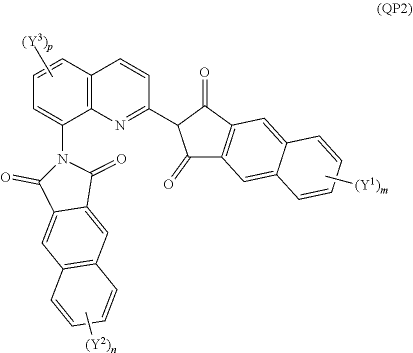

In Formula (QP2), Y¹ to Y³ each independently represent a halogen atom. n and m represent an integer of 0 to 6, and p represents an integer of 0 to 5. (n+m) is 1 or more. Specific examples of the compound represented by Formula (QP2) include compounds described in paragraph Nos. 0047 and 0048 of JP6432077B.

A diketopyrrolopyrrole pigment described in JP2017-201384A, in which the structure has at least one substituted bromine atom, a diketopyrrolopyrrole pigment described in paragraph Nos. 0016 to 0022 of JP6248838B, a red pigment described in JP6516119B, a red pigment described in JP6525101B, and the like can also be used as the red pigment. In addition, as the red pigment, a compound having a structure that an aromatic ring group in which a group bonded with an oxygen atom, a sulfur atom, or a nitrogen atom is introduced to an aromatic ring is bonded to a diketopyrrolopyrrole skeleton can be used. As the compound, a compound represented by Formula (DPP1) is preferable, and a compound represented by Formula (DPP2) is more preferable.

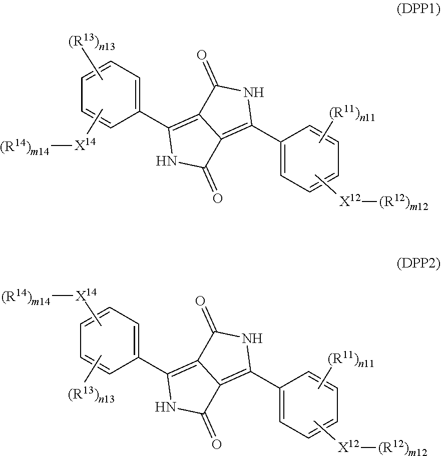

In the formulae, R¹¹ and R¹³ each independently represent a substituent, R¹² and R¹⁴ each independently represent a hydrogen atom, an alkyl group, an aryl group, or a heteroaryl group, n11 and n13 each independently represent an integer of 0 to 4, X¹² and X¹⁴ each independently represent an oxygen atom, a sulfur atom, or a nitrogen atom, in a case where X¹² is an oxygen atom or a sulfur atom, m12 represents 1, in a case where X¹² is a nitrogen atom, m12 represents 2, in a case where X¹⁴ is an oxygen atom or a sulfur atom, m14 represents 1, and in a case where X¹⁴ is a nitrogen atom, m14 represents 2. Examples of the substituent represented by R¹¹ and R¹³ include groups mentioned in the substituent T described later, and preferred specific examples thereof include an alkyl group, an aryl group, a halogen atom, an acyl group, an alkoxycarbonyl group, an aryloxycarbonyl group, a heteroaryloxycarbonyl group, an amide group, a cyano group, a nitro group, a trifluoromethyl group, a sulfoxide group, and a sulfo group.

In the present invention, the chromatic pigment may be used in combination of two or more kinds thereof. For example, green color may be formed in a combination of C. I. Pigment Green 7, C. I. Pigment Green 36, C. I. Pigment Yellow 139, and C. I. Pigment Yellow 185, or in a combination of C. I. Pigment Green 58, C. I. Pigment Yellow 150, and C. I. Pigment Yellow 185.

In addition, in a case where the chromatic pigment is used in combination of two or more kinds thereof, the combination of two or more chromatic pigments may form black. Examples of such a combination include the following aspects (1) to (7). In a case where two or more chromatic pigments are included in the coloring composition and the combination of two or more chromatic pigments forms black, the coloring composition according to the embodiment of the present invention can be preferably used as the near-infrared transmitting filter.

(1) aspect in which a red pigment and a blue pigment are contained.

(2) aspect in which a red pigment, a blue pigment, and a yellow pigment are contained.

(3) aspect in which a red pigment, a blue pigment, a yellow pigment, and a violet pigment are contained.

(4) aspect in which a red pigment, a blue pigment, a yellow pigment, a violet pigment, and a green pigment are contained.

(5) aspect in which a red pigment, a blue pigment, a yellow pigment, and a green pigment are contained.

(6) aspect in which a red pigment, a blue pigment, and a green pigment are contained.

(7) aspect in which a yellow pigment and a violet pigment are contained.

(White Pigment)

Examples of the white pigment include titanium oxide, strontium titanate, barium titanate, zinc oxide, magnesium oxide, zirconium oxide, aluminum oxide, barium sulfate, silica, talc, mica, aluminum hydroxide, calcium silicate, aluminum silicate, hollow resin particles, and zinc sulfide. The white pigment is preferably particles having a titanium atom, more preferably titanium oxide. In addition, the white pigment is preferably a particle having a refractive index of 2.10 or more with respect to light having a wavelength of 589 nm. The above-mentioned refractive index is preferably 2.10 to 3.00 and more preferably 2.50 to 2.75.

In addition, as the white pigment, the titanium oxide described in “Titanium Oxide-Physical Properties and Applied Technology, written by Manabu Kiyono, pages 13 to 45, published in Jun. 25, 1991, published by Gihodo Shuppan Co., Ltd.” can also be used.

The white pigment is not limited to a compound formed of a single inorganic substance, and may be particles combined with other materials. For example, it is preferable to use a particle having a pore or other materials therein, a particle having a large number of inorganic particles attached to a core particle, or a core-shell composite particle composed of a core particle formed of polymer particles and a shell layer formed of inorganic fine nanoparticles. With regard to the core-shell composite particle composed of a core particle formed of polymer particles and a shell layer formed of inorganic fine nanoparticles, reference can be made to, for example, the descriptions in paragraph Nos. 0012 to 0042 of JP2015-047520A, the contents of which are incorporated herein by reference.

As the white pigment, hollow inorganic particles can also be used. The hollow inorganic particles refer to inorganic particles having a structure with a cavity therein, and the cavity is enclosed by an outer shell. As the hollow inorganic particles, hollow inorganic particles described in JP2011-075786A, WO2013/061621A, JP2015-164881A, and the like can be used, the contents of which are incorporated herein by reference.

(Black Pigment)

The black pigment is not particularly limited, and a known black pigment can be used. Examples thereof include carbon black, titanium black, and graphite, and carbon black or titanium black is preferable and titanium black is more preferable. The titanium black is black particles containing a titanium atom, and is preferably lower titanium oxide or titanium oxynitride. The surface of the titanium black can be modified, as necessary, according to the purpose of improving dispersibility, suppressing aggregating properties, and the like. For example, the surface of the titanium black can be coated with silicon oxide, titanium oxide, germanium oxide, aluminum oxide, magnesium oxide, or zirconium oxide. In addition, a treatment with a water-repellent substance as described in JP2007-302836A can be performed. Examples of the black pigment include Color Index (C. I.) Pigment Black 1 and 7. It is preferable that the titanium black has a small primary particle diameter of the individual particles and has a small average primary particle diameter. Specifically, the average primary particle diameter thereof is preferably 10 to 45 nm. The titanium black can be used as a dispersion. Examples thereof include a dispersion which includes titanium black particles and silica particles and in which the content ratio of Si atoms to Ti atoms is adjusted to a range of 0.20 to 0.50. With regard to the dispersion, reference can be made to the description in paragraphs 0020 to 0105 of JP2012-169556A, the contents of which are incorporated herein by reference. Examples of a commercially available product of the titanium black include Titanium black 10S, 12S, 13R, 13M, 13M-C, 13R-N, 13M-T (trade name; manufactured by Mitsubishi Materials Corporation) and Tilack D (trade name; manufactured by Akokasei Co., Ltd.).

(Near-Infrared Absorbing Pigment)

The near-infrared absorbing pigment is preferably an organic pigment. In addition, the near-infrared absorbing pigment preferably has a maximal absorption wavelength in a wavelength range of more than 700 nm and 1400 nm or less. In addition, the maximal absorption wavelength of the near-infrared absorbing pigment is preferably 1200 nm or less, more preferably 1000 nm or less, and still more preferably 950 nm or less. In addition, in the near-infrared absorbing pigment, A₅₅₀/Aₘₐₓ, which is a ratio of an absorbance A₅₅₀ at a wavelength of 550 nm to an absorbance Aₘₐₓ at the maximal absorption wavelength, is preferably 0.1 or less, more preferably 0.05 or less, still more preferably 0.03 or less, and particularly preferably 0.02 or less. The lower limit is not particularly limited, but for example, may be 0.0001 or more or may be 0.0005 or more. In a case where the ratio of the above-described absorbance is within the above-described range, a near-infrared absorbing pigment excellent in visible transparency and near-infrared shielding property can be obtained. In the present invention, the maximal absorption wavelength of the near-infrared absorbing pigment and values of absorbance at each wavelength are values obtained from an absorption spectrum of a film formed by using a coloring composition including the near-infrared absorbing pigment.

The near-infrared absorbing pigment is not particularly limited, and examples thereof include a pyrrolopyrrole compound, a rylene compound, an oxonol compound, a squarylium compound, a cyanine compound, a croconium compound, a phthalocyanine compound, a naphthalocyanine compound, a pyrylium compound, an azurenium compound, an indigo compound, and a pyrromethene compound. Among these, at least one compound selected from a pyrrolopyrrole compound, a squarylium compound, a cyanine compound, a phthalocyanine compound, or a naphthalocyanine compound is preferable, and a pyrrolopyrrole compound or a squarylium compound is more preferable, and a pyrrolopyrrole compound is particularly preferable.

The content of the pigment in the total solid content of the coloring composition is 50% by mass or more, preferably 53% by mass or more, more preferably 55% by mass or more, and still more preferably 60% by mass or more. The upper limit is preferably 80% by mass or less, more preferably 75% by mass or less, and still more preferably 70% by mass or less.

The content of the green pigment in the total solid content of the coloring composition is preferably 30% by mass or more, more preferably 40% by mass or more, and still more preferably 45% by mass or more. The upper limit is preferably 70% by mass or less, more preferably 65% by mass or less, and still more preferably 60% by mass or less.

The content of the phthalocyanine pigment in the total solid content of the coloring composition is preferably 30% by mass or more, more preferably 30% by mass or more, and still more preferably 45% by mass or more. The upper limit is preferably 70% by mass or less, more preferably 65% by mass or less, and still more preferably 60% by mass or less.

<<Dye>>

The coloring composition according to the embodiment of the present invention can contain a dye. The dye is not particularly limited and a known dye can be used. The dye may be a chromatic dye or may be a near-infrared absorbing dye. Examples of the chromatic dye include a pyrazoleazo compound, an anilinoazo compound, a triarylmethane compound, an anthraquinone compound, an anthrapyridone compound, a benzylidene compound, an oxonol compound, a pyrazolotriazoleazo compound, a pyridoneazo compound, a cyanine compound, a phenothiazine compound, a pyrrolopyrazoleazomethine compound, a xanthene compound, a phthalocyanine compound, a benzopyran compound, an indigo compound, and a pyrromethene compound. In addition, the thiazole compound described in JP2012-158649A, the azo compound described in JP2011-184493A, or the azo compound described in JP2011-145540A can also be used. Examples of the near-infrared absorbing dye include a pyrrolopyrrole compound, a rylene compound, an oxonol compound, a squarylium compound, a cyanine compound, a croconium compound, a phthalocyanine compound, a naphthalocyanine compound, a pyrylium compound, an azurenium compound, an indigo compound, and a pyrromethene compound.

As the dye, a coloring agent multimer can also be used. The coloring agent multimer has two or more coloring agent structures in one molecule, and preferably has three or more coloring agent structures in one molecule. The upper limit is particularly not limited, but may be 100 or less. A plurality of coloring agent structures included in one molecule may be the same coloring agent structure or different coloring agent structures. The weight-average molecular weight (Mw) of the coloring agent multimer is preferably 2000 to 50000. The lower limit is more preferably 3000 or more and still more preferably 6000 or more. The upper limit is more preferably 30000 or less and still more preferably 20000 or less. As the coloring agent multimer, the compounds described in JP2011-213925A, JP2013-041097A, JP2015-028144A, JP2015-030742A, JP2016-102191A, WO2016/031442A, or the like can also be used.

The content of the dye in the total solid content of the coloring composition is preferably 1% by mass or more, more preferably 5% by mass or more, and particularly preferably 10% by mass or more. The upper limit is not particularly limited, but is preferably 70% by mass or less, more preferably 65% by mass or less, and still more preferably 60% by mass or less.

In addition, the content of the dye is preferably 5 to 50 parts by mass with respect to 100 parts by mass of the pigment. The upper limit is preferably 45 parts by mass or less and more preferably 40 parts by mass or less. The lower limit is preferably 10 parts by mass or more and still more preferably 15 parts by mass or more.

In addition, it is also possible that the coloring composition according to the embodiment of the present invention does not substantially contain the dye. The case where the coloring composition according to the embodiment of the present invention does not substantially include the dye means that the content of the dye in the total solid content of the coloring composition according to the embodiment of the present invention is preferably 0.1% by mass or less, more preferably 0.05% by mass or less, and particularly preferably 0% by mass.

<<Triazine Compound (TA)>>

The coloring composition according to the embodiment of the present invention includes a compound (triazine compound (TA)) that is a compound having a triazine ring, which includes at least one group selected from an acid group or a basic group, and in which a maximum value of a molar absorption coefficient of the compound in a wavelength range of 400 to 700 nm is 3000 L·mol⁻¹·cm⁻¹ or less.

The triazine compound (TA) preferably includes 2 or more triazine rings in one molecule, more preferably includes 2 to 4 triazine rings, and still more preferably 2 or 3 triazine rings. By including 2 or more triazine rings in one molecule, it is presumed that the network between the pigment and the triazine compound (TA) in the film is more likely to be formed more firmly, and it is easy to form a film having excellent adhesiveness with a support.

The acid group included in the triazine compound (TA) is preferably at least one selected from a carboxyl group, a sulfo group, a phosphoric acid group, or salts thereof, and more preferably at least one selected from a carboxyl group, a sulfo group, or salts thereof. Examples of an atom or atomic group constituting the salts include alkali metal ions (Li⁺, Na⁺, K⁺, and the like), alkaline earth metal ions (Ca²⁺, Mg²⁺, and the like), an ammonium ion, an imidazolium ion, a pyridinium ion, and a phosphonium ion.

The basic group included in the triazine compound (TA) is preferably at least one selected from an amino group, a pyridyl group, salts thereof, a salt of an ammonium group, or a phthalimidomethyl group, more preferably at least one selected from an amino group, a salt of an amino group, or a salt of an ammonium group, and more preferably an amino group or a salt of an amino group. Examples of the amino group include −NH₂, a dialkylamino group, an alkylarylamino group, a diarylamino group, and a cyclic amino group. The dialkylamino group, alkylarylamino group, diarylamino group, and cyclic amino group may further have a substituent. Examples of the substituent include the substituent T described later. Examples of an atom or atomic group constituting the salts include a hydroxide ion, a halogen ion, a carboxylate ion, a sulfonate ion, and a phenoxide ion.

From the reason that color unevenness can be more easily suppressed, the triazine compound (TA) is preferably a compound including the basic group.

The molecular weight of the triazine compound (TA) is preferably 400 to 1500, more preferably 450 to 1250, and still more preferably 500 to 1000. In a case where the molecular weight of the triazine compound (TA) is within the above-described range, storage stability of the coloring composition can be further improved.

From the viewpoint of stability of the triazine compound, the ethylenically unsaturated bond-containing group (hereinafter, referred to as a C═C value) of the triazine compound (TA) is preferably 0.0050 mmol/g or less, more preferably 0.0035 mmol/g or less, still more preferably 0.0030 mmol/g or less, even more preferably 0.0025 mmol/g or less, and particularly preferably 0 mmol/g. The C═C value of the triazine compound (TA) is a value calculated by dividing the number of ethylenically unsaturated bond-containing groups included in one molecule of the triazine compound (TA) by the molecular weight of the triazine compound (TA).

The triazine compound (TA) is preferably a compound including a group represented by Formula (C1). In a case where the triazine compound (TA) is a compound including such a group, the effects of the present invention are more remarkably obtained.

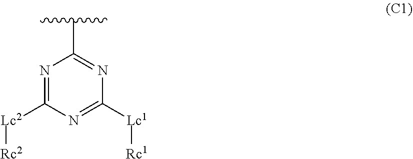

In the formula, a wavy line represents a bonding site, Lc¹ and Lc² each independently represent a single bond or a linking group, Rc¹ and Rc² each independently represent a substituent, and at least one of Rc¹ or Rc² represents an acid group or a basic group.

In Formula (C1), Lc¹ and Lc² each independently represent a single bond or a linking group, and a divalent linking group is preferable. Examples of the divalent linking group include an alkylene group, an arylene group, —O—, —NHCO—, —CONH—, —OCO—, —COO——CO—, —SO₂NH—, —SO₂—, and a group formed by a combination of these groups. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{L1} represents a hydrogen atom, an alkyl group, or an aryl group, and a hydrogen atom or an alkyl group is preferable and a hydrogen atom is more preferable. The alkyl group represented by R^{L1} preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The alkyl group represented by R^{L1} may further have a substituent. Examples of the substituent include the substituent T described later. The aryl group represented by R^{L1} preferably has 6 to 30 carbon atoms, more preferably has 6 to 20 carbon atoms, and still more preferably has 6 to 12 carbon atoms. The aryl group represented by R^{L1} may further have a substituent. Examples of the substituent include the substituent T described later.

In Formula (C1), Rc¹ and Rc² each independently represent a substituent. Examples of the substituent include an alkyl group, an aryl group, a heterocyclic group, a hydroxy group, an acid group, and a basic group. However, at least one of Rc¹ or Rc² represents an acid group or a basic group. It is preferable that at least one of Rc¹ or Rc² is a basic group, and it is more preferable that both Rc¹ and Rc² are basic groups. Examples of the acid group and the basic group include those described above. The alkyl group preferably has 1 to 30 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The heterocyclic group may be monocyclic or a fused ring. The heterocyclic group is preferably monocyclic or a fused ring having 2 to 4 fused rings. The number of heteroatoms constituting a ring of the heterocyclic group is preferably 1 to 3. The heteroatom constituting the ring of the heterocyclic group is preferably a nitrogen atom, an oxygen atom, or a sulfur atom. The number of carbon atoms constituting the ring of the heterocyclic group is preferably 3 to 30, more preferably 3 to 18, and more preferably 3 to 12. The alkyl group, aryl group, and heterocyclic group may further have a substituent. Examples of the substituent include the substituent T shown below.

(Substituent T)

Examples of a substituent T include a halogen atom, a cyano group, a nitro group, an alkyl group, an alkenyl group, an alkynyl group, an aryl group, a heterocyclic group, -ORt¹, —CORt¹, —COORt¹, —OCORt¹, —NRt¹Rt², —NHCORt¹, —CONRt¹Rt², —NHCONRt¹Rt², —NHCOORt¹, —SRt¹, —SO₂Rt¹, —SO₂ORt¹, —NHSO₂Rt¹, and —SO₂NRt¹Rt². Rt¹ and Rt² each independently represent a hydrogen atom, an alkyl group, an alkenyl group, an alkynyl group, an aryl group, or a heteroaryl group. Rt¹ and Rt² may be bonded to each other to form a ring. In a case where Rt¹ in —COORt¹ is hydrogen atom, the hydrogen atom may be dissociated or in a form of salt. In addition, in a case where Rt¹ in —SO₂ORt¹ is a hydrogen atom, the hydrogen atom may be dissociated or in a form of salt.

Examples of the halogen atom include a fluorine atom, a chlorine atom, a bromine atom, and an iodine atom.

The alkyl group preferably has 1 to 30 carbon atoms, more preferably has 1 to 15 carbon atoms, and still more preferably has 1 to 8 carbon atoms. The alkyl group may be any of linear, branched, and cyclic forms, and is preferably linear or branched and more preferably linear.

The alkenyl group preferably has 2 to 30 carbon atoms, more preferably has 2 to 12 carbon atoms, and particularly preferably has 2 to 8 carbon atoms. The alkenyl group may be any of linear, branched, and cyclic forms, and is preferably linear or branched and more preferably linear.

The alkynyl group preferably has 2 to 30 carbon atoms and more preferably has 2 to 25 carbon atoms. The alkynyl group may be any of linear, branched, and cyclic forms, and is preferably linear or branched and more preferably linear.

The aryl group preferably has 6 to 30 carbon atoms, more preferably has 6 to 20 carbon atoms, and still more preferably has 6 to 12 carbon atoms.

The heterocyclic group may be monocyclic or a fused ring. The heterocyclic group is preferably monocyclic or a fused ring having 2 to 4 fused rings. The number of heteroatoms constituting a ring of the heterocyclic group is preferably 1 to 3. The heteroatom constituting the ring of the heterocyclic group is preferably a nitrogen atom, an oxygen atom, or a sulfur atom. The number of carbon atoms constituting the ring of the heterocyclic group is preferably 3 to 30, more preferably 3 to 18, and more preferably 3 to 12.

The alkyl group, the alkenyl group, the alkynyl group, the aryl group, and the heterocyclic group may have a substituent or may be unsubstituted. Examples of the substituent include the substituents described in the substituent T.

The group represented by Formula (C1) is preferably a group represented by Formula (C2), and more preferably a group represented by Formula (C3).

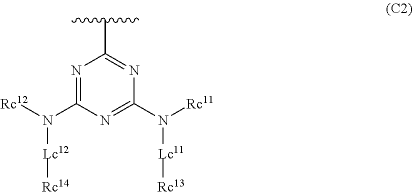

In Formula (C2), a wavy line represents a bonding site, Lc¹¹ and Lc¹² each independently represent a single bond or a linking group, Rc¹¹ and Rc¹² each independently represent a hydrogen atom or a substituent, Rc¹³ and Rc¹⁴ each independently represent a substituent, and at least one of Rc¹³ or Rc¹⁴ represents an acid group or a basic group.

Rc¹³ and Rc¹⁴ in Formula (C2) have the same meaning as Rc¹ and Rc² in Formula (C1), and the preferred ranges are also the same.

In Formula (C2), Rc¹¹ and Rc¹² each independently represent a hydrogen atom or a substituent. Examples of the substituent represented by Rc¹¹ and Rc¹² include an alkyl group and an aryl group. The alkyl group preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The alkyl group and aryl group may further have a substituent. Examples of the substituent include the above-described substituent T.

In Formula (C2), Rc¹¹ and Rc¹² are preferably hydrogen atoms.

In Formula (C2), Lc¹¹ and Lc¹² each independently represent a single bond or a linking group, and a divalent linking group is preferable. Examples of the divalent linking group include an alkylene group, an arylene group, —O—, —N(R^{L11})—, —NHCO—, —CONH—, —OCO—, —COO—, —CO—, —SO₂NH—, —SO₂—, and a group formed by a combination of these groups, and a group including at least one selected from an alkylene group or an arylene group is preferable, a group including an alkylene group is more preferable, and an alkylene group is still more preferable. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{L11} represents a hydrogen atom, an alkyl group, or an aryl group, and a hydrogen atom or an alkyl group is preferable and a hydrogen atom is more preferable. The alkyl group and aryl group represented by R^{L11} have the same meaning as the alkyl group and aryl group represented by R^{L1} described above.

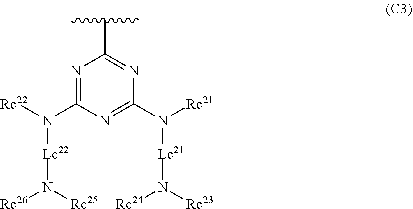

In Formula (C3), a wavy line represents a bonding site, Lc²¹ and Lc²² each independently represent a single bond or a linking group, Rc²¹ and Rc²² each independently represent a hydrogen atom or a substituent, Rc²³ to Rc²⁶ each independently represent a hydrogen atom or a substituent, Rc²³ and Rc²⁴ may be bonded to each other through a divalent group to form a ring, and Rc²⁵ and Rc²⁶ may be bonded to each other through a divalent group to form a ring.

Rc²¹ and Rc²² in Formula (C3) have the same meaning as Rc¹¹ and Rc¹² in Formula (C2), and the preferred ranges are also the same. Lc²¹ and Lc²² in Formula (C3) have the same meaning as Lc¹¹ and Lc¹² in Formula (C2), and the preferred ranges are also the same.

In Formula (C3), Rc²³ and Rc²⁶ each independently represent a hydrogen atom or a substituent, and a substituent is preferable. Examples of the substituent include an alkyl group and an aryl group, and an alkyl group is preferable. The alkyl group preferably has 1 to 10 carbon atoms, more preferably has 1 to 5 carbon atoms, and still more preferably has 1 to 3 carbon atoms. The alkyl group may be any of linear, branched, and cyclic forms, and is preferably linear or branched and more preferably linear. The aryl group preferably has 6 to 30 carbon atoms, more preferably has 6 to 20 carbon atoms, and still more preferably has 6 to 12 carbon atoms. The alkyl group and aryl group may further have a substituent. Examples of the substituent include the above-described substituent T.

In Formula (C3), Rc²³ and Rc²⁴ may be bonded to each other through a divalent group to form a ring, and Rc²⁵ and Rc²⁶ may be bonded to each other through a divalent group to form a ring. Examples of the divalent group include —CH₂—, —O—, and —SO₂—. Specific examples of the ring formed by bonding the above-described groups to each other through the divalent group include the following.

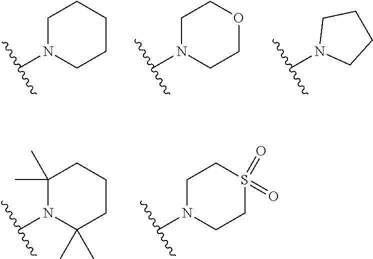 [SMILES: *N1CCCCC1 ; *N1CCOCC1 ; *N1CCCC1 ; *N1C(C)(C)CCCC1(C)C ; *N1CCS(=O)(=O)CC1]

Specific examples of the group represented by Formula (C1) include groups having the following structures.

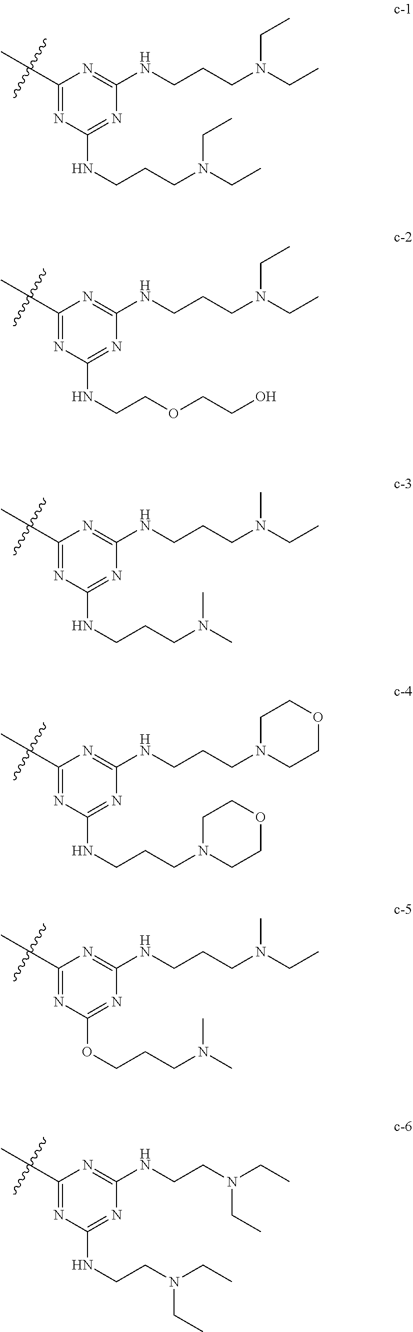 [SMILES: *c1nc(NCCCN(CC)CC)nc(NCCCN(CC)CC)n1 ; *c1nc(NCCCN(CC)CC)nc(NCCOCCO)n1 ; *c1nc(NCCCN(C)C)nc(NCCCN(C)CC)n1 ; *c1nc(NCCCN2CCOCC2)nc(NCCCN2CCOCC2)n1 ; *c1nc(NCCCN(C)CC)nc(OCCCN(C)C)n1 ; *c1nc(NCCN(CC)CC)nc(NCCN(CC)CC)n1]

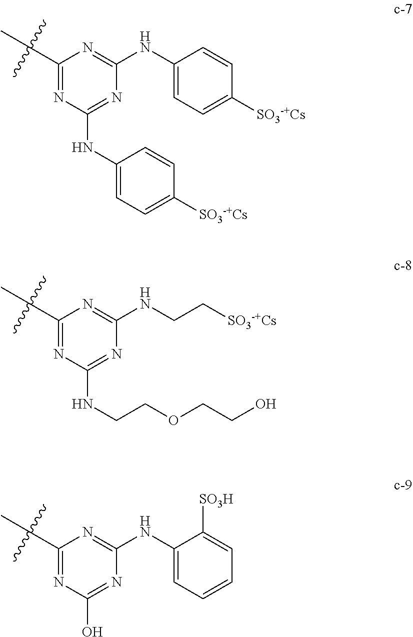

The triazine compound (TA) is preferably a compound represented by Formula (1).

A¹-B¹—C¹ (1)

In Formula (1), A¹ represents a group including an aromatic ring, B¹ represents a single bond or a divalent linking group, and C¹ represents the group represented by Formula (C1).

C¹ in Formula (1) represents the group represented by Formula (C1), and is preferably the group represented by Formula (C2) and more preferably the group represented by Formula (C3).

In Formula (1), A¹ represents a group including an aromatic ring. The aromatic ring may be an aromatic hydrocarbon ring or an aromatic heterocyclic ring. In addition, the aromatic ring may be monocyclic or a fused ring.

Examples of the group represented by A¹ include a group including an aromatic ring selected from a benzene ring, a naphthalene ring, a fluorene ring, a perylene ring, an imidazole ring, a pyrazole ring, an oxazole ring, a thiazole ring, an imidazoline ring, a pyridine ring, a triazole ring, an imidazoline ring, a pyrazine ring, a pyrimidine ring, a pyridazine ring, a quinoline ring, an isoquinoline ring, a quinoxaline ring, a quinazoline ring, a benzimidazole ring, a benzopyrazole ring, a benzoxazole ring, a benzothiazole ring, a benzotriazole ring, an indole ring, an isoindole ring, a triazine ring, a pyrrole ring, a carbazole ring, a benzimidazolinone ring, a phthalimide ring, a phthalocyanine ring, an anthraquinone ring, a diketopyrrolopyrrole ring, an isoindolinone ring, an isoindoline ring, and a quinacridone ring; and a group including a fused ring which includes these aromatic rings. The above-described fused ring may be an aromatic ring or a non-aromatic ring, but is preferably an aromatic ring.

In addition, the group represented by A¹ may be a group having only one aromatic ring or fused ring described above, but for the reason that, as the number of aromatic rings increases, pigment adsorbability is improved and storage stability of the composition is easily improved by π-π interaction, it is preferable to have two or more of these rings.

The group represented by A¹ may further have a substituent. Examples of the substituent include the above-described substituent T.

The group represented by A¹ is preferably a group having a structure which easily interacts with the pigment included in the coloring composition or a structure similar to the pigment. According to this aspect, dispersibility of the pigment in the coloring composition can be enhanced, and storage stability of the coloring composition can be further enhanced. In addition, from the reason that the effects of the present invention are more easily obtained, the group represented by A¹ is preferably a group including an aromatic heterocyclic ring, more preferably a group including a nitrogen-containing aromatic heterocyclic ring, still more preferably a group including a triazine ring, and particularly preferably a group represented by Formula (A1).

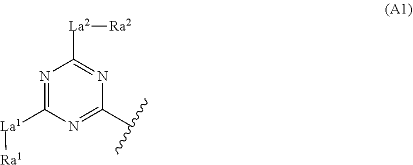

In the formula, a wavy line represents a bonding site, La¹ and La² each independently represent a single bond or a divalent linking group, and Ra¹ and Ra² each independently represent a hydrogen atom or a substituent.

In Formula (A1), La¹ and La² each independently represent a single bond or a linking group, and a divalent linking group is preferable. Examples of the divalent linking group include an alkylene group, an arylene group, —O—, —N(R^{La1})—, —NHCO—, —CONH—, —OCO—, —COO—, —CO—, —SO₂NH—, —SO₂—, and a group formed by a combination of these groups. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{La1} represents a hydrogen atom, an alkyl group, or an aryl group, and a hydrogen atom or an alkyl group is preferable and a hydrogen atom is more preferable. The alkyl group represented by R^{La1} preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The alkyl group represented by R^{La1} may further have a substituent. Examples of the substituent include the above-described substituent T. The aryl group represented by R^{La1} preferably has 6 to 30 carbon atoms, more preferably has 6 to 20 carbon atoms, and still more preferably has 6 to 12 carbon atoms. The aryl group represented by R^{La1} may further have a substituent. Examples of the substituent include the above-described substituent T.

The divalent linking group represented by La¹ and La² is preferably —N(R^{La1})— or —O—, and more preferably —N(R^{La1})—.

In Formula (A1), Ra¹ and Ra² each independently represent a hydrogen atom or a substituent. Examples of the substituent include the above-described substituent T, and an alkyl group, an aryl group, or a heterocyclic group is preferable, an aryl group or a heterocyclic group is more preferable, and an aryl group is still more preferable from the reason that pigment adsorbability is enhanced and storage stability of the composition is easily improved. The alkyl group, aryl group, and heterocyclic group represented by Ra¹ and Ra² may further have a substituent. Examples of the further substituent include the above-described substituent T.

In addition, from the reason that color unevenness can be more easily suppressed, it is preferable that at least one of Ra¹ or Ra² is a group including a structure selected from a urea structure, an imide structure, and an amide structure, and it is more preferable to be a group including a urea structure, and it is still more preferable to be a heterocyclic group including a urea structure. Examples of the heterocyclic group including a urea structure include a benzimidazolone group.

Specific examples of A¹ include groups having the following structures. In the following structural formulae, Me represents a methyl group.

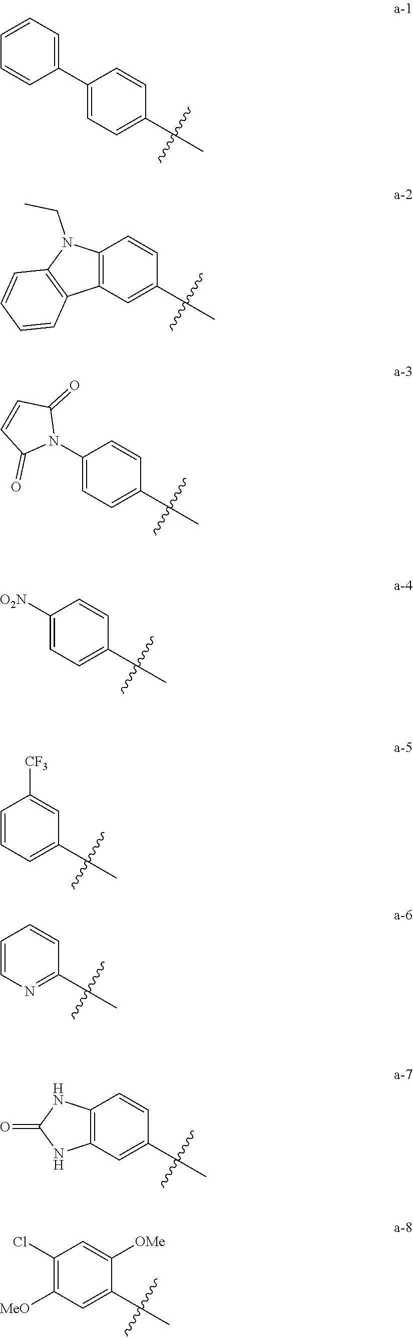

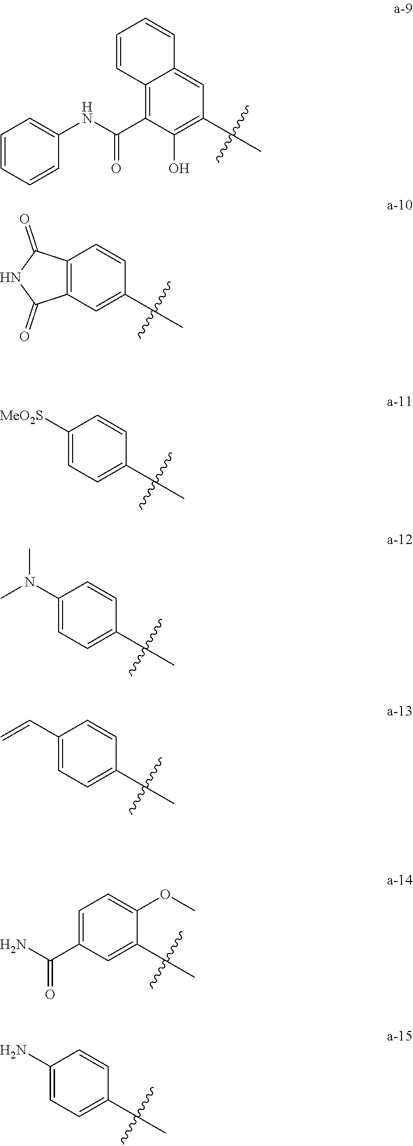

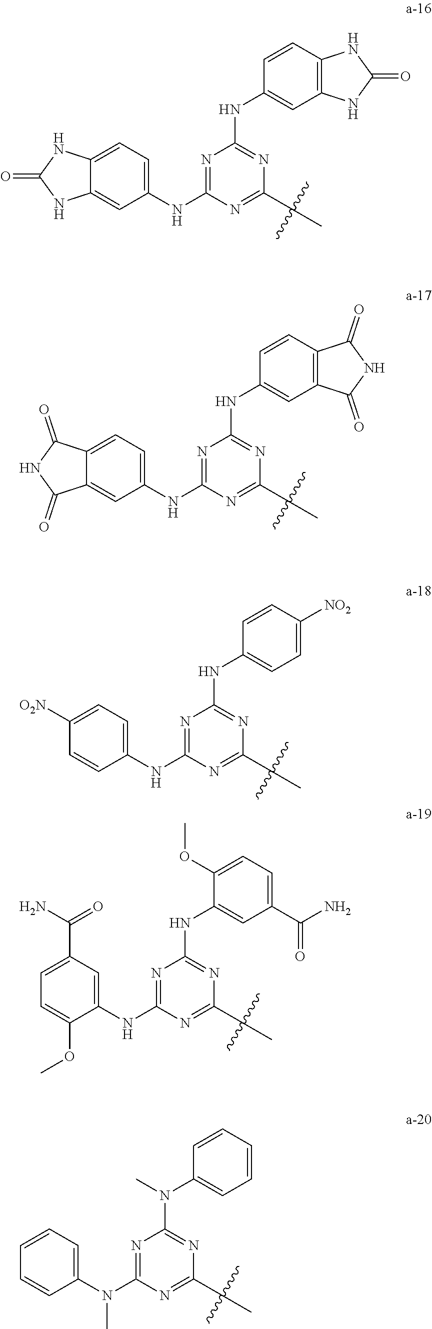

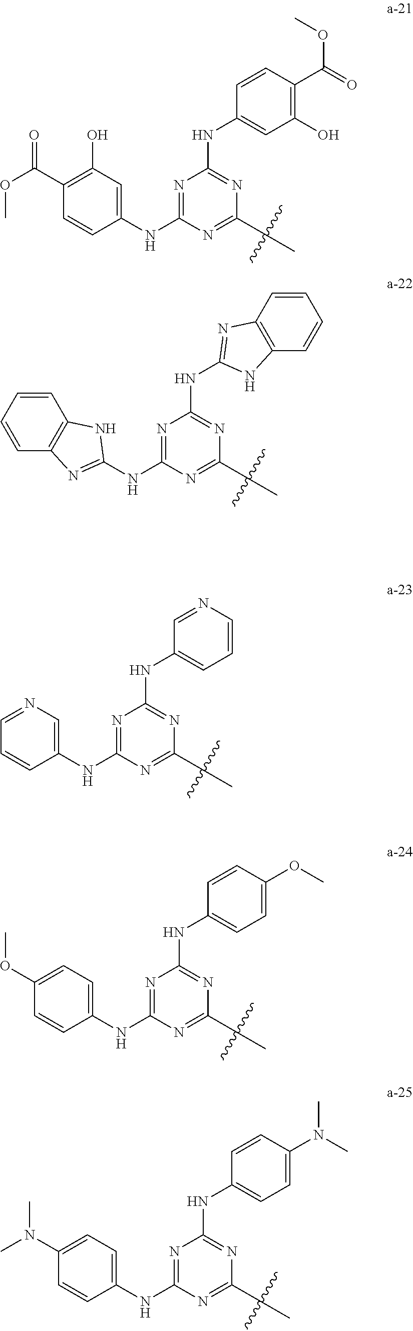

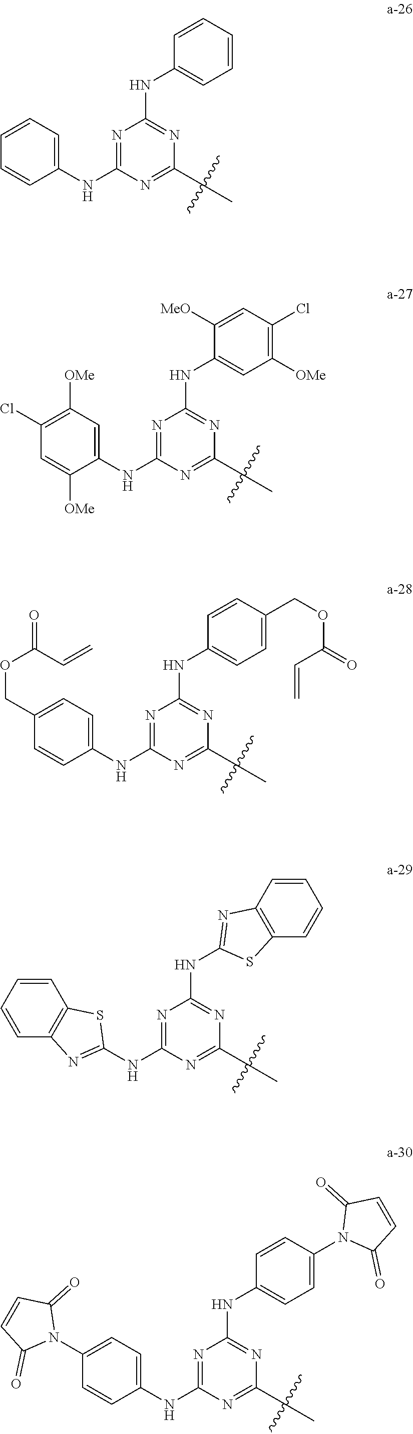

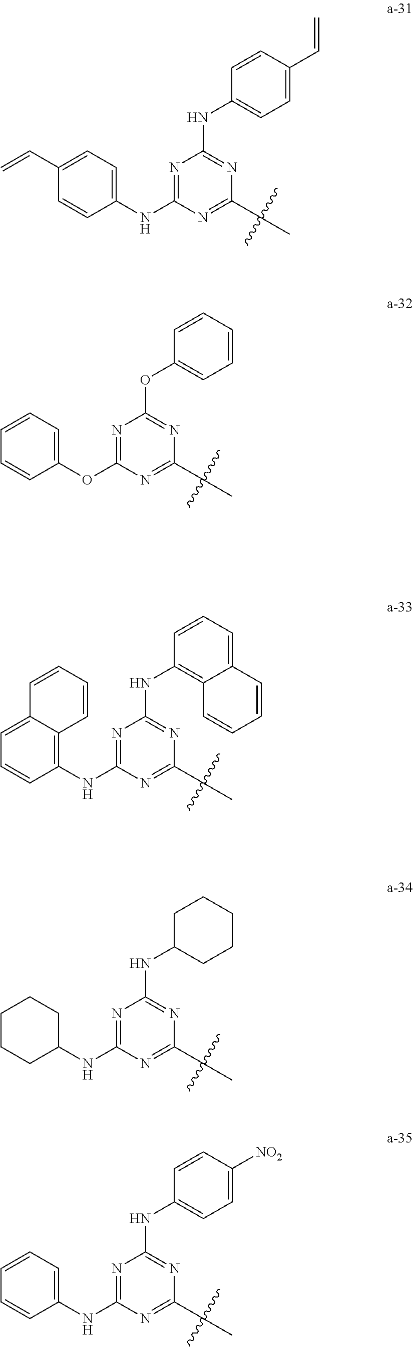

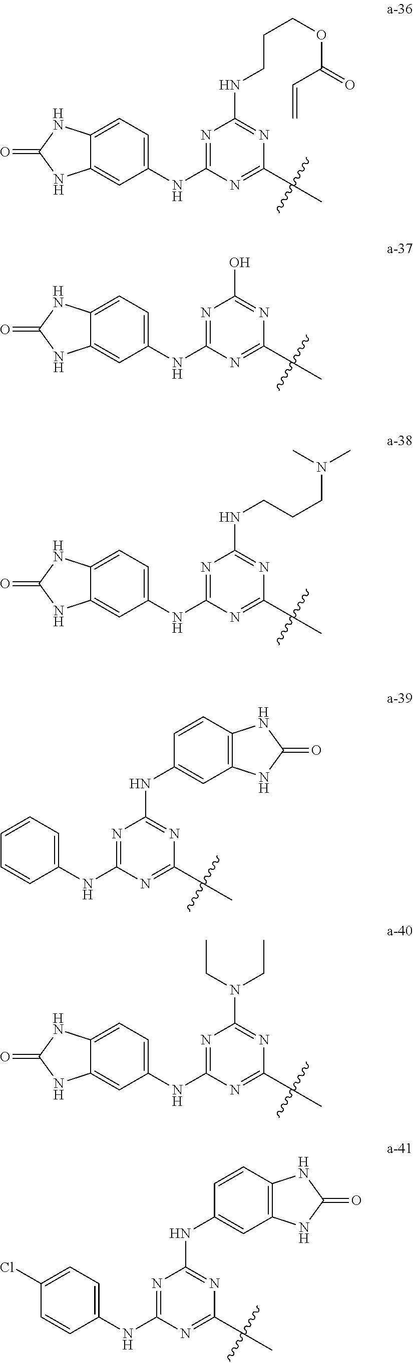

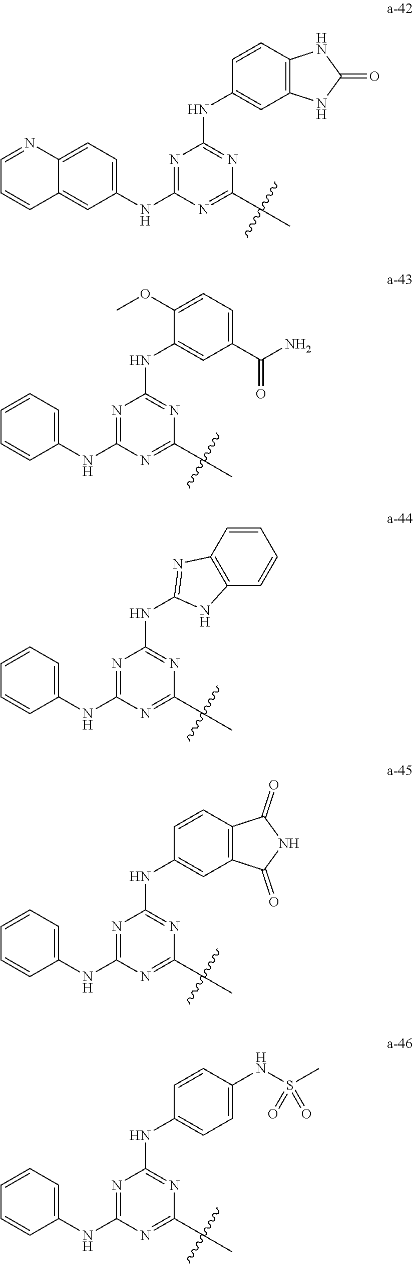

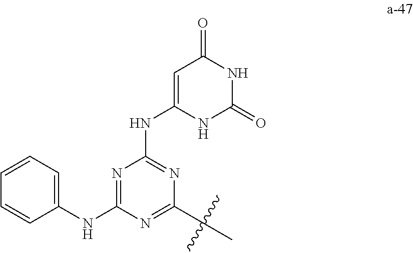

In Formula (1), B¹ represents a single bond or a divalent linking group, and a divalent linking group is preferable. Examples of the divalent linking group represented by B¹ include an alkylene group, an arylene group, —O—, —N(R^{LB1})—, —NHCO—, —CONH—, —OCO—, —COO—, —CO—, —SO₂NH—, —SO₂—, and a group formed by a combination of these groups. The alkylene group preferably has 1 to 30 carbon atoms, more preferably has 1 to 15 carbon atoms, still more preferably has 1 to 8 carbon atoms, and particularly preferably has 1 to 5 carbon atoms. The alkylene group may be any of linear, branched, and cyclic forms, and is preferably linear or branched and particularly preferably linear. The arylene group preferably has 6 to 30 carbon atoms and more preferably has 6 to 15 carbon atoms. The arylene group is preferably a phenylene group. R^{LB1} represents a hydrogen atom, an alkyl group, or an aryl group, and a hydrogen atom or an alkyl group is preferable and a hydrogen atom is more preferable. The alkyl group represented by R^{LB1} preferably has 1 to 20 carbon atoms, more preferably has 1 to 15 carbon atoms, and still more preferably has 1 to 8 carbon atoms. The alkyl group may be any of linear, branched, and cyclic forms, and is preferably linear or branched and more preferably linear. The alkyl group represented by R^{LB1} may further have a substituent. Examples of the substituent include the above-described substituent T. The aryl group represented by R^{LB1} preferably has 6 to 30 carbon atoms, more preferably has 6 to 20 carbon atoms, and still more preferably has 6 to 12 carbon atoms. The aryl group represented by R^{LB1} may further have a substituent. Examples of the substituent include the above-described substituent T.

The divalent linking group represented by B¹ is preferably a group represented by Formula (L1).

—L^{1A}-L^{1B}-L^{1C}— (L1)

In the formula, L^{1A} and L^{1C} each independently represent —O—, —N(R^{LB1})—, —NHCO—, —CONH—, —OCO—, —COO——CO—, —SO₂NH—, or —SO₂—, and L^{1B} represents a single bond or a divalent linking group.

Examples of the divalent linking group represented by L^{1B} include an alkylene group, an arylene group, a group in which an alkylene group and an arylene group are bonded to each other through a single bond, —O—, —N(R^{LB1})—, —NHCO—, —CONH—, —OCO—, —COO——CO—, —SO₂NH—, —SO₂—, or a group formed by a combination of these groups, and a group in which alkylene groups or arylene groups are bonded to each other through —O—, —N(R^{LB1})—, —NHCO—, —CONH—, —OCO—, —COO——CO—, —SO₂NH—, —SO₂—, or a group formed by a combination of these groups.

Specific examples of B¹ include groups having the following structures.

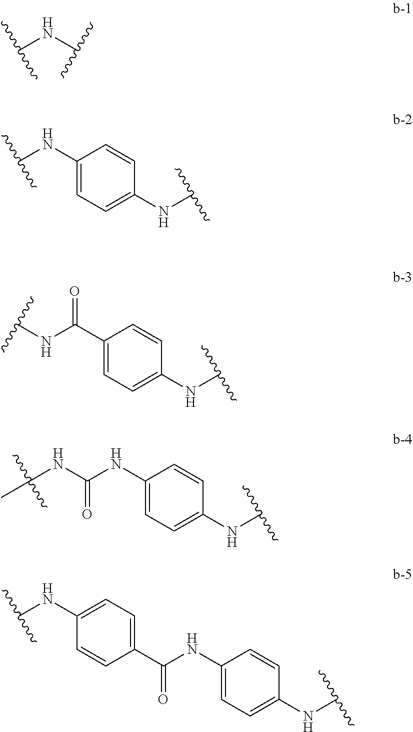

Specific examples of the triazine compound (TA) include the following. In the following table, the symbols described in the columns of structure of A¹, structure of B¹, and structure of C¹ are the structures exemplified in the specific examples of A¹, the specific examples of B¹, and the specific examples of C¹ respectively.

TABLE 1
A¹-B¹-C¹

| Com- | | | |
|---|---|---|---|
| pound | Structure | Structure | Structure |
| No | of A¹ | of B¹ | of C¹ |
| T-1 | a-1 | b-1 | c-1 |
| T-2 | a-2 | b-1 | c-1 |
| T-3 | a-3 | b-1 | c-1 |
| T-4 | a-4 | b-1 | c-1 |
| T-5 | a-5 | b-1 | c-1 |
| T-6 | a-6 | b-1 | c-1 |
| T-7 | a-7 | b-1 | c-1 |
| T-8 | a-8 | b-1 | c-1 |
| T-9 | a-9 | b-1 | c-1 |
| T-10 | a-10 | b-1 | c-1 |
| T-11 | a-11 | b-1 | c-1 |
| T-12 | a-12 | b-1 | c-1 |
| T-13 | a-13 | b-1 | c-1 |
| T-14 | a-14 | b-1 | c-1 |
| T-15 | a-15 | b-1 | c-1 |
| T-16 | a-7 | b-3 | c-1 |
| T-17 | a-9 | b-4 | c-1 |
| T-18 | a-10 | b-3 | c-1 |
| T-19 | a-14 | b-4 | c-1 |
| T-20 | a-7 | b-1 | c-3 |
| T-21 | a-7 | b-1 | c-4 |
| T-22 | a-7 | b-1 | c-7 |
| T-23 | a-16 | b-2 | c-1 |
| T-24 | a-17 | b-2 | c-1 |
| T-25 | a-18 | b-2 | c-1 |
| T-26 | a-19 | b-2 | c-1 |
| T-27 | a-20 | b-2 | c-1 |
| T-28 | a-21 | b-2 | c-1 |
| T-29 | a-22 | b-2 | c-1 |
| T-30 | a-23 | b-2 | c-1 |
| T-31 | a-24 | b-2 | c-1 |
| T-32 | a-25 | b-2 | c-1 |
| T-33 | a-26 | b-2 | c-1 |
| T-34 | a-27 | b-2 | c-1 |
| T-35 | a-28 | b-2 | c-1 |
| T-36 | a-29 | b-2 | c-1 |
| T-37 | a-30 | b-2 | c-1 |
| T-38 | a-31 | b-2 | c-1 |
| T-39 | a-32 | b-2 | c-1 |
| T-40 | a-33 | b-2 | c-1 |
| T-41 | a-34 | b-2 | c-1 |
| T-42 | a-35 | b-2 | c-1 |
| T-43 | a-36 | b-2 | c-1 |
| T-44 | a-37 | b-2 | c-1 |
| T-45 | a-38 | b-2 | c-1 |
| T-46 | a-39 | b-2 | c-1 |
| T-47 | a-40 | b-2 | c-1 |
| T-48 | a-41 | b-2 | c-1 |
| T-49 | a-42 | b-2 | c-1 |
| T-50 | a-43 | b-2 | c-1 |
| T-51 | a-44 | b-2 | c-1 |
| T-52 | a-45 | b-2 | c-1 |
| T-53 | a-46 | b-2 | c-1 |
| T-54 | a-47 | b-2 | c-1 |
| T-55 | a-16 | b-5 | c-1 |
| T-56 | a-17 | b-5 | c-1 |
| T-57 | a-19 | b-5 | c-1 |
| T-58 | a-26 | b-5 | c-1 |
| T-59 | a-36 | b-5 | c-1 |
| T-60 | a-37 | b-5 | c-1 |
| T-61 | a-38 | b-5 | c-1 |
| T-62 | a-39 | b-5 | c-1 |
| T-63 | a-40 | b-5 | c-1 |
| T-64 | a-41 | b-5 | c-1 |
| T-65 | a-42 | b-5 | c-1 |
| T-66 | a-43 | b-5 | c-1 |
| T-67 | a-44 | b-5 | c-1 |
| T-68 | a-45 | b-5 | c-1 |
| T-69 | a-46 | b-5 | c-1 |
| T-70 | a-47 | b-5 | c-1 |
| T-71 | a-39 | b-2 | c-2 |
| T-72 | a-41 | b-2 | c-3 |
| T-73 | a-43 | b-2 | c-4 |
| T-74 | a-39 | b-2 | c-5 |
| T-75 | a-39 | b-2 | c-6 |
| T-76 | a-39 | b-2 | c-7 |
| T-77 | a-39 | b-2 | c-8 |
| T-78 | a-39 | b-2 | c-9 |

In the triazine compound (TA), the maximum value of a molar absorption coefficient in a wavelength range of 400 to 700 nm is preferably 1000 L·mol⁻¹·cm⁻¹ or less and more preferably 100 L·mol⁻¹·cm⁻¹ or less. According to this aspect, adhesiveness of a film to be obtained with a support is easily further improved. In the present specification, the value of the molar absorption coefficient of the triazine compound (TA) is a value measured by a method described in Examples described later.

It is also preferable that the triazine compound (TA) satisfies any one of the following spectral characteristics (a) to (d).

(a) maximum value of the molar absorption coefficient in a wavelength range of more than 700 nm and 750 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

(b) maximum value of the molar absorption coefficient in a wavelength range of more than 750 nm and 800 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

(c) maximum value of the molar absorption coefficient in a wavelength range of more than 800 nm and 850 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

(d) maximum value of the molar absorption coefficient in a wavelength range of more than 850 nm and 900 nm or less is preferably 3000 L·mol⁻¹·cm⁻¹ or less, more preferably 1000 L·mol⁻¹·cm⁻¹ or less, and still more preferably 100 L·mol⁻¹·cm⁻¹ or less.

The content of the triazine compound (TA) in the total solid content of the coloring composition is preferably 0.3% to 20% by mass. The lower limit is preferably 0.6% by mass or more and more preferably 0.9% by mass or more. The upper limit is preferably 15% by mass or less, more preferably 12.5% by mass or less, and still more preferably 10% by mass or less.

In addition, the content of the triazine compound (TA) is preferably 1 to 30 parts by mass with respect to 100 parts by mass of the pigment. The lower limit is preferably 2% by mass or more and more preferably 3% by mass or more. The upper limit is preferably 20 parts by mass or less and more preferably 15% by mass or less. The compound (1) may be used singly or in combination of two or more kinds thereof. In a case where two or more kinds thereof are used in combination, the total amount thereof is preferably within the above-described range.

<<Polymerizable Compound>>

The coloring composition according to the embodiment of the present invention contains a polymerizable compound. As the polymerizable compound, a known compound which is cross-linkable by a radical, an acid, or heat can be used. The polymerizable compound is preferably, for example, a compound having an ethylenically unsaturated bond-containing group. Examples of the ethylenically unsaturated bond-containing group include a vinyl group, a (meth)allyl group, and a (meth)acryloyl group. The polymerizable compound used in the present invention is preferably a radically polymerizable compound.

Any chemical forms of a monomer, a prepolymer, an oligomer, or the like may be used as the polymerizable compound, but a monomer is preferable. The molecular weight of the polymerizable compound is preferably 100 to 3000. The upper limit is preferably 2000 or less and more preferably 1500 or less. The lower limit is preferably 150 or more and more preferably 250 or more.

The polymerizable compound is preferably a compound including 3 or more ethylenically unsaturated bond-containing groups, more preferably a compound including 3 to 15 ethylenically unsaturated bond-containing groups, and still more preferably a compound having 3 to 6 ethylenically unsaturated bond-containing groups. In addition, the polymerizable compound is preferably a trifunctional to pentadecafunctional (meth)acrylate compound and more preferably a trifunctional to hexafunctional (meth)acrylate compound. Specific examples of the polymerizable compound include the compounds described in paragraph Nos. 0095 to 0108 of JP2009-288705A, paragraph No. 0227 of JP2013-029760A, paragraph Nos. 0254 to 0257 of JP2008-292970A, paragraph Nos. 0034 to 0038 of JP2013-253224A, paragraph No. 0477 of JP2012-208494A, JP2017-048367A, JP6057891B, JP6031807B, and JP2017-194662A, the contents of which are incorporated herein by reference.

As the polymerizable compound, dipentaerythritol triacrylate (as a commercially available product, KAYARAD D-330 manufactured by Nippon Kayaku Co., Ltd.), dipentaerythritol tetraacrylate (as a commercially available product, KAYARAD D-320 manufactured by Nippon Kayaku Co., Ltd.), dipentaerythritol penta(meth)acrylate (as a commercially available product, KAYARAD D-310 manufactured by Nippon Kayaku Co., Ltd.), dipentaerythritol hexa(meth)acrylate (as a commercially available product, KAYARAD DPHA manufactured by Nippon Kayaku Co., Ltd., NK ESTER A-DPH-12E manufactured by Shin-Nakamura Chemical Co., Ltd.), or a compound having a structure in which these (meth)acryloyl groups are bonded through an ethylene glycol and/or a propylene glycol residue (for example, SR454 and SR499 which are commercially available products from Sartomer) is preferable. In addition, as the polymerizable compound, diglycerin ethylene oxide (EO)-modified (meth)acrylate (as a commercially available product, M-460 manufactured by TOAGOSEI CO., LTD.), pentaerythritol tetraacrylate (NK ESTER A-TMMT manufactured by Shin-Nakamura Chemical Co., Ltd.), 1,6-hexanediol diacrylate (KAYARAD HDDA manufactured by Nippon Kayaku Co., Ltd.), RP-1040 (manufactured by Nippon Kayaku Co., Ltd.), ARONIX TO-2349 (manufactured by TOAGOSEI CO., LTD.), NK OLIGO UA-7200 (manufactured by Shin-Nakamura Chemical Co., Ltd.), 8UH-1006 and 8UH-1012 (manufactured by Taisei Fine Chemical Co., Ltd.), Light Acrylate POB-A0 (manufactured by KYOEISHA CHEMICAL Co., Ltd.), and the like can also be used.

In addition, as the polymerizable compound, it is also preferable to use a trifunctional (meth)acrylate compound such as trimethylolpropane tri(meth)acrylate, trimethylolpropane propyleneoxide-modified tri(meth)acrylate, trimethylolpropane ethyleneoxide-modified tri(meth)acrylate, isocyanuric acid ethyleneoxide-modified tri(meth)acrylate, and pentaerythritol tri(meth)acrylate. Examples of a commercially available product of the trifunctional (meth)acrylate compound include ARONIX M-309, M-310, M-321, M-350, M-360, M-313, M-315, M-306, M-305, M-303, M-452, and M-450 (manufactured by TOAGOSEI CO., LTD.), NK ESTER A9300, A-GLY-9E, A-GLY-20E, A-TMM-3, A-TMM-3L, A -TMM-3LM-N, A-TMPT, and TMPT (manufactured by Shin-Nakamura Chemical Co., Ltd.), and KAYARAD GPO-303, TMPTA, THE-330, TPA-330, and PET-30 (manufactured by Nippon Kayaku Co., Ltd.).

As the polymerizable compound, a compound having an acid group can also be used. By using a polymerizable compound having an acid group, the coloring composition in a non-exposed portion is easily removed during development and the generation of development residue can be suppressed. Examples of the acid group include a carboxyl group, a sulfo group, and a phosphoric acid group, and a carboxyl group is preferable. Examples of a commercially available product of the polymerizable compound having an acid group include ARONIX M-305, M-510, M-520, and ARONIX TO-2349 (manufactured by TOAGOSEI CO., LTD.). The acid value of the polymerizable compound having an acid group is preferably 0.1 to 40 mgKOH/g and more preferably 5 to 30 mgKOH/g. In a case where the acid value of the polymerizable compound is 0.1 mgKOH/g or more, solubility of the coloring composition in a developer is good, and in a case where the acid value of the polymerizable compound is 40 mgKOH/g or less, it is advantageous in production and handling.

The polymerizable compound is preferably a compound having a caprolactone structure. Examples of the polymerizable compound having a caprolactone structure include DPCA-20, DPCA-30, DPCA-60, and DPCA-120, each of which is commercially available as KAYARAD DPCA series from Nippon Kayaku Co., Ltd.

As the polymerizable compound, a polymerizable compound having an alkyleneoxy group can also be used. The polymerizable compound having an alkyleneoxy group is preferably a polymerizable compound having an ethyleneoxy group and/or a propyleneoxy group, more preferably a polymerizable compound having an ethyleneoxy group, and still more preferably a trifunctional to hexafunctional (meth)acrylate compound having 4 to 20 ethyleneoxy groups. Examples of a commercially available product of the polymerizable compound having an alkyleneoxy group include SR-494 manufactured by Sartomer, which is a tetrafunctional (meth)acrylate having 4 ethyleneoxy groups, and KAYARAD TPA-330 manufactured by Nippon Kayaku Co., Ltd., which is a trifunctional (meth)acrylate having 3 isobutyleneoxy groups.

As the polymerizable compound, a polymerizable compound having a fluorene skeleton can also be used. Examples of a commercially available product of the polymerizable compound having a fluorene skeleton include OGSOL EA-0200, EA-0300 (manufactured by Osaka Gas Chemicals Co., Ltd., (meth)acrylate monomer having a fluorene skeleton).

As the polymerizable compound, it is also preferable to use a compound which does not substantially include environmentally regulated substances such as toluene. Examples of a commercially available product of such a compound include KAYARAD DPHA LT and KAYARAD DPEA-12 LT (manufactured by Nippon Kayaku Co., Ltd.).

The urethane acrylates described in JP1973-041708B (JP-S48-041708B), JP1976-037193A (JP-S51-037193A), JP1990-032293B (JP-H02-032293B), or JP1990-016765B (JP-H02-016765B), or the urethane compounds having an ethylene oxide skeleton described in JP1983-049860B (JP-S58-049860B), JP1981-017654B (JP-S56-017654B), JP1987-039417B (JP-S62-039417B), or JP1987-039418B (JP-S62-039418B) are also suitable as the polymerizable compound. In addition, the polymerizable compounds having an amino structure or a sulfide structure in the molecule, described in JP1988-277653A (JP-S63-277653A), JP1988-260909A (JP-S63-260909A), or JP1989-105238A (JP-H01-105238A), are also preferably used. In addition, as the polymerizable compound, commercially available products such as UA-7200 (manufactured by Shin-Nakamura Chemical Co., Ltd.), DPHA-40H (manufactured by Nippon Kayaku Co., Ltd.), and UA-306H, UA-306T, UA-3061, AH-600, T-600, AI-600, and LINC-202UA (manufactured by KYOEISHA CHEMICAL Co., Ltd.) can also be used.

The content of the polymerizable compound in the total solid content of the coloring composition is preferably 0.1% to 50% by mass. The lower limit is more preferably 0.5% by mass or more and still more preferably 1% by mass or more. The upper limit is more preferably 45% by mass or less and still more preferably 40% by mass or less. The polymerizable compound may be used singly or in combination of two or more kinds thereof. In a case where two or more kinds thereof are used in combination, the total thereof is preferably within the above-described range.

<<Photopolymerization Initiator>>

The coloring composition according to the embodiment of the present invention includes a photopolymerization initiator. The photopolymerization initiator is not particularly limited, and can be appropriately selected from known photopolymerization initiators. For example, a compound having photosensitivity to light in a range from an ultraviolet ray range to a visible light range is preferable. The photopolymerization initiator is preferably a photoradical polymerization initiator.

Examples of the photopolymerization initiator include a halogenated hydrocarbon derivative (for example, a compound having a triazine skeleton or a compound having an oxadiazole skeleton), an acylphosphine compound, a hexaarylbiimidazole, an oxime compound, an organic peroxide, a thio compound, a ketone compound, an aromatic onium salt, an a-hydroxyketone compound, and an a-aminoketone compound. From the viewpoint of exposure sensitivity, as the photopolymerization initiator, a trihalomethyltriazine compound, a benzyldimethylketal compound, an a-hydroxyketone compound, an a-aminoketone compound, an acylphosphine compound, a phosphine oxide compound, a metallocene compound, an oxime compound, a triarylimidazole dimer, an onium compound, a benzothiazole compound, a benzophenone compound, an acetophenone compound, a cyclopentadiene-benzene-iron complex, a halomethyl oxadiazole compound, or a 3-aryl-substituted coumarin compound is preferable, a compound selected from an oxime compound, an a-hydroxyketone compound, an a-aminoketone compound, or an acylphosphine compound is more preferable, and an oxime compound is still more preferable. Examples of the photopolymerization initiator include compounds described in paragraphs 0065 to 0111 of JP2014-130173A, and JP6301489B, the contents of which are incorporated herein by reference.

Examples of a commercially available product of the a-hydroxyketone compound include Omnirad 184, Omnirad 1173, Omnirad 2959, and Omnirad 127 (all of which are manufactured by IGM Resins B.V.), Irgacure 184, Irgacure 1173, Irgacure 2959, and Irgacure 127 (all of which are manufactured by BASF). Examples of a commercially available product of the a-aminoketone compound include Omnirad 907, Omnirad 369, Omnirad 369E, and Omnirad 379EG (all of which are manufactured by IGM Resins B.V.), Irgacure 907, Irgacure 369, Irgacure 369E, and Irgacure 379EG (all of which are manufactured by BASF). Examples of a commercially available product of the acylphosphine compound include Omnirad 819 and Omnirad TPO (both of which are manufactured by IGM Resins B.V.), Irgacure 819 and Irgacure TPO (both of which are manufactured by BASF).

Examples of the oxime compound include the compounds described in JP2001-233842A, the compounds described in JP2000-080068A, the compounds described in JP2006-342166A, the compounds described in J. C. S. Perkin II (1979, pp. 1653-1660), the compounds described in J. C. S. Perkin II (1979, pp. 156-162), the compounds described in Journal of Photopolymer Science and Technology (1995, pp. 202-232), the compounds described in JP2000-066385A, the compounds described in JP2004-534797A, the compounds described in JP2006-342166A, the compounds described in JP2017-019766A, the compounds described in JP6065596B, the compounds described in WO2015/152153A, the compounds described in WO2017/051680A, the compounds described in JP2017-198865A, the compounds described in paragraph Nos. 0025 to 0038 of WO2017/164127A, and the compounds described in WO2013/167515A. Specific examples of the oxime compound include 3-benzoyloxyiminobutane-2-one, 3-acetoxyiminobutane-2-one, 3-propionyloxyiminobutane-2-one, 2-acetoxyiminopentane-3 -one, 2-acetoxyimino-1-phenylpropane-1-one, 2-benzoyloxyimino-1-phenylpropane-1-one, 3-(4-toluene sulfonyloxy)iminobutane-2-one, and 2-ethoxycarbonyloxyimino-1-phenylpropane-1-one. Examples of a commercially available product thereof include Irgacure OXE01, Irgacure OXE02, Irgacure OXE03, and Irgacure OXE04 (all of which are manufactured by BASF), TR-PBG-304 (manufactured by TRONLY), and ADEKA OPTOMER N-1919 (manufactured by ADEKA Corporation; photopolymerization initiator 2 described in JP2012-014052A). In addition, as the oxime compound, it is also preferable to use a compound having no colorability or a compound having high transparency and being resistant to discoloration. Examples of a commercially available product include ADEKA ARKLS NCI-730, NCI-831, and NCI-930 (all of which are manufactured by ADEKA Corporation).

In addition, an oxime compound having a fluorene ring can also be used as the photopolymerization initiator. Specific examples of the oxime compound having a fluorene ring include the compounds described in JP2014-137466A.

In addition, as the photopolymerization initiator, an oxime compound having a skeleton in which at least one benzene ring of a carbazole ring is a naphthalene ring can also be used. Specific examples of such an oxime compound include the compounds described in WO2013/083505A.

In addition, an oxime compound having a fluorine atom can also be used as the photopolymerization initiator. Specific examples of the oxime compound having a fluorine atom include the compounds described in JP2010-262028A, the compounds 24, and 36 to 40 described in JP2014-500852A, and the compound (C-3) described in JP2013-164471A.

In the present invention, an oxime compound having a nitro group can be used as the photopolymerization initiator. The oxime compound having a nitro group is also preferably used in the form of a dimer. Specific examples of the oxime compound having a nitro group include the compounds described in paragraph Nos. 0031 to 0047 of JP2013-114249A and paragraph Nos. 0008 to 0012 and 0070 to 0079 of JP2014-137466A, the compounds described in paragraph Nos. 0007 to 0025 of JP4223071B, and ADEKA ARKLS NCI-831 (manufactured by ADEKA Corporation).

In addition, an oxime compound having a benzofuran skeleton can also be used as the photopolymerization initiator. Specific examples thereof include OE-01 to OE-75 described in WO2015/036910A.

Specific examples of the oxime compound which are preferably used in the present invention are shown below, but the present invention is not limited thereto.

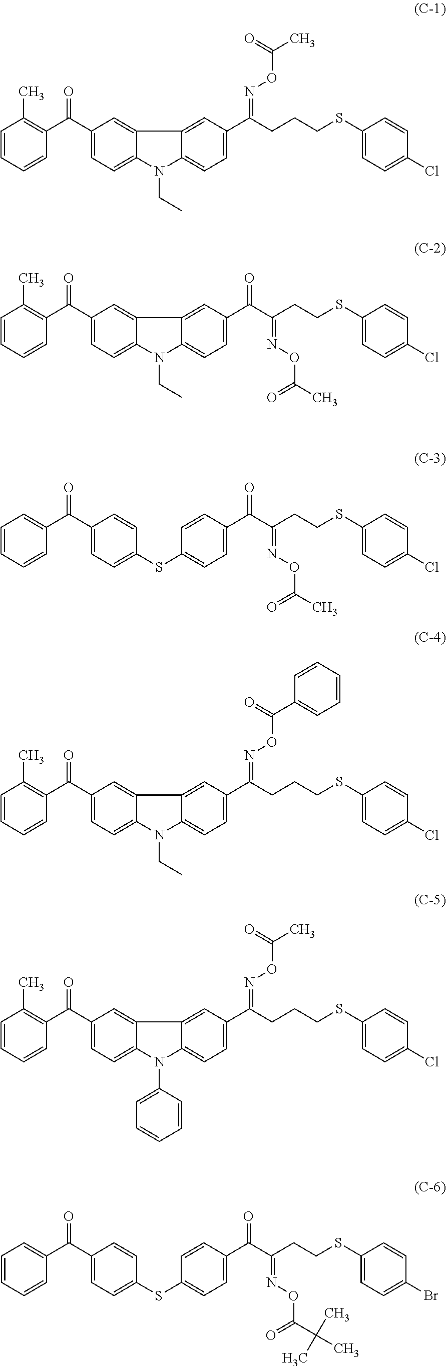 [SMILES: CCn1c2ccc(C(=O)c3ccccc3C)cc2c2cc(/C(CCCSc3ccc(Cl)cc3)=N/OC(C)=O)ccc21 ; CCn1c2ccc(C(=O)/C(CCSc3ccc(Cl)cc3)=N/OC(C)=O)cc2c2cc(C(=O)c3ccccc3C)ccc21 ; CC(=O)O/N=C(\CCSc1ccc(Cl)cc1)C(=O)c1ccc(Sc2ccc(C(=O)c3ccccc3)cc2)cc1 ; CCn1c2ccc(C(=O)c3ccccc3C)cc2c2cc(/C(CCCSc3ccc(Cl)cc3)=N/OC(=O)c3ccccc3)ccc21 ; CC(=O)O/N=C(\CCCSc1ccc(Cl)cc1)c1ccc2c(c1)c1cc(C(=O)c3ccccc3C)ccc1n2-c1ccccc1 ; CC(C)(C)C(=O)O/N=C(\CCSc1ccc(Br)cc1)C(=O)c1ccc(Sc2ccc(C(=O)c3ccccc3)cc2)cc1]

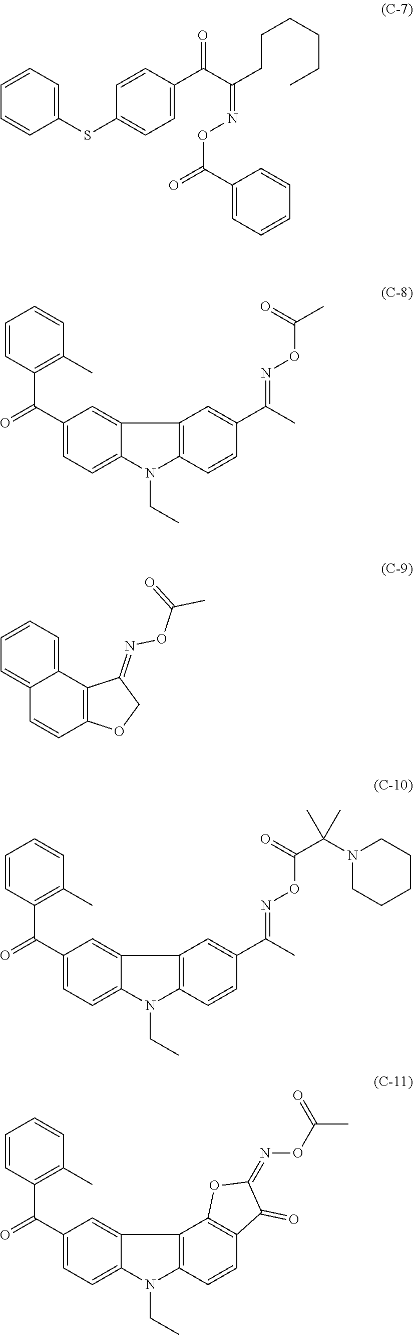 [SMILES: CCCCCC/C(=N/OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21 ; CC(=O)O/N=C1\COc2ccc3ccccc3c21 ; CCn1c2ccc(C(=O)c3ccccc3C)cc2c2cc(/C(C)=N/OC(=O)C(C)(C)N3CCCCC3)ccc21 ; CCn1c2ccc(C(=O)c3ccccc3C)cc2c2c3c(ccc21)C(=O)/C(=N\OC(C)=O)O3]

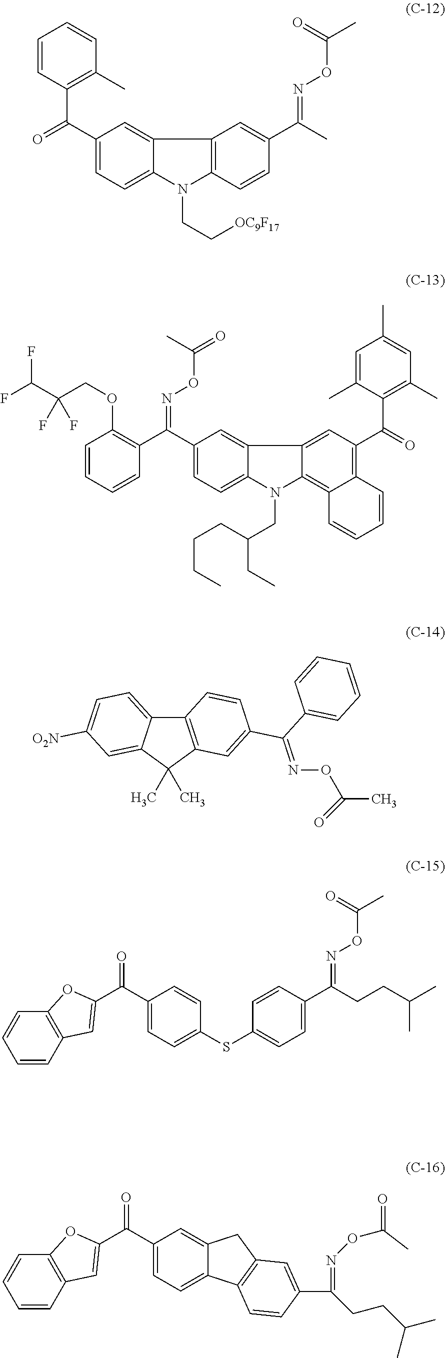

The oxime compound is preferably a compound having a maximal absorption wavelength in a wavelength range of 350 to 500 nm and more preferably a compound having a maximal absorption wavelength in a wavelength range of 360 to 480 nm. In addition, from the viewpoint of sensitivity, the molar absorption coefficient of the oxime compound at a wavelength of 365 nm or 405 nm is preferably high, more preferably 1000 to 300000, still more preferably 2000 to 300000, and particularly preferably 5000 to 200000. The molar absorption coefficient of a compound can be measured using a well-known method. For example, it is preferable that the molar absorption coefficient can be measured using a spectrophotometer (Cary-5 spectrophotometer, manufactured by Varian Medical Systems, Inc.) and ethyl acetate at a concentration of 0.01 g/L.

As the photopolymerization initiator, a bifunctional or tri- or higher functional photoradical polymerization initiator may be used. By using such a photoradical polymerization initiator, two or more radicals are generated from one molecule of the photoradical polymerization initiator, and as a result, good sensitivity is obtained. In addition, in a case of using a compound having an asymmetric structure, crystallinity is reduced so that solubility in a solvent or the like is improved, precipitation is to be difficult over time, and temporal stability of the coloring composition can be improved. Specific examples of the bifunctional or tri- or higher functional photoradical polymerization initiator include dimers of the oxime compounds described in JP2010-527339A, JP2011-524436A, WO2015/004565A, paragraph Nos. 0407 to 0412 of JP2016-532675A, and paragraph Nos. 0039 to 0055 of WO2017/033680A; the compound (E) and compound (G) described in JP2013-522445A; Cmpd 1 to 7 described in WO2016/034963A; the oxime ester photoinitiators described in paragraph No. 0007 of JP2017-523465A; the photoinitiators described in paragraph Nos. 0020 to 0033 of JP2017-167399A; the photopolymerization initiator (A) described in paragraph Nos. 0017 to 0026 of JP2017-151342A; and the oxime ester photoinitiators described in JP6469669B.

The content of the photopolymerization initiator in the total solid content of the coloring composition according to the embodiment of the present invention is preferably 0.1% to 30% by mass. The lower limit is preferably 0.5% by mass or more and more preferably 1% by mass or more. The upper limit is preferably 20% by mass or less and more preferably 15% by mass or less. In the coloring composition according to the embodiment of the present invention, the photopolymerization initiator may be used singly or in combination of two or more kinds thereof. In a case where two or more kinds thereof are used, the total amount thereof is preferably within the above-described range.

<<Resin>>

The coloring composition according to the embodiment of the present invention can contain a resin. The resin is blended in, for example, an application for dispersing the pigment in the coloring composition or an application as a binder. Mainly, a resin which is used for dispersing the pigment is also referred to as a dispersant. However, such applications of the resin are only exemplary, and the resin can also be used for other purposes in addition to such applications.

Examples of the resin include a (meth)acrylic resin, a (meth)acrylamide resin, an epoxy resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamide resin, a polyolefin resin, a cyclic olefin resin, a polyester resin, a styrene resin, a siloxane resin, a polyimine resin, and a polyurethane resin.

The weight-average molecular weight (Mw) of the resin is preferably 3000 to 2000000. The upper limit is preferably 1000000 or less and more preferably 500000 or less. The lower limit is preferably 4000 or more and more preferably 5000 or more.

The coloring composition according to the embodiment of the present invention preferably includes an alkali-soluble resin. In a case where the coloring composition according to the embodiment of the present invention includes an alkali-soluble resin, developability of the coloring composition is improved, and in a case where a pattern is formed by a photolithography method using the coloring composition according to the embodiment of the present invention, the generation of development residue and the like can be effectively suppressed. Examples of the alkali-soluble resin include resins having an acid group. Examples of the acid group include a carboxyl group, a phosphoric acid group, a sulfo group, and a phenolic hydroxy group, and a carboxyl group is preferable. The acid group included in the alkali-soluble resin may be used singly or in combination of two or more kinds thereof. The alkali-soluble resin can also be used as a dispersant.

The alkali-soluble resin preferably includes a repeating unit having an acid group in the side chain, and more preferably includes 5% to 70% by mole of repeating units having an acid group in the side chain with respect to the total repeating units of the resin. The upper limit of the content of the repeating unit having an acid group in the side chain is preferably 50 mol % or less and more preferably 30 mol % or less. The lower limit of the content of the repeating unit having an acid group in the side chain is preferably 10% by mole or more and more preferably 20% by mole or more.

The alkali-soluble resin is also preferably an alkali-soluble resin having a polymerizable group. Examples of the polymerizable group include a (meth)allyl group and a (meth)acryloyl group. The alkali-soluble resin having a polymerizable group is preferably a resin including a repeating unit having a polymerizable group in the side chain and a repeating unit having an acid group in the side chain.

It is also preferable that the alkali-soluble resin includes a repeating unit derived from a monomer component including a compound represented by Formula (ED1) and/or a compound represented by Formula (ED2) (hereinafter, these compounds may be referred to as an “ether dimer”).

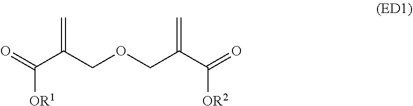

In Formula (ED1), R¹ and R² each independently represent a hydrogen atom or a hydrocarbon group having 1 to 25 carbon atoms, which may have a substituent.

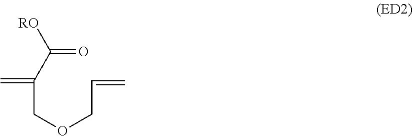

In Formula (ED2), R represents a hydrogen atom or an organic group having 1 to 30 carbon atoms. With regard to details of Formula (ED2), reference can be made to the description in JP2010-168539A, the contents of which are incorporated herein by reference.

With regard to the specific examples of the ether dimer, reference can be made to the description in paragraph No. 0317 of JP2013-029760A, the contents of which are incorporated herein by reference.

With regard to the alkali-soluble resin, reference can be made to the description in paragraph Nos. 0558 to 0571 of JP2012-208494A (paragraph Nos. 0685 to 0700 of the corresponding US2012/0235099A), the description in paragraph Nos. 0076 to 0099 of JP2012-198408A, and the description of JP2018-105911A, the contents of which are incorporated herein by reference.

The acid value of the alkali-soluble resin is preferably 30 to 500 mgKOH/g. The lower limit is preferably 50 mgKOH/g or more and more preferably 70 mgKOH/g or more. The upper limit is preferably 400 mgKOH/g or less, more preferably 300 mgKOH/g or less, and still more preferably 200 mgKOH/g or less. The weight-average molecular weight (Mw) of the alkali-soluble resin is preferably 5000 to 100,000.

The coloring composition according to the embodiment of the present invention also preferably includes a resin (hereinafter, also referred to as a resin Ac) having an aromatic carboxyl group. By using the resin Ac, a network of pigment-triazine compound (TA)-resin Ac is easily formed in the film, and aggregation of the pigment in the film can be effectively suppressed, so that it is possible to form a film having less color unevenness. The resin Ac is also the alkali-soluble resin.

The resin Ac may include the aromatic carboxyl group in the main chain of the repeating unit, or in the side chain of the repeating unit. From the reason that the effects described above can be easily obtained more remarkably, it is preferable that the aromatic carboxyl group is included in the main chain of the repeating unit. The details are not clear, but it is presumed that the presence of the aromatic carboxyl group near the main chain further improves these properties. In the present specification, the aromatic carboxyl group is a group having a structure in which one or more carboxyl groups are bonded to an aromatic ring. In the aromatic carboxyl group, the number of carboxyl groups bonded to an aromatic ring is preferably 1 to 4 and more preferably 1 or 2.

The resin Ac is preferably a resin including at least one repeating unit selected from a repeating unit represented by Formula (b-1) or a repeating unit represented by Formula (b-10).

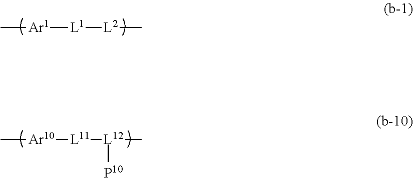

In Formula (b-1), Ar¹ represents a group including an aromatic carboxyl group, L¹ represents —COO— or —CONH—, and L² represents a divalent linking group.

In Formula (b-10), Ar¹⁰ represents a group including an aromatic carboxyl group, L¹¹ represents —COO— or —CONH—, L¹² represents a trivalent linking group, and P¹⁰ represents a polymer chain.

First, Formula (b-1) will be described. In Formula (b-1), examples of the group including an aromatic carboxyl group, represented by Ar¹, include a structure derived from an aromatic tricarboxylic acid anhydride and a structure derived from an aromatic tetracarboxylic acid anhydride. Examples of the aromatic tricarboxylic acid anhydride and the aromatic tetracarboxylic acid anhydride include compounds having the following structures.

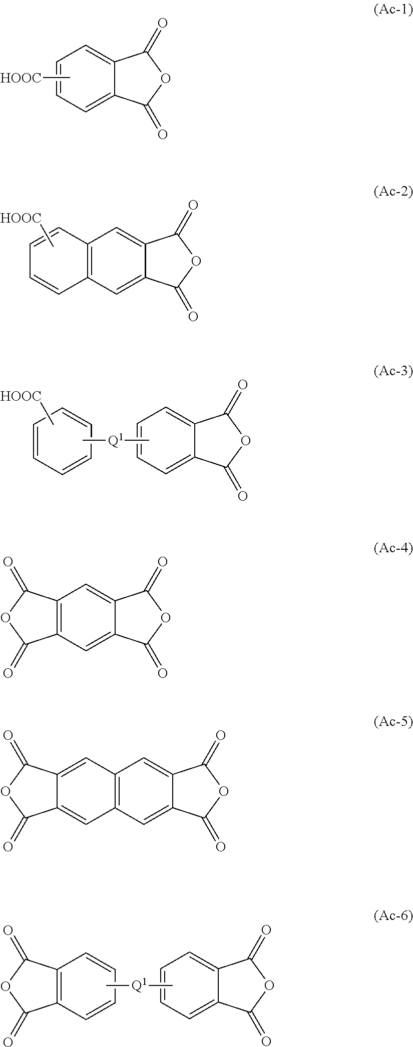

In the formulae, Q¹ represents a single bond, —O—, —CO—, —COOCH₂CH₂OCO—, —SO₂—, —C(CF₃)₂—, a group represented by Formula (Q-1), or a group represented by Formula (Q-2).

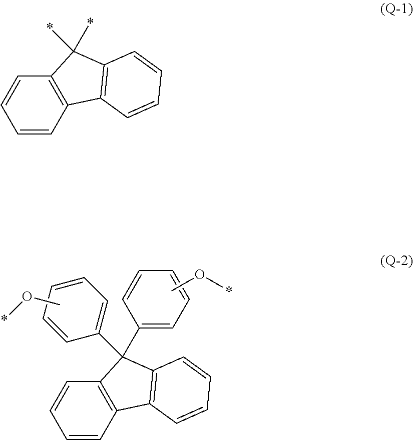

Specific examples of the group including an aromatic carboxyl group represented by Ar¹ include a group represented by Formula (Ar-1), a group represented by Formula (Ar-2), and a group represented by Formula (Ar-3).

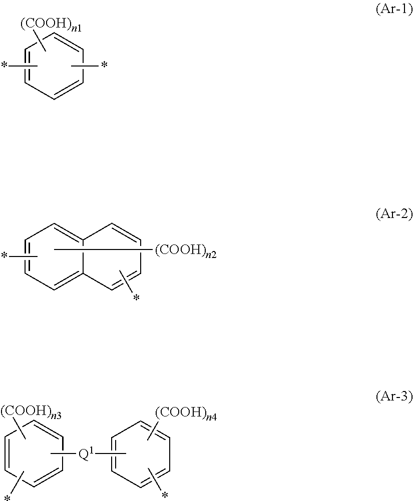

In Formula (Ar-1), n1 represents an integer of 1 to 4, and is preferably 1 or 2 and more preferably 2.

In Formula (Ar-2), n2 represents an integer of 1 to 8, and is preferably an integer of 1 or 4, more preferably 1 or 2, and still more preferably 2.

In Formula (Ar-3), n3 and n4 each independently represent an integer of 0 to 4, and are preferably an integer of 0 or 2, more preferably 1 or 2, and still more preferably 1. However, at least one of n3 or n4 is an integer of 1 or more.

In Formula (Ar-3), Q¹ represents a single bond, —O—, —CO—, —COOCH₂CH₂OCO—, —SO₂—, —C(CF₃)₂—, the above-described group represented by Formula (Q-1), or the above-described group represented by Formula (Q-2).

In Formula (b-1), L¹ represents —COO— or —CONH—, preferably —COO—.

In Formula (b-1), examples of the divalent linking group represented by L² include an alkylene group, an arylene group, —O—, —CO—, —COO——OCO—, —NH—, —S—, and a group formed by a combination of two or more of these groups. The number of carbon atoms in the alkylene group preferably is 1 to 30, more preferably 1 to 20, and still more preferably 1 to 15. The alkylene group may be linear, branched, or cyclic. The number of carbon atoms in the arylene group is preferably 6 to 30, more preferably 6 to 20, and still more preferably 6 to 10. The alkylene group and the arylene group may have a substituent. Examples of the substituent include a hydroxy group. The divalent linking group represented by L² is preferably a group represented by —O-L^{2a}-O—. Examples of L^{2a} include an alkylene group; an arylene group; a group formed by a combination of an alkylene group and an arylene group; and a group formed by a combination of at least one selected from an alkylene group or an arylene group, and at least one selected from —O—, —CO—, —COO——OCO—, —NH—, or —S—. The number of carbon atoms in the alkylene group preferably is 1 to 30, more preferably 1 to 20, and still more preferably 1 to 15. The alkylene group may be linear, branched, or cyclic. The alkylene group and the arylene group may have a substituent. Examples of the substituent include a hydroxy group.

Next, Formula (b-10) will be described. In Formula (b-10), the group including an aromatic carboxyl group, represented by Ar¹⁰, has the same meaning as Ar¹ in Formula (b-1), and the preferred range is also the same.

In Formula (b-10), L¹¹ represents —COO— or —CONH—, preferably —COO—.

In Formula (b-10), examples of the trivalent linking group represented by L¹² include a hydrocarbon group, —O—, —CO—, —COO——OCO—, —NH—, —S—, and a group formed by a combination of two or more of these groups. Examples of the hydrocarbon group include an aliphatic hydrocarbon group and an aromatic hydrocarbon group. The number of carbon atoms in the aliphatic hydrocarbon group is preferably 1 to 30, more preferably 1 to 20, and still more preferably 1 to 15. The aliphatic hydrocarbon group may be linear, branched, or cyclic. The number of carbon atoms in the aromatic hydrocarbon group is preferably 6 to 30, more preferably 6 to 20, and still more preferably 6 to 10. The hydrocarbon group may have a substituent. Examples of the substituent include a hydroxy group.

In Formula (b-10), P¹⁰ represents a polymer chain. It is preferable that the polymer chain represented by P¹⁰ has at least one repeating unit selected from a poly(meth)acrylic repeating unit, a polyether repeating unit, a polyester repeating unit, or a polyol repeating unit. The weight-average molecular weight of the polymer chain P¹⁰ is preferably 500 to 20000. The lower limit is preferably 1000 or more. The upper limit is preferably 10000 or less, more preferably 5000 or less, and still more preferably 3000 or less. In a case where the weight-average molecular weight of P¹⁰ is within the above-described range, dispersibility of the pigment in the composition is good. In a case where the resin having an aromatic carboxyl group is a resin having the repeating unit represented by Formula (b-10), this resin is preferably used as a dispersant.

The weight-average molecular weight of the resin Ac is preferably 2000 to 35000. The upper limit is preferably 25000 or less and more preferably 15000 or less. The lower limit is preferably 4000 or more and more preferably 7000 or more.

The acid value of the resin Ac is preferably 5 to 200 mgKOH/g. The lower limit is preferably 10 mgKOH/g or more, more preferably 15 mgKOH/g or more, and still more preferably 20 mgKOH/g or more. The upper limit is preferably 150 mgKOH/g or less and more preferably 100 mgKOH/g or less.

The coloring composition according to the embodiment of the present invention can also include a resin as a dispersant. Examples of the dispersant include an acidic dispersant (acidic resin) and a basic dispersant (basic resin). Here, the acidic dispersant (acidic resin) represents a resin in which the amount of the acid group is larger than the amount of the basic group. The acidic dispersant (acidic resin) is preferably a resin in which the amount of the acid group occupies 70% by mole or more in a case where the total amount of the acid group and the basic group is 100% by mole, and more preferably a resin substantially consisting of only an acid group. The acid group included in the acidic dispersant (acidic resin) is preferably a carboxyl group. The acid value of the acidic dispersant (acidic resin) is preferably 40 to 105 mgKOH/g, more preferably 50 to 105 mgKOH/g, and still more preferably 60 to 105 mgKOH/g. In addition, the basic dispersant (basic resin) represents a resin in which the amount of the basic group is larger than the amount of the acid group. The basic dispersant (basic resin) is preferably a resin in which the amount of the basic group is more than 50% by mole in a case where the total amount of the acid group and the basic group is 100% by mole. The basic group included in the basic dispersant is preferably an amino group.

The resin used as a dispersant preferably includes a repeating unit having an acid group. In a case where the resin used as a dispersant includes a repeating unit having an acid group, the generation of the development residue can be further suppressed in the formation of a pattern by a photolithography method.

It is also preferable that the resin used as a dispersant is a graft resin. With regard to details of the graft resin, reference can be made to the description in paragraph Nos. 0025 to 0094 of JP2012-255128A, the contents of which are incorporated herein by reference.

It is also preferable that the resin used as a dispersant is a polyimine-based dispersant including a nitrogen atom in at least one of the main chain or the side chain. As the polyimine-based dispersant, a resin having a main chain which has a partial structure having a functional group of pKa14 or less, and a side chain which has 40 to 10000 atoms, in which at least one of the main chain or the side chain has a basic nitrogen atom, is preferable. The basic nitrogen atom is not particularly limited as long as it is a nitrogen atom exhibiting basicity. With regard to the polyimine-based dispersant, reference can be made to the description in paragraph Nos. 0102 to 0166 of JP2012-255128A, the contents of which are incorporated herein by reference.

It is also preferable that the resin used as a dispersant is a resin having a structure in which a plurality of polymer chains are bonded to a core portion. Examples of such a resin include dendrimers (including star polymers). In addition, specific examples of the dendrimer include polymer compounds C-1 to C-31 described in paragraph Nos. 0196 to 0209 of JP2013-043962A.

In addition, the above-described resin Ac can also be used as a dispersant.

It is also preferable that the resin used as a dispersant are a resin including a repeating unit having an ethylenically unsaturated bond-containing group in the side chain. The content of the repeating unit having an ethylenically unsaturated bond-containing group in the side chain is preferably 10 mol % or more, more preferably 10 to 80 mol %, and still more preferably 20 to 70 mol % with respect to the total repeating units of the resin. In addition, as the dispersant, a resin described in JP2018-087939A can also be used.

A commercially available product is also available as the dispersant, and specific examples thereof include DISPERBYK series manufactured by BYK Chemie Japan, Solsperse series manufactured by Lubrizol Japan Ltd., Efka series manufactured by BASF, and AJISPER series manufactured by Ajinomoto Fine-Techno Co., Inc. In addition, products described in paragraph No. 0129 of JP2012-137564A and products described in paragraph No. 0235 of JP2017-194662A can also be used as the dispersant. In addition, as the dispersant, compounds described in JP2018-150498A, JP2017-100116A, JP2017-100115A, JP2016-108520A, JP2016-108519A, and JP2015-232105A may be used.

In a case where the coloring composition according to the embodiment of the present invention includes a resin, the content of the resin in the total solid content of the coloring composition is preferably 5% to 50% by mass. The lower limit is preferably 10% by mass or more and more preferably 15% by mass or more. The upper limit is preferably 40% by mass or less, more preferably 35% by mass or less, and still more preferably 30% by mass or less.

In addition, the content of the resin having an acid group in the total solid content of the coloring composition is preferably 5% to 50% by mass. The lower limit is preferably 10% by mass or more and more preferably 15% by mass or more. The upper limit is preferably 40% by mass or less, more preferably 35% by mass or less, and still more preferably 30% by mass or less. In addition, from the reason that excellent developability is easily obtained, the content of the resin having an acid group in the total amount of the resin is preferably 30% by mass or more, more preferably 50% by mass or more, still more preferably 70% by mass or more, and particularly preferably 80% by mass or more. The upper limit may be 100% by mass, 95% by mass, or 90% by mass or less.

In addition, the content of the alkali-soluble resin in the total solid content of the coloring composition is preferably 5% to 50% by mass. The lower limit is preferably 10% by mass or more and more preferably 15% by mass or more. The upper limit is preferably 40% by mass or less, more preferably 35% by mass or less, and still more preferably 30% by mass or less. In addition, from the reason that excellent developability is easily obtained, the content of the alkali-soluble resin in the total amount of the resin is preferably 30% by mass or more, more preferably 50% by mass or more, still more preferably 70% by mass or more, and particularly preferably 80% by mass or more. The upper limit may be 100% by mass, 95% by mass, or 90% by mass or less.

In addition, the content of the resin Ac in the total solid content of the coloring composition is preferably 1% to 50% by mass. The lower limit is preferably 5% by mass or more and more preferably 10% by mass or more. The upper limit is preferably 45% by mass or less, more preferably 40% by mass or less, and still more preferably 35% by mass or less. In addition, the content of the resin Ac in the total amount of the resin is preferably 50% by mass or more, more preferably 60% by mass or more, and still more preferably 70% by mass or more. The upper limit may be 100% by mass, 99% by mass or less, 95% by mass or less, or 90% by mass or less.

In addition, from the viewpoint of curing properties, developability, and film-forming property, the total content of the polymerizable compound and resin in the total solid content of the coloring composition is preferably 10% to 65% by mass. The lower limit is preferably 15% by mass or more, more preferably 20% by mass or more, and still more preferably 30% by mass or more. The upper limit is preferably 60% by mass or less, more preferably 50% by mass or less, and still more preferably 40% by mass or less. In addition, the coloring composition according to the embodiment of the present invention preferably contains 30 to 300 parts by mass of the resin with respect to 100 parts by mass of the polymerizable compound. The lower limit is preferably 50 parts by mass or more and more preferably 80 parts by mass or more. The upper limit is preferably 250 parts by mass or less and more preferably 200 parts by mass or less.

<<Compound Having Cyclic Ether Group>>

The coloring composition according to the embodiment of the present invention can contain a compound having a cyclic ether group. Examples of the cyclic ether group include an epoxy group and an oxetanyl group. The compound having a cyclic ether group is preferably a compound having an epoxy group. Examples of the compound having an epoxy group include a compound having one or more epoxy groups in one molecule, and a compound having two or more epoxy groups in one molecule is preferable. It is preferable to have 1 to 100 epoxy groups in one molecule. The upper limit of the number of epoxy groups may be, for example, 10 or less or 5 or less. The lower limit of the number of epoxy groups is preferably 2 or more. As the compound having an epoxy group, the compounds described in paragraph Nos. 0034 to 0036 of JP2013-011869A, paragraph Nos. 0147 to 0156 of JP2014-043556A, and paragraph Nos. 0085 to 0092 of JP2014-089408A, and the compounds described in JP2017-179172A can also be used. The contents of the publications are incorporated herein by reference.

The compound having an epoxy group may be a low-molecular-weight compound (for example, a molecular weight of less than 1000) or a high-molecular-weight compound (macromolecule) (for example, a molecular weight of 1000 or more, and in a case of a polymer, a weight-average molecular weight of 1000 or more). The weight-average molecular weight of the compound having an epoxy group is preferably 200 to 100000 and more preferably 500 to 50000. The upper limit of the weight-average molecular weight is preferably 10000 or less, more preferably 5000 or less, and still more preferably 3000 or less.

As the compound having an epoxy group, an epoxy resin can be preferably used. Examples of the epoxy resin include an epoxy resin which is a glycidyl etherified product of a phenol compound, an epoxy resin which is a glycidyl etherified product of various novolak resins, an alicyclic epoxy resin, an aliphatic epoxy resin, a heterocyclic epoxy resin, a glycidyl ester-based epoxy resin, a glycidyl amine-based epoxy resin, an epoxy resin obtained by glycidylating halogenated phenols, a condensate of a silicon compound having an epoxy group and another silicon compound, and a copolymer of a polymerizable unsaturated compound having an epoxy group and another polymerizable unsaturated compound. The epoxy equivalent of the epoxy resin is preferably 310 to 3300 g/eq, more preferably 310 to 1700 g/eq, and still more preferably 310 to 1000 g/eq.

Examples of a commercially available product of the compound having a cyclic ether group include EHPE 3150 (manufactured by Daicel Corporation), EPICLON N-695 (manufactured by DIC Corporation), and MARPROOF G-0150M, G-0105SA, G-0130SP, G-0250SP, G-1005S, G-1005SA, G-1010S, G-2050M, G-01100, and G-01758 (all of which are manufactured by NOF Corporation., an epoxy group-containing polymer).

In a case where the coloring composition according to the embodiment of the present invention contains a compound having a cyclic ether group, the content of the compound having a cyclic ether group in the total solid content of the coloring composition is preferably 0.1% to 20% by mass. The lower limit is, for example, preferably 0.5% by mass or more and more preferably 1% by mass or more. The upper limit is, for example, preferably 15% by mass or less and still more preferably 10% by mass or less. The compound having a cyclic ether group may be used singly or in combination of two or more kinds thereof. In a case of using two or more kinds thereof, the total amount thereof is preferably within the above-described range.

<<Silane Coupling Agent>>

The coloring composition according to the embodiment of the present invention can contain a silane coupling agent. According to this aspect, adhesiveness of a film to be obtained with a support can be further improved. In the present invention, the silane coupling agent means a silane compound having a hydrolyzable group and other functional groups. In addition, the hydrolyzable group refers to a substituent directly linked to a silicon atom and capable of forming a siloxane bond due to at least one of a hydrolysis reaction or a condensation reaction. Examples of the hydrolyzable group include a halogen atom, an alkoxy group, and an acyloxy group, and an alkoxy group is preferable. That is, it is preferable that the silane coupling agent is a compound having an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, an isocyanate group, and a phenyl group, and an amino group, a (meth)acryloyl group, or an epoxy group is preferable. Specific examples of the silane coupling agent include N-β-aminoethyl-γ-aminopropyl methyldimethoxysilane (trade name: KBM-602, manufactured by Shin-Etsu Chemical Co., Ltd.), N-β-aminoethyl-γ-aminopropyl trimethoxysilane (trade name: KBM-603, manufactured by Shin-Etsu Chemical Co., Ltd.), N-β-aminoethyl-y-aminopropyl triethoxysilane (trade name: KBE-602, manufactured by Shin-Etsu Chemical Co., Ltd.), y-aminopropyl trimethoxysilane (trade name: KBM-903, manufactured by Shin-Etsu Chemical Co., Ltd.), y-aminopropyl triethoxysilane (trade name: KBE-903, manufactured by Shin-Etsu Chemical Co., Ltd.), 3-methacryloxypropyl methyldimethoxysilane (trade name: KBM-502, manufactured by Shin-Etsu Chemical Co., Ltd.), and 3-methacryloxypropyl trimethoxysilane (trade name: KBM-503, manufactured by Shin-Etsu Chemical Co., Ltd.). In addition, specific examples of the silane coupling agent include the compounds described in paragraph Nos. 0018 to 0036 of JP2009-288703A and the compounds described in paragraph Nos. 0056 to 0066 of JP2009-242604A, the contents of which are incorporated herein by reference.

The content of the silane coupling agent in the total solid content of the coloring composition is preferably 0.1% to 5% by mass. The upper limit is preferably 3% by mass or less and more preferably 2% by mass or less. The lower limit is preferably 0.5% by mass or more and more preferably 1% by mass or more. The silane coupling agent may be used singly or in combination of two or more kinds thereof. In a case of using two or more kinds thereof, the total amount thereof is preferably within the above-described range.

<<Solvent>>

The coloring composition according to the embodiment of the present invention can contain a solvent. Examples of the solvent include an organic solvent. Basically, the solvent is not particularly limited as long as it satisfies the solubility of the respective components and the application properties of the coloring composition. Examples of the organic solvent include an ester-based solvent, a ketone-based solvent, an alcohol-based solvent, an amide-based solvent, an ether-based solvent, and a hydrocarbon-based solvent. With regard to details thereof, reference can be made to the description in paragraph No. 0223 of WO2015/166779A, the contents of which are incorporated herein by reference. In addition, an ester-based solvent substituted with a cyclic alkyl group or a ketone-based solvent substituted with a cyclic alkyl group can also be preferably used. Specific examples of the organic solvent include polyethylene glycol monomethyl ether, dichloromethane, methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclohexyl acetate, cyclopentanone, ethyl carbitol acetate, butyl carbitol acetate, propylene glycol monomethyl ether, propylene glycol monomethyl ether acetate, 3-methoxy-N,N-dimethylpropanamide, and 3-butoxy-N,N-dimethylpropanamide. In this case, it may be preferable that the content of aromatic hydrocarbons (such as benzene, toluene, xylene, and ethylbenzene) as the solvent is low (for example, 50 parts per million (ppm) by mass or less, 10 ppm by mass or less, or 1 ppm by mass or less with respect to the total amount of the organic solvent) in consideration of environmental aspects and the like.

In the present invention, a solvent having a low metal content is preferably used. For example, the metal content in the solvent is preferably 10 ppb (parts per billion) by mass or less. A solvent in which the metal content is at a level of ppt (parts per trillion) by mass may be used as desired, and such a high-purity solvent is provided by, for example, Toyo Kasei Kogyo Co., Ltd. (The Chemical Daily, Nov. 13, 2015).

Examples of a method for removing impurities such as a metal from the solvent include distillation (such as molecular distillation and thin-film distillation) and filtration using a filter. The filter pore size of the filter used for the filtration is preferably 10 μm or less, more preferably 5 μm or less, and still more preferably 3 μm or less. As a material of the filter, polytetrafluoroethylene, polyethylene, or nylon is preferable.

The solvent may include isomers (compounds having the same number of atoms and different structures). In addition, only one kind of isomers may be included, or a plurality of isomers may be included.

In the present invention, the organic solvent preferably has the content of peroxides of 0.8 mmol/L or less, and more preferably, the organic solvent does not substantially include peroxides.

The content of the solvent in the coloring composition is preferably 10% to 95% by mass, more preferably 20% to 90% by mass, and still more preferably 30% to 90% by mass.

In addition, from the viewpoint of environmental regulation, it is preferable that the coloring composition according to the embodiment of the present invention does not substantially contain environmentally regulated substances. In the present invention, the description “does not substantially contain environmentally regulated substances” means that the content of the environmentally regulated substances in the coloring composition is 50 ppm by mass or less, preferably 30 ppm by mass or less, still more preferably 10 ppm by mass or less, and particularly preferably 1 ppm by mass or less. Examples of the environmentally regulated substances include benzenes; alkylbenzenes such as toluene and xylene; and halogenated benzenes such as chlorobenzene. These compounds are registered as environmentally regulated substances in accordance with Registration Evaluation Authorization and Restriction of Chemicals (REACH) rules, Pollutant Release and Transfer Register (PRTR) law, Volatile Organic Compounds (VOC) regulation, and the like, and strictly regulated in their usage and handling method. These compounds can be used as a solvent in a case of producing respective components used in the coloring composition according to the embodiment of the present invention, and may be incorporated into the coloring composition as a residual solvent. From the viewpoint of human safety and environmental considerations, it is preferable to reduce these substances as much as possible. Examples of a method for reducing the environmentally regulated substances include a method for reducing the environmentally regulated substances by distilling the environmentally regulated substances from a system by heating or depressurizing the system such that the temperature of the system is higher than a boiling point of the environmentally regulated substances. In addition, in a case of distilling a small amount of the environmentally regulated substances, it is also useful to azeotrope with a solvent having the boiling point equivalent to that of the above-described solvent in order to increase efficiency. In addition, in a case of containing a compound having radical polymerizability, in order to suppress the radical polymerization reaction proceeding during the distillation under reduced pressure to cause crosslinking between the molecules, a polymerization inhibitor or the like may be added and the distillation under reduced pressure is performed. These distillation methods can be performed at any stage of raw material, product (for example, resin solution after polymerization or polyfunctional monomer solution) obtained by reacting the raw material, coloring composition produced by mixing these compounds, or the like.

<<Polymerization Inhibitor>>

The coloring composition according to the embodiment of the present invention can contain a polymerization inhibitor. Examples of the polymerization inhibitor include hydroquinone, p-methoxyphenol, di-tert-butyl-p-cresol, pyrogallol, tert-butyl catechol, benzoquinone, 4,4′-thiobis(3-methyl-6-tert-butylphenol), 2,2′-methylenebis(4-methyl-6-t-butylphenol), and an N-nitrosophenylhydroxylamine salt (an ammonium salt, a cerous salt, or the like). Among these, p-methoxyphenol is preferable. The content of the polymerization inhibitor in the total solid content of the coloring composition is preferably 0.0001% to 5% by mass.

<<Surfactant>>

The coloring composition according to the embodiment of the present invention can contain a surfactant. As the surfactant, various surfactants such as a fluorine-based surfactant, a nonionic surfactant, a cationic surfactant, an anionic surfactant, and a silicon-based surfactant can be used. Examples of the surfactant include surfactants described in paragraph Nos. 0238 to 0245 of WO2015/166779A, the contents of which are incorporated herein by reference.

In the present invention, it is preferable that the surfactant is a fluorine-based surfactant. By containing a fluorine-based surfactant in the coloring composition, liquid characteristics (particularly, fluidity) are further improved, and liquid saving properties can be further improved. In addition, it is possible to form a film with a small thickness unevenness.

The fluorine content in the fluorine-based surfactant is suitably 3% to 40% by mass, and more preferably 5% to 30% by mass and particularly preferably 7% to 25% by mass. The fluorine-based surfactant in which the fluorine content is within the above-described range is effective in terms of the evenness of the thickness of the coating film or liquid saving properties and the solubility of the surfactant in the coloring composition is also good.

Examples of the fluorine-based surfactant include surfactants described in paragraph Nos. 0060 to 0064 of JP2014-041318A (paragraph Nos 0060 to 0064 of the corresponding WO2014/017669A) and the like, and surfactants described in paragraph Nos 0117 to 0132 of JP2011-132503A, the contents of which are incorporated herein by reference. Examples of a commercially available product of the fluorine-based surfactant include: MEGAFACE F171, F172, F173, F176, F177, F141, F142, F143, F144, R30, F437, F475, F479, F482, F554, F780, EXP, and MFS-330 (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC-1068, SC-381, SC-383, S-393, and KH-40 (all of which are manufactured by Asahi Glass Co., Ltd.); and POLYFOX PF636, PF656, PF6320, PF6520, and PF7002 (all of which are manufactured by OMNOVA Solutions Inc.).

In addition, as the fluorine-based surfactant, an acrylic compound, which has a molecular structure having a functional group containing a fluorine atom and in which, by applying heat to the molecular structure, the functional group containing a fluorine atom is broken to volatilize a fluorine atom, can also be suitably used. Examples of such a fluorine-based surfactant include MEGAFACE DS series manufactured by DIC Corporation (The Chemical Daily (Feb. 22, 2016) and Nikkei Business Daily (Feb. 23, 2016)) such as MEGAFACE DS-21.

In addition, it is also preferable that a polymer of a fluorine atom-containing vinyl ether compound having a fluorinated alkyl group or a fluorinated alkylene ether group, and a hydrophilic vinyl ether compound is used as the fluorine-based surfactant. Examples of such a fluorine-based surfactant include fluorine-based surfactants described in JP2016-216602A, the contents of which are incorporated herein by reference.

A block polymer can also be used as the fluorine-based surfactant. Examples thereof include the compounds described in JP2011-089090A. As the fluorine-based surfactant, a fluorine-containing polymer compound including a repeating unit derived from a (meth)acrylate compound having a fluorine atom and a repeating unit derived from a (meth)acrylate compound having 2 or more (preferably 5 or more) alkyleneoxy groups (preferably ethyleneoxy groups or propyleneoxy groups) can also be preferably used. The following compounds are also exemplified as the fluorine-based surfactant used in the present invention.

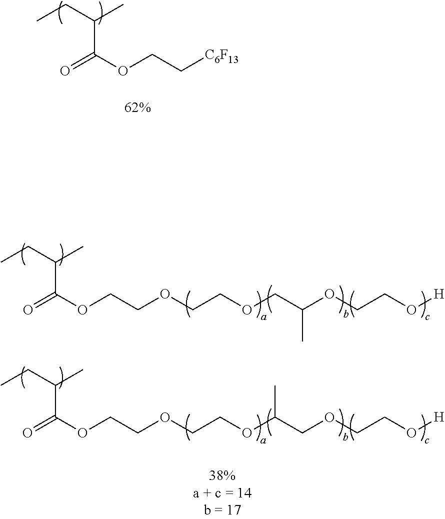

The weight-average molecular weight of the compound is preferably 3000 to 50000 and, for example, 14000. In the compound, “%” representing the proportion of a repeating unit is mol %.

In addition, as the fluorine-based surfactant, a fluorine-containing polymer having an ethylenically unsaturated bond-containing group in the side chain can be used. Specific examples thereof include compounds described in paragraph Nos. 0050 to 0090 and paragraph Nos. 0289 to 0295 of JP2010-164965A, and MEGAFACE RS-101, RS-102, RS-718K, and RS-72-K manufactured by DIC Corporation. In addition, as the fluorine-based surfactant, compounds described in paragraph Nos. 0015 to 0158 of JP2015-117327A can also be used.

Examples of the nonionic surfactant include glycerol, trimethylolpropane, trimethylolethane, an ethoxylate and propoxylate thereof (for example, glycerol propoxylate or glycerol ethoxylate), polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, sorbitan fatty acid esters, PLURONIC L10, L31, L61, L62, 10R5, 17R2, and 25R2 (manufactured by BASF), TETRONIC 304, 701, 704, 901, 904, and 150R1 (manufactured by BASF), SOLSPERSE 20000 (manufactured by Lubrizol Corporation), NCW-101, NCW-1001, and NCW-1002 (all of which are manufactured by FUJIFILM Wako Pure Chemical Corporation), PIONIN D-6112, D-6112-W, and D-6315 (all of which are manufactured by Takemoto Oil&Fat Co., Ltd.), and OLFINE E1010 and SURFYNOL 104, 400, and 440 (all of which are manufactured by Nissin Chemical Co., Ltd.).

Examples of the silicon-based surfactant include TORAY SILICONE DC3PA, TORAY SILICONE SH7PA, TORAY SILICONE DC11PA, TORAY SILICONE SH21PA, TORAY SILICONE SH28PA, TORAY SILICONE SH29PA, TORAY SILICONE SH3OPA, and TORAY SILICONE SH8400 (all of which are manufactured by Dow Corning Toray Co., Ltd.), TSF-4440, TSF-4300, TSF-4445, TSF-4460, and TSF-4452 (all of which are manufactured by Momentive Performance Materials Co., Ltd.), KP-341, KF-6001, and KF-6002 (all of which are manufactured by Shin-Etsu Chemical Co., Ltd.), and BYK307, BYK323, and BYK330 (all of which are manufactured by BYK Chemie).

The content of the surfactant in the total solid content of the coloring composition is preferably 0.001% by mass to 5.0% by mass and more preferably 0.005% to 3.0% by mass. The surfactant may be used singly or in combination of two or more kinds thereof. In a case of using two or more kinds thereof, the total amount thereof is preferably within the above-described range.

<<Ultraviolet Absorber>>

The coloring composition according to the embodiment of the present invention can contain an ultraviolet absorber. As the ultraviolet absorber, a conjugated diene compound, an aminodiene compound, a salicylate compound, a benzophenone compound, a benzotriazole compound, an acrylonitrile compound, a hydroxyphenyltriazine compound, an indole compound, a triazine compound, or the like can be used. Examples of such a compound include compounds described in paragraph Nos. 0038 to 0052 of JP2009-217221A, paragraph Nos. 0052 to 0072 of JP2012-208374A, paragraph Nos. 0317 to 0334 of JP2013-068814A, and paragraph Nos. 0061 to 0080 of JP2016-162946A, the contents of which are incorporated herein by reference. Specific examples of the ultraviolet absorber include a compound having the following structures. Examples of a commercially available product of the ultraviolet absorber include UV-503 (manufactured by Daito Chemical Co., Ltd). In addition, examples of the benzotriazole compound include MYUA series manufactured by Miyoshi Oil & Fat Co., Ltd. (The Chemical Daily, Feb. 1, 2016). In addition, as the ultraviolet absorber, compounds described in paragraph Nos. 0049 to 0059 of JP6268967B can also be used.

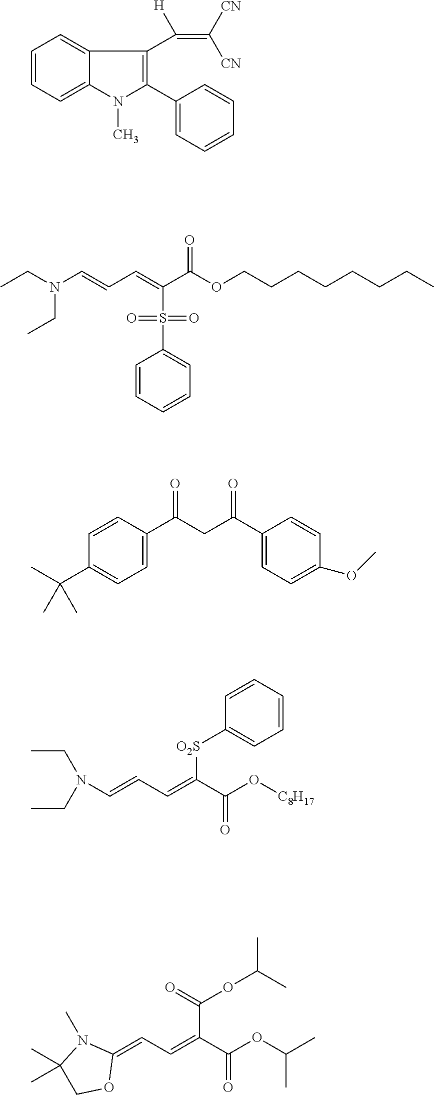

The content of the ultraviolet absorber in the total solid content of the coloring composition is preferably 0.01% to 10% by mass and more preferably 0.01% to 5% by mass. In the present invention, the ultraviolet absorber may be used singly or in combination of two or more kinds thereof. In a case where two or more kinds thereof are used, it is preferable that the total amount thereof is within the above-described range.

<<Antioxidant>>

The coloring composition according to the embodiment of the present invention can contain an antioxidant. Examples of the antioxidant include a phenol compound, a phosphite ester compound, and a thioether compound. As the phenol compound, any phenol compound which is known as a phenol-based antioxidant can be used. Preferred examples of the phenol compound include a hindered phenol compound. A compound having a substituent at a site (ortho position) adjacent to a phenolic hydroxy group is preferable. As the substituent, a substituted or unsubstituted alkyl group having 1 to 22 carbon atoms is preferable. In addition, as the antioxidant, a compound having a phenol group and a phosphite ester group in the same molecule is also preferable. In addition, as the antioxidant, a phosphorus antioxidant can also be suitably used. Examples of the phosphorus antioxidant include tris[2-[[2,4,8,10-tetrakis(1,1-dimethylethyl)dibenzo[d,f][1,3,2]dioxaphosphepin-6-yl]oxy]ethyl]amine, tris [2-[(4,6,9,11-tetra-tert-butyldibenzo[d,f][1,3,2]dioxaphosphepin-2-yl)oxy]ethyl]amine, and ethyl bis(2,4-di-tert-butyl-6-methylphenyl)phosphite. Examples of a commercially available product of the antioxidant include ADK STAB AO-20, ADK STAB AO-30, ADK STAB AO-40, ADK STAB AO-50, ADK STAB AO-50F, ADK STAB AO-60, ADK STAB AO-60G, ADK STAB AO-80, and ADK STAB AO-330 (all of which are manufactured by ADEKA Corporation). In addition, as the antioxidant, compounds described in paragraph Nos. 0023 to 0048 of JP6268967B, compounds described in WO2017/006600A, or compounds described in WO2017/164024A can also be used.

The content of the antioxidant in the total solid content of the coloring composition is preferably 0.01% to 20% by mass and more preferably 0.3% to 15% by mass. The antioxidant may be used singly or in combination of two or more kinds thereof. In a case where two or more kinds thereof are used, it is preferable that the total amount thereof is within the above-described range.

<<Other Components>>

Optionally, the coloring composition according to the embodiment of the present invention may further contain a sensitizer, a curing accelerator, a filler, a thermal curing accelerator, a plasticizer, and other auxiliary agents (for example, conductive particles, an antifoaming agent, a flame retardant, a leveling agent, a peeling accelerator, an aromatic chemical, a surface tension adjuster, or a chain transfer agent). By appropriately containing these components, properties such as film properties can be adjusted. The details of the components can be found in, for example, paragraph No. 0183 of JP2012-003225A (corresponding to paragraph No. 0237 of US2013/0034812A) and paragraph Nos. 0101 to 0104 and 0107 to 0109 of JP2008-250074A, the contents of which are incorporated herein by reference. In addition, optionally, the coloring composition according to the embodiment of the present invention may contain a potential antioxidant. Examples of the potential antioxidant include a compound in which a site functioning as an antioxidant is protected by a protecting group, and the protecting group is eliminated by heating the compound at 100° C. to 250° C. or heating the compound at 80° C. to 200° C. in the presence of an acid or basic catalyst so that the compound functions as an antioxidant. Examples of the potential antioxidant include compounds described in WO2014/021023A, WO2017/030005A, and JP2017-008219A. Examples of a commercially available product of the potential antioxidant include ADEKA ARKLS GPA-5001 (manufactured by ADEKA Corporation). In addition, as described in JP2018-155881A, C. I. Pigment Yellow 129 may be added for the purpose of improving weather fastness.

In addition, a compound represented by Formula (Q1) may be added. Specific examples thereof include 7,7,8,8,-tetracyanoquinodimethane.

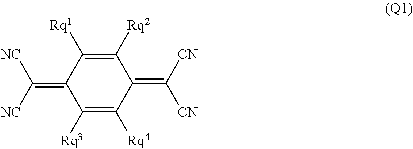

In Formula (Q1), Rq¹ to Rq⁴ each independently represent a hydrogen atom, a hydrocarbon group, a heterocyclic group, a halogen atom, a hydroxy group, an alkoxy group, an aryloxy group, an aldehyde group, an alkylcarbonyl group, an arylcarbonyl group, a carboxyl group, an alkoxycarbonyl group, an aryloxycarbonyl group, a thiol group, an alkylthio group, an arylthio group, a nitro group, an amino group, a sulfo group, a cyano group, a silyl group, a boronyl group, or a phosphino group, and Rq¹ and Rq², or Rq³ and Rq⁴ may be bonded to each other to form a ring.

The coloring composition according to the embodiment of the present invention can contain an ionic compound which is represented by Formula (1) and in which the maximum value of a molar absorption coefficient c in the visible light range is 0 to 3000.

X⁺Y⁻ (1)

In Formula (1), X⁺ is an organic or inorganic cation, and Y⁻ represents an anion having a cyano group, an anion having a nitro group, an anion having a halogenated hydrocarbon group, PF6⁻, or BF4⁻.

Specific examples of the ionic compound include potassium bis(trifluoromethanesulfonyl)imide, lithium bis(trifluoromethanesulfonyl)imide, potassium N,N-bis(nonafluorobutanesulfonyl)imide, cesium tris(trifluoromethanesulfonyl)methide, and tetrakis(pentafluofluorophenyl) lithium borate. In addition, specific examples of the ionic compound include compounds described in paragraph Nos. 0086 to 0122 of JP2016-133604A, the contents of which are incorporated herein by reference.

In order to adjust the refractive index of a film to be obtained, the coloring composition according to the embodiment of the present invention may contain a metal oxide. Examples of the metal oxide include TiO₂, ZrO₂, Al₂O₃, and SiO₂. The primary particle diameter of the metal oxide is preferably 1 to 100 nm, more preferably 3 to 70 nm, and still more preferably 5 to 50 nm. The metal oxide may have a core-shell structure. In addition, in this case, the core portion may be hollow.

The coloring composition according to the embodiment of the present invention may include a light-resistance improver. Examples of the light-resistance improver include the compounds described in paragraph Nos. 0036 and 0037 of JP2017-198787A, the compounds described in paragraph Nos 0029 to 0034 of JP2017-146350A, the compounds described in paragraph Nos. 0036 and 0037, and 0049 to 0052 of JP2017-129774A, the compounds described in paragraph Nos. 0031 to 0034 and 0058 and 0059 of JP2017-129674A, the compounds described in paragraph Nos. 0036 and 0037, and 0051 to 0054 of JP2017-122803A, the compounds described in paragraph Nos. 0025 to 0039 of WO2017/164127A, the compounds described in paragraph Nos. 0034 to 0047 of JP2017-186546A, the compounds described in paragraph Nos. 0019 to 0041 of JP2015-025116A, the compounds described in paragraph Nos. 0101 to 0125 of JP2012-145604A, the compounds described in paragraph Nos. 0018 to 0021 of JP2012-103475A, the compounds described in paragraph Nos. 0015 to 0018 of JP2011-257591A, the compounds described in paragraph Nos. 0017 to 0021 of JP2011-191483A, the compounds described in paragraph Nos. 0108 to 0116 of JP2011-145668A, and the compounds described in paragraph Nos. 0103 to 0153 of JP2011-253174A.

In the coloring composition according to the embodiment of the present invention, the content of liberated metal which is not bonded to or coordinated with a pigment or the like is preferably 100 ppm or less, more preferably 50 ppm or less, and still more preferably 10 ppm or less, it is particularly preferable to not contain the liberated metal substantially. According to this aspect, effects such as stabilization of pigment dispersibility (restraint of aggregation), improvement of spectral characteristics due to improved dispersibility, restraint of conductivity fluctuation due to stabilization of curable components or elution of metal atoms and metal ions, and improvement of display characteristics can be expected. In addition, the effects described in JP2012-153796A, JP2000-345085A, JP2005-200560A, JP1996-043620A (JP-H08-043620A), JP2004-145078A, JP2014-119487A, JP2010-083997A, JP2017-090930A, JP2018-025612A, JP2018-025797A, JP2017-155228A, JP2018-036521A, and the like can also be obtained. Examples of the types of the above-described liberated metals include Na, K, Ca, Sc, Ti, Mn, Cu, Zn, Fe, Cr, Co, Mg, Al, Sn, Zr, Ga, Ge, Ag, Au, Pt, Cs, Ni, Cd, Pb, and Bi. In addition, in the coloring composition according to the embodiment of the present invention, the content of liberated halogen which is not bonded to or coordinated with a pigment or the like is preferably 100 ppm or less, more preferably 50 ppm or less, and still more preferably 10 ppm or less, it is particularly preferable to not contain the liberated halogen substantially. Examples of halogen include F, Cl, Br, I, and anions thereof. Examples of a method for reducing liberated metals and halogens in the coloring composition include washing with ion exchange water, filtration, ultrafiltration, and purification with an ion exchange resin.

It is also preferable that the coloring composition according to the embodiment of the present invention does not substantially include terephthalic acid ester. Here, the “does not substantially include” means that the content of terephthalic acid ester is 1000 mass ppb or less in the total amount of the coloring composition, and it is more preferable to be 100 mass ppb or less and particularly preferable to be 0.

<Storage Container>

A storage container of the coloring composition according to the embodiment of the present invention is not particularly limited, and a known storage container can be used. In addition, as the storage container, in order to suppress infiltration of impurities into the raw materials or the coloring composition, a multilayer bottle in which a container interior wall having a six-layer structure is formed of six kinds of resins or a bottle in which a container interior wall having a seven-layer structure is formed of six kinds of resins is preferably used. Examples of such a container include the containers described in JP2015-123351A. In addition, for the purpose of preventing metal elution from the container interior wall, improving storage stability of the composition, and suppressing the alteration of components, it is also preferable that the container interior wall is formed of glass, stainless steel, or the like.

<Method of Preparing Coloring Composition>

The coloring composition according to the embodiment of the present invention can be prepared by mixing the above-described components with each other. In the preparation of the coloring composition, all the components may be dissolved and/or dispersed at the same time in a solvent to prepare the coloring composition, or the respective components may be appropriately left in two or more solutions or dispersion liquids and mixed to prepare the coloring composition upon use (during coating), as desired.

In addition, in the preparation of the coloring composition, a process of dispersing the pigment is preferably included. In the process for dispersing the pigment, examples of a mechanical force which is used for dispersing the pigment include compression, pressing, impact, shear, and cavitation. Specific examples of these processes include a beads mill, a sand mill, a roll mill, a ball mill, a paint shaker, a microfluidizer, a high-speed impeller, a sand grinder, a flow jet mixer, high-pressure wet atomization, and ultrasonic dispersion. In addition, in the pulverization of the pigment in a sand mill (beads mill), it is preferable to perform a treatment under the condition for increasing a pulverization efficiency by using beads having small diameters; increasing the filling rate of the beads; or the like. Incidentally, it is preferable to remove coarse particles by filtration, centrifugation, or the like after the pulverization treatment. In addition, as the process and the dispersing machine for dispersing the pigment, the process and the dispersing machine described in “Dispersion Technology Comprehension, published by Johokiko Co., Ltd., Jul. 15, 2005”, “Actual comprehensive data collection on dispersion technology and industrial application centered on suspension (solid/liquid dispersion system), published by Publication Department, Management Development Center, Oct. 10, 1978”, and paragraph No. 0022 of JP2015-157893A can be suitably used. In addition, in the process for dispersing the pigment, a refining treatment of particles in a salt milling step may be performed. With regard to the materials, equipment, treatment conditions, and the like used in the salt milling step, reference can be made to, for example, the description in JP2015-194521A and JP2012-046629A.

During the preparation of the coloring composition, it is preferable that the coloring composition is filtered through a filter, for example, in order to remove foreign matter or to reduce defects. As the filter, any filters that have been used in the related art for filtration use and the like may be used without particular limitation. Examples of a material of the filter include: a fluororesin such as polytetrafluoroethylene (PTFE); a polyamide resin such as nylon (for example, nylon-6 or nylon-6,6); and a polyolefin resin (including a polyolefin resin having a high density and an ultrahigh molecular weight) such as polyethylene or polypropylene (PP). Among these materials, polypropylene (including a high-density polypropylene) and nylon are preferable.

The pore size of the filter is preferably 0.01 to 7.0 μm, more preferably 0.01 to 3.0 μm, and still more preferably 0.05 to 0.5 μm. In a case where the pore size of the filter is within the above-described range, fine foreign matters can be reliably removed. With regard to the pore size value of the filter, reference can be made to a nominal value of filter manufacturers. As the filter, various filters provided by Nihon Pall Corporation (DFA4201NIEY and the like), Toyo Roshi Kaisha., Ltd., Nihon Entegris K.K. (formerly Nippon Microlith Co., Ltd.), Kitz Micro Filter Corporation, and the like can be used.

In addition, it is preferable that a fibrous filter material is used as the filter. Examples of the fibrous filter material include a polypropylene fiber, a nylon fiber, and a glass fiber. Examples of a commercially available product include SBP type series (SBP008 and the like), TPR type series (TPRO02, TPR005, and the like), or SHPX type series (SHPX003 and the like), all manufactured by Roki Techno Co., Ltd.

In a case of using a filter, different filters (for example, a first filter, a second filter, and the like) may be combined. In this case, the filtration with each of the filters may be performed once or may be performed twice or more times. In addition, filters having different pore sizes within the above-described range may be combined. In addition, the filtration through the first filter may be performed with only a dispersion liquid, the other components may be mixed therewith, and then the filtration through the second filter may be performed.

<Film>

The film according to the embodiment of the present invention is a film obtained from the above-described coloring composition according to the embodiment of the present invention. The film according to the embodiment of the present invention can be used for a color filter, a near-infrared transmitting filter, a near-infrared cut filter, a black matrix, a light-shielding film, a refractive index adjusting film, and the like. The film according to the embodiment of the present invention can be preferably used as a colored pixel of a color filter. Examples of the colored pixel include a red pixel, a green pixel, a blue pixel, a magenta pixel, a cyan pixel, and a yellow pixel, and a green pixel is preferable. The thickness of the film according to the embodiment of the present invention can be adjusted according to the purpose. For example, the film thickness is preferably 20 μm or less, more preferably 10 μm or less, and still more preferably 5 μm or less. The lower limit of the film thickness is preferably 0.1 gm or more, more preferably 0.2 μm or more, and still more preferably 0.3 μm or more.

<Method for Producing Film>

The film according to the embodiment of the present invention can be produced through a step of applying the above-described coloring composition according to the embodiment of the present invention on a support. The method for producing the film according to the embodiment of the present invention preferably further includes a step of forming a pattern (pixel). A photolithography method is preferable as a method of forming a pattern (pixel).

Pattern formation by a photolithography method preferably includes a step of forming a coloring composition layer on a support using the coloring composition according to the embodiment of the present invention, a step of exposing the coloring composition layer in a patterned manner, and a step of removing an non-exposed portion of the coloring composition layer by development to form a pattern (pixel). A step (pre-baking step) of baking the coloring composition layer and a step (post-baking step) of baking the developed pattern (pixel) may be provided, optionally.

<<Step of Forming Coloring Composition Layer>>

In the step of forming a coloring composition layer, the coloring composition layer is formed on a support using the coloring composition according to the embodiment of the present invention. The support is not particularly limited, and can be appropriately selected depending on applications. Examples thereof include a glass substrate and a silicon substrate, and a silicon substrate is preferable. In addition, a charge coupled device (CCD), a complementary metal-oxide semiconductor (CMOS), a transparent conductive film, or the like may be formed on the silicon substrate. In some cases, a black matrix for isolating each pixel is formed on the silicon substrate. In addition, an undercoat layer may be provided on the silicon substrate so as to improve adhesiveness to an upper layer, prevent the diffusion of materials, or planarize the surface of the substrate. The surface contact angle of the undercoat layer is preferably 20° to 70° in a case of being measured with diiodomethane. In addition, the surface contact angle of the undercoat layer is preferably 30° to 80° in a case of being measured with water. In a case where the surface contact angle of the undercoat layer is within the above-described range, coating property of the coloring composition is good. The surface contact angle of the undercoat layer can be adjusted by, for example, adding a surfactant.

As a method of applying the coloring composition, a known method can be used. Examples of the known method include: a drop casting method; a slit coating method; a spray method; a roll coating method; a spin coating method; a cast coating method; a slit and spin method; a pre-wetting method (for example, a method described in JP2009-145395A); various printing methods including jet printing such as an ink jet method (for example, an on-demand method, a piezoelectric method, or a thermal method) or a nozzle jet method, flexographic printing, screen printing, gravure printing, reverse offset printing, and metal mask printing; a transfer method using a mold or the like; and a nanoimprinting method. A method for applying the ink jet is not particularly limited, and examples thereof include a method described in “Extension of Use of Ink Jet -Infinite Possibilities in Patent-” (February, 2005, S. B. Research Co., Ltd.) (particularly pp. 115 to 133) and methods described in JP2003-262716A, JP2003-185831A, JP2003-261827A, JP2012-126830A, and JP2006-169325A. In addition, with regard to the method for applying the coloring composition, reference can be made to the description in WO2017/030174A and WO2017/018419A, the contents of which are incorporated herein by reference.

The coloring composition layer formed on the support may be dried (pre-baked). In a case of producing a film by a low-temperature process, pre-baking may not be performed. In a case of performing the pre-baking, the pre-baking temperature is preferably 150° C. or lower, more preferably 120° C. or lower, and still more preferably 110° C. or lower. The lower limit may be set to, for example, 50° C. or higher, or to 80° C. or higher. The pre-baking time is preferably 10 to 300 seconds, more preferably 40 to 250 seconds, and still more preferably 80 to 220 seconds. The pre-baking can be performed using a hot plate, an oven, or the like.

<<Exposing Step>>

Next, the coloring composition layer is exposed in a patterned manner (exposing step). For example, the coloring composition layer can be exposed in a patterned manner using a stepper exposure device or a scanner exposure device through a mask having a predetermined mask pattern. Thus, the exposed portion can be cured.

Examples of the radiation (light) which can be used during the exposure include g-rays and i-rays. In addition, light (preferably light having a wavelength of 180 to 300 nm) having a wavelength of 300 nm or less can be used. Examples of the light having a wavelength of 300 nm or less include KrF-rays (wavelength: 248 nm) and ArF-rays (wavelength: 193 nm), and KrF-rays (wavelength: 248 nm) are preferable. In addition, a long-wave light source of 300 nm or more can be used.

In addition, in a case of exposure, the composition layer may be irradiated with light continuously to expose the composition layer, or the composition layer may be irradiated with light in a pulse to expose the composition layer (pulse exposure). The pulse exposure refers to an exposing method in which light irradiation and resting are repeatedly performed in a short cycle (for example, millisecond-level or less). In a case of the pulse exposure, the pulse width is preferably 100 nanoseconds (ns) or less, more preferably 50 nanoseconds or less, and still more preferably 30 nanoseconds or less. The lower limit of the pulse width is not particularly limited, and may be 1 femtosecond (fs) or more or 10 femtoseconds or more. The frequency is preferably 1 kHz or more, more preferably 2 kHz or more, and still more preferably 4 kHz or more. The upper limit of the frequency is preferably 50 kHz or less, more preferably 20 kHz or less, and still more preferably 10 kHz or less. The maximum instantaneous illuminance is preferably 50000000 W/m² or more, more preferably 100000000 W/m² or more, and still more preferably 200000000 W/m² or more. In addition, the upper limit of the maximum instantaneous illuminance is preferably 1000000000 W/m² or less, more preferably 800000000 W/m² or less, and still more preferably 500000000 W/m² or less. The pulse width refers to a time during which light is irradiated in a pulse period. In addition, the frequency refers to the number of pulse periods per second. In addition, the maximum instantaneous illuminance refers to an average illuminance within the period of light irradiation in the pulse period. In addition, the pulse period refers to a period in which light irradiation and resting in the pulse exposure are defined as one cycle.

The irradiation amount (exposure amount) is, for example, preferably 0.03 to 2.5 J/cm² and more preferably 0.05 to 1.0 J/cm². The oxygen concentration during the exposure can be appropriately selected, and the exposure may also be performed, for example, in a low-oxygen atmosphere having an oxygen concentration of 19% by volume or less (for example, 15% by volume, 5% by volume, and substantially oxygen-free) or in a high-oxygen atmosphere having an oxygen concentration of more than 21% by volume (for example, 22% by volume, 30% by volume, and 50% by volume), in addition to an atmospheric air. In addition, the exposure illuminance can be appropriately set, and can be usually selected from a range of 1000 W/m² to 100000 W/m² (for example, 5000 W/m², 15000 W/m², or 35000 W/m²). Appropriate conditions of each of the oxygen concentration and the exposure illuminance may be combined, and for example, a combination of the oxygen concentration of 10% by volume and the illuminance of 10000 W/m², a combination of the oxygen concentration of 35% by volume and the illuminance of 20000 W/m², or the like is available.

<<Developing Step>>

Next, the non-exposed portion of the coloring composition layer is removed by development to form a pattern (pixel). The non-exposed portion of the coloring composition layer can be removed by development using a developer. Thus, the coloring composition layer of the non-exposed portion in the exposing step is eluted into the developer, and as a result, only a photocured portion remains. The temperature of the developer is preferably, for example, 20° C. to 30° C. The development time is preferably 20 to 180 seconds. In addition, in order to improve residue removing properties, a step of removing the developer by shaking off per 60 seconds and supplying a fresh developer may be repeated multiple times.

Examples of the developer include an organic solvent and an alkali developer, and an alkali developer is preferably used. As the alkali developer, an aqueous alkaline solution (alkali developer) in which an alkali agent is diluted with pure water is preferable. Examples of the alkali agent include organic alkaline compounds such as ammonia, ethylamine, diethylamine, dimethylethanolamine, diglycol amine, diethanolamine, hydroxyamine, ethylenediamine, tetramethylammonium hydroxide, tetraethylammonium hydroxide, tetrapropylammonium hydroxide, tetrabutylammonium hydroxide, ethyltrimethylammonium hydroxide, benzyltrimethylammonium hydroxide, dimethylbis(2-hydroxyethyl)ammonium hydroxide, choline, pyrrole, piperidine, and 1,8-diazabicyclo-[5.4.0]-7-undecene, and inorganic alkaline compounds such as sodium hydroxide, potassium hydroxide, sodium carbonate, sodium hydrogen carbonate, sodium silicate, and sodium metasilicate. In consideration of environmental aspects and safety aspects, the alkali agent is preferably a compound having a high molecular weight. The concentration of the alkali agent in the aqueous alkaline solution is preferably 0.001% to 10% by mass and more preferably 0.01% to 1% by mass. In addition, the developer may further contain a surfactant. Examples of the surfactant include the surfactants described above, and the surfactant is preferably a nonionic surfactant. From the viewpoint of transportation, storage, and the like, the developer may be first produced as a concentrated liquid and then diluted to a concentration required upon the use. The dilution ratio is not particularly limited, and can be set to, for example, a range of 1.5 to 100 times. In addition, it is also preferable to wash (rinse) with pure water after development. In addition, it is preferable that the rinsing is performed by supplying a rinsing liquid to the coloring composition layer after development while rotating the support on which the coloring composition layer after development is formed. In addition, it is preferable that the rinsing is performed by moving a nozzle discharging the rinsing liquid from a center of the support to a peripheral edge of the support. In this case, in the movement of the nozzle from the center of the support to the peripheral edge of the support, the nozzle may be moved while gradually decreasing the moving speed of the nozzle. By performing rinsing in this manner, in-plane variation of rinsing can be suppressed. In addition, the same effect can be obtained by gradually decreasing the rotating speed of the support while moving the nozzle from the center of the support to the peripheral edge of the support.

After the development, it is preferable to carry out an additional exposure treatment or a heating treatment (post-baking) after carrying out drying. The additional exposure treatment or the post-baking is a curing treatment after development in order to complete curing. The heating temperature in the post-baking is preferably, for example, 100° C. to 240° C. and more preferably 200° C. to 240° C. The film after development is post-baked continuously or batchwise using a heating unit such as a hot plate, a convection oven (hot air circulation dryer), and a high-frequency heater under the above-described conditions. In a case of performing the additional exposure treatment, light used for the exposure is preferably light having a wavelength of 400 nm or less. In addition, the additional exposure treatment may be carried out by the method described in KR10-2017-0122130A.

<Color Filter>

Next, the color filter according to an embodiment of the present invention will be described. The color filter according to the embodiment of the present invention has the film according to the embodiment of the present invention. Preferably, the color filter according to the embodiment of the present invention has the film according to the embodiment of the present invention as a colored pixel of the color filter. In a case where the film according to the embodiment of the present invention is used for a color filter, as the pigment, it is preferable to use a chromatic pigment. In the color filter according to the embodiment of the present invention, the thickness of the film according to the embodiment of the present invention can be appropriately adjusted depending on the purposes. The film thickness is preferably 20 μm or less, more preferably 10 μm or less, and still more preferably 5 μm or less. The lower limit of the film thickness is preferably 0.1 μm or more, more preferably 0.2 μm or more, and still more preferably 0.3 μm or more. The color filter according to the embodiment of the present invention can be used for a solid-state imaging element such as a charge coupled device (CCD) and a complementary metal-oxide semiconductor (CMOS), an image display device, or the like.

In the color filter, the width of the pixel is preferably 0.5 to 20.0 μm. The lower limit is preferably 1.0 μm or more and more preferably 2.0 μm or more. The upper limit is preferably 15.0 μm or less and more preferably 10.0 μm or less. In addition, the Young's modulus of the pixel is preferably 0.5 to 20 GPa and more preferably 2.5 to 15 GPa.

It is preferable that the pixel has high flatness. Specifically, the surface roughness Ra of the pixel is preferably 100 nm or less, more preferably 40 nm or less, and still more preferably 15 nm or less. The lower limit is not specified, but is preferably, for example, 0.1 nm or more. The surface roughness can be measured, for example, using an atomic force microscope (AFM) Dimension 3100 manufactured by Veeco Instruments, Inc. In addition, the contact angle of water on the pixel can be appropriately set to a preferred value and is typically in the range of 50° to 110°. The contact angle can be measured, for example, using a contact angle meter CV-DT-A Model (manufactured by Kyowa Interface Science Co., Ltd.). In addition, it is desired that the volume resistivity value of the pixel is high. Specifically, the volume resistivity value of the pixel is preferably 10⁹ Ω·cm or more and more preferably 10¹¹ Ω·cm or more. The upper limit is not specified, but is, for example, preferably 10¹⁴ Ω·cm or less. The volume resistivity value of the pixel can be measured, for example, using an ultra-high resistance meter 5410 (manufactured by Advantest Corporation).

In the color filter, a protective layer may be provided on the surface of the film according to the embodiment of the present invention. By providing the protective layer, various functions such as oxygen shielding, low reflection, hydrophilicity/hydrophobicity, and shielding of light (ultraviolet rays, near-infrared rays, infrared rays, and the like) having a specific wavelength can be imparted. The thickness of the protective layer is preferably 0.01 to 10 μm and still more preferably 0.1 to 5 μm. Examples of a method for forming the protective layer include a method of forming the protective layer by applying a resin composition dissolved in a solvent, a chemical vapor deposition method, and a method of attaching a molded resin with an adhesive. Examples of components constituting the protective layer include a (meth)acrylic resin, an ene-thiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamidoimide resin, a polyolefin resin, a cyclic olefin resin, a polyester resin, a styrene resin, a polyol resin, a polyvinylidene chloride resin, a melamine resin, a urethane resin, an aramid resin, a polyamide resin, an alkyd resin, an epoxy resin, a modified silicone resin, a fluororesin, a polyacrylonitrile resin, a cellulose resin, Si, C, W, Al₂O₃, Mo, SiO₂, and Si₂N₄, and two or more kinds of these components may be contained. For example, in a case of a protective layer for oxygen shielding, it is preferable that the protective layer contains a polyol resin, SiO₂, and Si₂N₄. In addition, in a case of a protective layer for low reflection, it is preferable that the protective layer contains a (meth)acrylic resin and a fluororesin.

In a case of forming the protective layer by applying a resin composition, as a method for applying the resin composition, a known method such as a spin coating method, a casting method, a screen printing method, and an ink jet method can be used. As the solvent contained in the resin composition, a known solvent (for example, propylene glycol 1-monomethyl ether 2-acetate, cyclopentanone, ethyl lactate, and the like) can be used. In a case of forming the protective layer by a chemical vapor deposition method, as the chemical vapor deposition method, a known chemical vapor deposition method (thermochemical vapor deposition method, plasma chemical vapor deposition method, and photochemical vapor deposition method) can be used.

The protective layer may contain, as desired, an additive such as organic or inorganic fine particles, an absorber of a specific wavelength (for example, ultraviolet rays, near-infrared rays, infrared rays, and the like), a refractive index adjusting agent, an antioxidant, an adhesive agent, and a surfactant. Examples of the organic or inorganic fine particles include polymer fine particles (for example, silicone resin fine particles, polystyrene fine particles, and melamine resin fine particles), titanium oxide, zinc oxide, zirconium oxide, indium oxide, aluminum oxide, titanium nitride, titanium oxynitride, magnesium fluoride, hollow silica, silica, calcium carbonate, and barium sulfate. As the absorber of a specific wavelength, a known absorber can be used. For example, as an ultraviolet absorber, a conjugated diene compound, an aminodiene compound, a salicylate compound, a benzophenone compound, a benzotriazole compound, an acrylonitrile compound, a hydroxyphenyltriazine compound, an indole compound, a triazine compound, or the like can be used. With regard to details thereof, reference can be made to the description in paragraph Nos. 0052 to 0072 of JP2012-208374A, paragraph Nos. 0317 to 0334 of JP2013-068814A, and paragraph Nos. 0061 to 0080 of JP2016-162946A, the contents of which are incorporated herein by reference. As the infrared absorber, for example, a cyclic tetrapyrrole-based coloring agent, an oxocarbon-based coloring agent, a cyanine-based coloring agent, a quaterrylene-based coloring agent, a naphthalocyanine-based coloring agent, a nickel complex-based coloring agent, a copper ion-based coloring agent, an iminium-based coloring agent, a subphthalonine-based coloring agent, a xanthene-based coloring agent, an azo-based coloring agent, a dipyrromethene-based coloring agent, a pyrrolopyrrole-based coloring agent, or the like can be used. With regard to details thereof, reference can be made to the description in paragraph Nos. 0020 to 0072 of JP2018-054760A, JP2009-263614A, and WO2017/146092A, the contents of which are incorporated herein by reference. The content of these additives can be appropriately adjusted, but is preferably 0.1% to 70% by mass and still more preferably 1% to 60% by mass with respect to the total weight of the protective layer.

In addition, as the protective layer, the protective layers described in paragraph Nos. 0073 to 0092 of JP2017-151176A can also be used.

The color filter may have a structure in which each colored pixel is embedded in a space partitioned in, for example, a lattice form by a partition wall.

The color filter may have a base layer. The base layer can also be formed using, for example, a composition obtained by removing a colorant from the above-described coloring composition according to the embodiment of the present invention.

<Solid-State Imaging Element>

The solid-state imaging element according to an embodiment of the present invention has the film according to the embodiment of the present invention. The configuration of the solid-state imaging element according to the embodiment of the present invention is not particularly limited as long as the solid-state imaging element is configured to include the film according to the embodiment of the present invention and functions as a solid-state imaging element. Examples of the configuration include the following configurations.

The solid-state imaging element is configured to have a plurality of photodiodes constituting a light receiving area of the solid-state imaging element (a charge coupled device (CCD) image sensor, a complementary metal-oxide semiconductor (CMOS) image sensor, or the like), and a transfer electrode formed of polysilicon or the like on a substrate; have a light-shielding film having openings only over the light receiving section of the photodiodes on the photodiodes and the transfer electrodes; have a device-protective film formed of silicon nitride or the like, which is formed to coat the entire surface of the light-shielding film and the light receiving section of the photodiodes, on the light-shielding film; and have a color filter on the device-protective film. Further, the solid-state imaging element may also be configured, for example, such that it has a light collecting unit (for example, a microlens, which is the same hereinafter) on a device-protective film under a color filter (a side closer to the substrate), or has a light collecting unit on a color filter. In addition, the color filter may have a structure in which each colored pixel is embedded in a space partitioned in, for example, a lattice form by a partition wall. The partition wall in this case preferably has a low refractive index for each colored pixel. Examples of an imaging apparatus having such a structure include the devices described in JP2012-227478A, JP2014-179577A, and WO2018/043654A. An imaging apparatus including the solid-state imaging element according to the embodiment of the present invention can also be used as a vehicle camera or a surveillance camera, in addition to a digital camera or electronic apparatus (mobile phones or the like) having an imaging function.

### EXAMPLES

Hereinafter, the present invention will be described in more detail with reference to the examples. The materials, the amounts of materials to be used, the proportions, the treatment details, the treatment procedure, or the like shown in the examples below may be modified appropriately as long as the modifications do not depart from the spirit of the present invention. Therefore, the scope of the present invention is not limited to the specific examples shown below.

<Preparation of Green Dispersion Liquid>

(Green Dispersion Liquid)

G pigment (green pigment), Y pigment (yellow pigment), a derivative, a dispersant, a solvent shown in the following table were mixed. Thereafter, 400 parts by mass of zirconia beads having a diameter of 0.3 mm were added thereto to perform a dispersion treatment for 5 hours using a paint shaker, and the beads were separated by filtration to produce a green dispersion liquid. The numerical values described in the following tables indicate parts by mass.

TABLE 2

| | Pigment | | | | Derivative | | Dispersant | | Solvent | |
|---|---|---|---|---|---|---|---|---|---|---|
| | G | Part by | Y | Part by | | Part by | | Part by | | Part by |
| | pigment | mass | Pigment | mass | Type | mass | Type | mass | Type | mass |
| Green dispersion liquid-1 | PG-36 | 8.73 | PY-185 | 2.18 | T-4 | 0.89 | P-1 | 12.48 | S-1 | 75.72 |
| Green dispersion liquid-2 | PG-36 | 8.73 | PY-185 | 2.18 | T-7 | 0.89 | P-1 | 12.48 | S-1 | 75.72 |
| Green dispersion liquid-3 | PG-36 | 8.73 | PY-185 | 2.18 | T-14 | 0.89 | P-1 | 12.48 | S-1 | 75.72 |
| Green dispersion liquid-4 | PG-36 | 8.73 | PY-185 | 2.18 | T-23 | 0.89 | P-1 | 12.48 | S-1 | 75.72 |
| Green dispersion liquid-5 | PG-36 | 8.73 | PY-185 | 2.18 | T-25 | 0.89 | P-1 | 12.48 | S-1 | 75.72 |
| Green dispersion liquid-6 | PG-36 | 8.73 | PY-185 | 2.18 | T-31 | 0.89 | P-1 | 12.48 | S-1 | 75.72 |
| Green dispersion liquid-7 | PG-36 | 8.73 | PY-185 | 2.18 | T-33 | 0.89 | P-1 | 12.48 | S-1 | 75.72 |
| Green dispersion liquid-8 | PG-36 | 8.73 | PY-185 | 2.18 | T-42 | 0.89 | P-1 | 12.48 | S-1 | 75.72 |
| Green dispersion liquid-9 | PG-36 | 8.73 | PY-185 | 2.18 | T-44 | 0.89 | P-1 | 12.48 | S-1 | 75.72 |
| Green dispersion liquid-10 | PG-36 | 8.73 | PY-185 | 2.18 | T-45 | 0.89 | P-1 | 12.48 | S-1 | 75.72 |
| Green dispersion liquid-11 | PG-36 | 8.73 | PY-185 | 2.18 | T-46 | 0.89 | P-1 | 12.48 | S-1 | 75.72 |
| Green dispersion liquid-12 | PG-36 | 8.73 | PY-185 | 2.18 | T-48 | 0.89 | P-1 | 12.48 | S-1 | 75.72 |
| Green dispersion liquid-13 | PG-36 | 8.73 | PY-185 | 2.18 | T-49 | 0.89 | P-1 | 12.48 | S-1 | 75.72 |
| Green dispersion liquid-14 | PG-36 | 8.73 | PY-185 | 2.18 | T-50 | 0.89 | P-1 | 12.48 | S-1 | 75.72 |
| Green dispersion liquid-15 | PG-36 | 8.73 | PY-185 | 2.18 | T-52 | 0.89 | P-1 | 12.48 | S-1 | 75.72 |
| Green dispersion liquid-16 | PG-36 | 8.73 | PY-185 | 2.18 | T-54 | 0.89 | P-1 | 12.48 | S-1 | 75.72 |
| Green dispersion liquid-17 | PG-36 | 8.73 | PY-185 | 2.18 | T-55 | 0.89 | P-1 | 12.48 | S-1 | 75.72 |
| Green dispersion liquid-18 | PG-36 | 8.73 | PY-185 | 2.18 | T-62 | 0.89 | P-1 | 12.48 | S-1 | 75.72 |
| Green dispersion liquid-19 | PG-36 | 8.73 | PY-185 | 2.18 | T-66 | 0.89 | P-1 | 12.48 | S-1 | 75.72 |
| Green dispersion liquid-20 | PG-36 | 8.73 | PY-185 | 2.18 | T-71 | 0.89 | P-1 | 12.48 | S-1 | 75.72 |
| Green dispersion liquid-21 | PG-36 | 8.73 | PY-185 | 2.18 | T-74 | 0.89 | P-1 | 12.48 | S-1 | 75.72 |
| Green dispersion liquid-22 | PG-36 | 8.73 | PY-185 | 2.18 | T-76 | 0.89 | P-11 | 12.48 | S-1 | 75.72 |
| Green dispersion liquid-23 | PG-36 | 8.73 | PY-185 | 2.18 | T-46 | 0.89 | P-2 | 12.48 | S-1 | 75.72 |
| Green dispersion liquid-24 | PG-36 | 8.73 | PY-185 | 2.18 | T-46 | 0.89 | P-3 | 12.48 | S-1 | 75.72 |
| Green dispersion liquid-25 | PG-36 | 8.73 | PY-185 | 2.18 | T-46 | 0.89 | P-4 | 12.48 | S-1 | 75.72 |
| Green dispersion liquid-26 | PG-36 | 8.73 | PY-185 | 2.18 | T-46 | 0.89 | P-5 | 12.48 | S-1 | 75.72 |
| Green dispersion liquid-27 | PG-36 | 8.73 | PY-185 | 2.18 | T-46 | 0.89 | P-6 | 12.48 | S-1 | 75.72 |
| Green dispersion liquid-28 | PG-36 | 8.73 | PY-185 | 2.18 | T-46 | 0.89 | P-7 | 12.48 | S-1 | 75.72 |
| Green dispersion liquid-29 | PG-36 | 8.73 | PY-185 | 2.18 | T-46 | 0.89 | P-8 | 12.48 | S-1 | 75.72 |
| Green dispersion liquid-30 | PG-36 | 8.73 | PY-185 | 2.18 | T-46 | 0.89 | P-9 | 12.48 | S-1 | 75.72 |
| Green dispersion liquid-31 | PG-36 | 8.73 | PY-185 | 2.18 | T-46 | 0.89 | P-10 | 12.48 | S-1 | 75.72 |
| Green dispersion liquid-32 | PG-36 | 8.73 | PY-185 | 2.18 | T-46 | 0.89 | P-12 | 12.48 | S-1 | 75.72 |
| Green dispersion liquid-33 | PG-36 | 8.73 | PY-185 | 2.18 | T-46 | 0.89 | P-13 | 12.48 | S-1 | 75.72 |
| Green dispersion liquid-34 | PG-36 | 8.73 | PY-185 | 2.18 | T-46 | 0.89 | P-14 | 12.48 | S-1 | 75.72 |
| Green dispersion liquid-35 | PG-36 | 8.73 | PY-185 | 2.18 | T-7 | 0.89 | P-10 | 12.48 | S-1 | 75.72 |
| Green dispersion liquid-36 | PG-36 | 8.73 | PY-185 | 2.18 | T-14 | 0.89 | P-10 | 12.48 | S-1 | 75.72 |
| Green dispersion liquid-37 | PG-36 | 8.73 | PY-185 | 2.18 | T-23 | 0.89 | P-10 | 12.48 | S-1 | 75.72 |
| Green dispersion liquid-38 | PG-36 | 8.73 | PY-185 | 2.18 | T-46 | 0.89 | P-10 | 12.48 | S-1 | 75.72 |
| Green dispersion liquid-39 | PG-36 | 8.73 | PY-185 | 2.18 | T-48 | 0.89 | P-10 | 12.48 | S-1 | 75.72 |
| Green dispersion liquid-40 | PG-36 | 8.73 | PY-150 | 2.18 | T-46 | 0.89 | P-1 | 12.48 | S-1 | 75.72 |
| Green dispersion liquid-41 | PG-36 | 8.73 | PY-139 | 2.18 | T-46 | 0.89 | P-1 | 12.48 | S-1 | 75.72 |
| Green dispersion liquid-42 | PG-36 | 8.73 | PY-185 | 0.50 | T-46 | 0.89 | P-1 | 12.48 | S-1 | 75.72 |
| | | | PY-150 | 1.68 | | | | | | |
| Green dispersion liquid-43 | PG-58 | 8.73 | PY-185 | 2.18 | T-46 | 0.89 | P-1 | 12.48 | S-1 | 75.72 |
| Green dispersion liquid-44 | PG-62 | 8.73 | PY-185 | 2.18 | T-46 | 0.89 | P-1 | 12.48 | S-1 | 75.72 |
| Green dispersion liquid-45 | PG-63 | 8.73 | PY-185 | 2.18 | T-46 | 0.89 | P-1 | 12.48 | S-1 | 75.72 |
| Green dispersion liquid-46 | SQ-1 | 8.73 | PY-185 | 2.18 | T-46 | 0.89 | P-1 | 12.48 | S-1 | 75.72 |
| Green dispersion liquid-47 | PG-36 | 8.71 | PY-185 | 2.17 | T-46 | 0.95 | P-1 | 12.61 | S-1 | 75.56 |
| Green dispersion liquid-48 | PG-36 | 8.61 | PY-185 | 2.18 | T-46 | 1.07 | P-1 | 13.50 | S-1 | 74.68 |
| Green dispersion liquid-49 | PG-36 | 8.42 | PY-185 | 2.1 | T-46 | 1.05 | P-1 | 15.36 | S-1 | 73.07 |
| Green dispersion liquid-r1 | PG-36 | 8.61 | PY-185 | 2.15 | Derivative-1 | 1.07 | P-1 | 13.50 | S-1 | 74.68 |
| Green dispersion liquid-r2 | PG-36 | 8.61 | PY-185 | 2.15 | Derivative-2 | 1.07 | P-1 | 13.50 | S-1 | 74.68 |

(Cyan Dispersion Liquid)

A pigment, a derivative, a dispersant, a solvent shown in the following table were mixed. Thereafter, 400 parts by mass of zirconia beads having a diameter of 0.3 mm were added thereto to perform a dispersion treatment for 5 hours using a paint shaker, and the beads were separated by filtration to produce a cyan dispersion liquid. The numerical values described in the following tables indicate parts by mass.

TABLE 3

| | Pigment | | Derivative | | Dispersant | | Solvent | |
|---|---|---|---|---|---|---|---|---|
| | | Part | | Part | | Part | | Part |
| | | by | | by | | by | | by |
| | Type | mass | Type | mass | Type | mass | Type | mass |
| Cyan | PG7/PG36/ | 10.91 | T-46 | 0.89 | P-1 | 12.48 | S-1 | 75.72 |
| dispersion | PB16 = | | | | | | | |
| liquid-1 | 1/1/1 (mass | | | | | | | |
| | ratio) | | | | | | | |

(Red Dispersion Liquid)

R pigment (red pigment), Y pigment (yellow pigment), a derivative, a dispersant, a solvent shown in the following table were mixed. Thereafter, 400 parts by mass of zirconia beads having a diameter of 0.3 mm were added thereto to perform a dispersion treatment for 5 hours using a paint shaker, and the beads were separated by filtration to produce a red dispersion liquid. The numerical values described in the following tables indicate parts by mass.

TABLE 4

| | Pigment | | | | Derivative | | Dispersant | | Solvent | |
|---|---|---|---|---|---|---|---|---|---|---|
| | R | Part by | Y | Part by | | Part by | | Part by | | Part by |
| | pigment | mass | pigment | mass | Type | mass | Type | mass | Type | mass |
| Red dispersion | PR-254 | 9.92 | PY-139 | 0.99 | T-46 | 0.89 | P-1 | 12.48 | S-1 | 75.72 |
| liquid-1 | | | | | | | | | | |
| Red dispersion | PR-254 | 9.92 | PY-139 | 0.99 | T-46 | 0.89 | P-10 | 12.48 | S-1 | 75.72 |
| liquid-2 | | | | | | | | | | |

(Blue Dispersion Liquid)

B pigment (blue pigment), V pigment (violet pigment), a derivative, a dispersant, a solvent shown in the following table were mixed. Thereafter, 400 parts by mass of zirconia beads having a diameter of 0.3 mm were added thereto to perform a dispersion treatment for 5 hours using a paint shaker, and the beads were separated by filtration to produce a blue dispersion liquid. The numerical values described in the following tables indicate parts by mass.

TABLE 5

| | Pigment | | | | Derivative | | Dispersant | | Solvent | |
|---|---|---|---|---|---|---|---|---|---|---|
| | B | Part by | V | Part by | | Part by | | Part by | | Part by |
| | pigment | mass | pigment | mass | Type | mass | Type | mass | Type | mass |
| Blue dispersion | PB15:6 | 10.91 | — | — | T-46 | 0.89 | P-1 | 12.48 | S-1 | 75.72 |
| liquid-1 | | | | | | | | | | |
| Blue dispersion | PB15:6 | 10.91 | — | — | T-46 | 0.89 | P-10 | 12.48 | S-1 | 75.72 |
| liquid-2 | | | | | | | | | | |
| Blue dispersion | PB15:6 | 8.73 | PV23 | 2.18 | T-46 | 0.89 | P-1 | 12.48 | S-1 | 75.72 |
| liquid-3 | | | | | | | | | | |

Details of the materials indicated by the abbreviations in the above tables are as follows.

[G pigment]

PG-7: C. I. Pigment Green 7

PG-36: C. I. Pigment Green 36

PG-58: C. I. Pigment Green 58

PG-62: C. I. Pigment Green 62

PG-63: C. I. Pigment Green 63

SQ-1: compound having the following structure

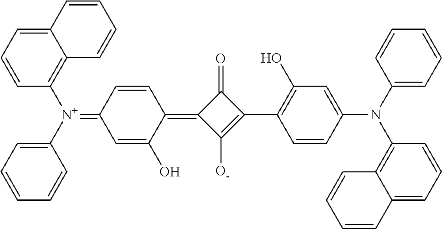

[Y pigment]

PY-139: C. I. Pigment Yellow 139

PY-150: C. I. Pigment Yellow 150

PY-185: C. I. Pigment Yellow 185

[R pigment]

PR-254: C. I. Pigment Red 254

[B pigment]

PB15:6: C. I. Pigment Blue 15:6

PB16: C. I. Pigment Blue 16

[V pigment]

PV23: C. I. Pigment Violet 23

[Derivative]

T-4, T-7, T-14, T-23, T-25, T-31, T-33, T-42, T-44, T-45, T-46, T-48, T-49, T-50, T-52, T-54, T-55, T-62, T-66, T-71, T-74, T-76: compounds having the structures described in the specific examples of the triazine compound (TA) described above

Derivative-1: compound having the following structure

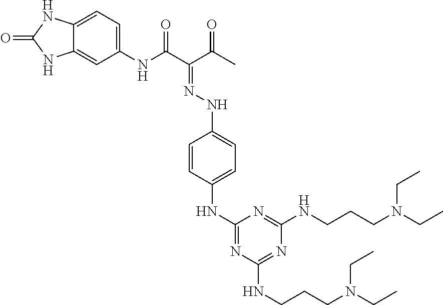 [SMILES: CCN(CC)CCCNc1nc(NCCCN(CC)CC)nc(Nc2ccc(N/N=C(\C(C)=O)C(=O)Nc3ccc4[nH]c(=O)[nH]c4c3)cc2)n1]

Derivative-2: compound having the following structure

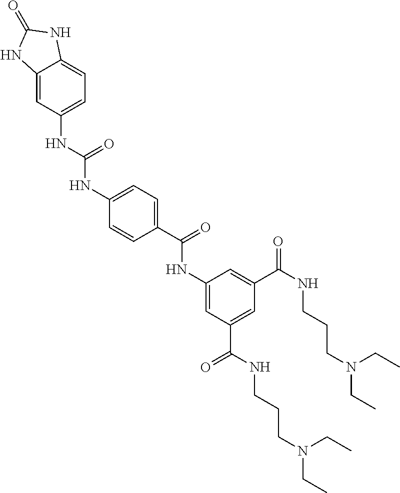 [SMILES: CCN(CC)CCCNC(=O)c1cc(NC(=O)c2ccc(NC(=O)Nc3ccc4[nH]c(=O)[nH]c4c3)cc2)cc(C(=O)NCCCN(CC)CC)c1]

In each compound used as the derivative, the maximum value (Emax) of a molar absorption coefficient in a wavelength range of 400 to 700 nm is as follows. εmax of each compound was measured as follows.

20 mg of each compound was dissolved in 200 mL of methanol, and methanol was added to 2 mL of this solution so as to be 50 mL. The absorbance of this solution was measured in a wavelength range of 200 to 800 nm using Cary 5000 UV-Vis-NIR spectrophotometer (manufactured by Agilent Technologies, Inc.) to calculate εmax. The evaluation results are listed in the following table.

A: maximum value (εmax) of a molar absorption coefficient in a wavelength range of 400 to 700 nm was 100 L·mol⁻¹·cm⁻¹ or less.

B: maximum value (εmax) of a molar absorption coefficient in a wavelength range of 400 to 700 nm was more than 100 L·mol⁻¹·cm⁻¹ and 1000 L·mol⁻¹·cm⁻¹ or less.

C: maximum value (εmax) of a molar absorption coefficient in a wavelength range of 400 to 700 nm was more than 1000 L·mol⁻¹·cm⁻¹ and 3000 L·mol⁻¹·cm⁻¹ or less.

D: maximum value (εmax) of a molar absorption coefficient in a wavelength range of 400 to 700 nm was more than 3000 L·mol⁻¹·cm⁻¹.

TABLE 6

| Type of derivative | εmax |
|---|---|
| T-4 | A |
| T-7 | A |
| T-14 | A |
| T-23 | A |
| T-25 | C |
| T-31 | A |
| T-33 | A |
| T-42 | B |
| T-44 | A |
| T-45 | A |
| T-46 | A |
| T-48 | A |
| T-49 | A |
| T-50 | A |
| T-52 | A |
| T-54 | A |
| T-55 | A |
| T-62 | A |
| T-66 | A |
| T-71 | A |
| T-74 | A |
| T-76 | A |
| Derivative-1 | D |
| Derivative-2 | A |

[Dispersant]

P-1: 30% by mass of propylene glycol monomethyl ether acetate (PGMEA) solution of a resin having the following structure (the numerical value described together with the main chain indicates a molar ratio, and the numerical value described together with the side chain indicates the number of repeating units, Mw=7000)

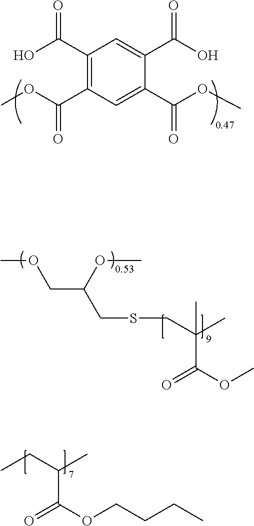

P-2: 30% by mass of PGMEA solution of a resin synthesized by the following method

50 parts by mass of methyl methacrylate, 50 parts by mass of n-butyl methacrylate, and 45.4 parts by mass of propylene glycol monomethyl ether acetate (PGMEA) were charged into a reaction container, and the atmosphere gas was replaced with nitrogen gas. The inside of the reaction container was heated to 70° C., 6 parts by mass of 3-mercapto-1,2-propanediol was added thereto, 0.12 parts by mass of azobisisobutyronitrile (AIBN) was further added thereto, and the mixture was reacted for 12 hours. It was confirmed by solid content measurement that 95% thereof was reacted. Next, 9.7 parts by mass of pyromellitic acid anhydride, 70.3 parts by mass of PGMEA, and 0.20 parts by mass of 1,8-diazabicyclo-[5.4.0]-7-undecene (DBU) as a catalyst were added thereto, and the mixture was reacted at 120° C. for 7 hours. It was confirmed by acid value measurement that 98% or more of the acid anhydride was half-esterified, and the reaction was terminated. PGMEA was added thereto to adjust non-volatile content (concentration of solid contents) to be 30% by mass, thereby obtaining a 30% by mass of PGMEA solution of a resin P-2 having an acid value of 43 mgKOH/g and a weight-average molecular weight (Mw) of 9000.

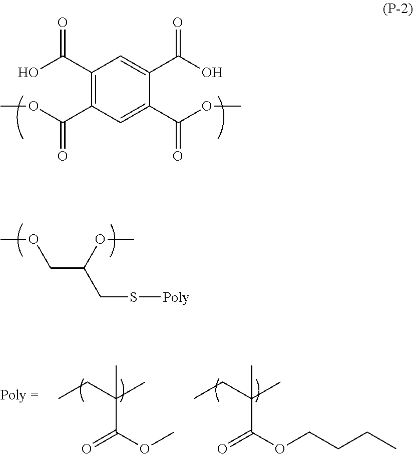

P-3: 30% by mass of PGMEA solution of a resin synthesized by the following method

50 parts by mass of methyl methacrylate, 30 parts by mass of n-butyl methacrylate, 20 parts by mass of t-butyl methacrylate, and 45.4 parts by mass of PGMEA were charged into a reaction container, and the atmosphere gas was replaced with nitrogen gas. The inside of the reaction container was heated to 70° C., 6 parts by mass of 3-mercapto-1,2-propanediol was added thereto, 0.12 parts by mass of azobisisobutyronitrile (AIBN) was further added thereto, and the mixture was reacted for 12 hours. It was confirmed by solid content measurement that 95% thereof was reacted. Next, 9.7 parts by mass of pyromellitic acid anhydride, 70.3 parts by mass of PGMEA, and 0.20 parts by mass of 1,8-diazabicyclo-[5.4.0]-7-undecene (DBU) as a catalyst were added thereto, and the mixture was reacted at 120° C. for 7 hours. It was confirmed by acid value measurement that 98% or more of the acid anhydride was half-esterified, and the reaction was terminated. PGMEA was added thereto to adjust non-volatile content (concentration of solid contents) to be 30% by mass, thereby obtaining a 30% by mass of PGMEA solution of a resin P-3 having an acid value of 43 mgKOH/g and a weight-average molecular weight (Mw) of 9000.

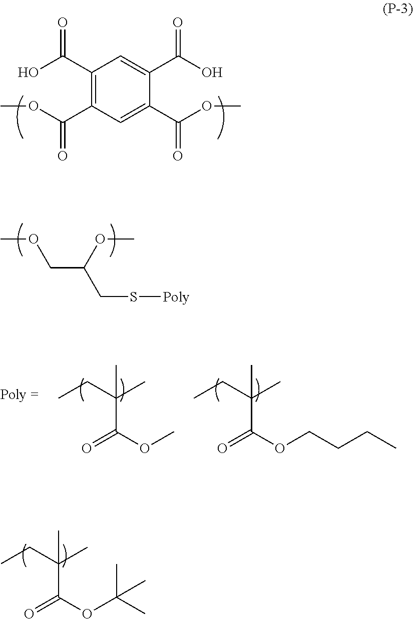

P-4: 30% by mass of PGMEA solution of a resin synthesized by the following method

A 30% by mass of PGMEA solution of a resin P-4 having an acid value of 43 mgKOH/g and a weight-average molecular weight (Mw) of 9000 was obtained in the same manner as in the synthesis of the resin P-3, except that 20 parts by mass of t-butyl methacrylate was changed to (3-ethyloxetan-3-yl)methyl methacrylate.

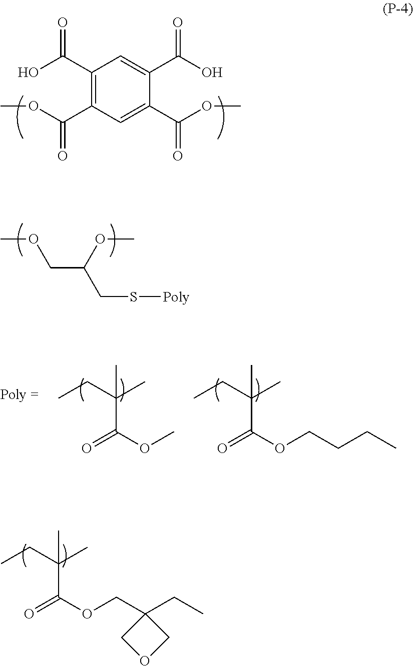

P-5: 30% by mass of PGMEA solution of a resin synthesized by the following method

A 30% by mass of PGMEA solution of a resin P-5 having an acid value of 43 mgKOH/g and a weight-average molecular weight (Mw) of 9000 was obtained in the same manner as in the synthesis of the resin P-3, except that 20 parts by mass of t-butyl methacrylate was changed to “Karenz MOI-BM” manufactured by SHOWA DENKO K.K.

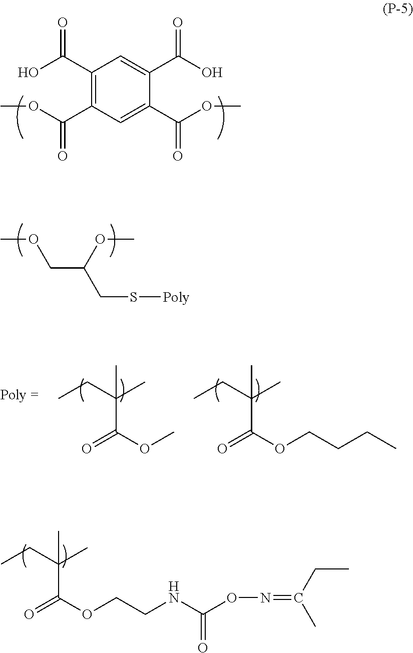

P-6: 30% by mass of PGMEA solution of a resin synthesized by the following method

6.0 parts by mass of 3-mercapto-1,2-propanediol, 9.5 parts by mass of pyromellitic acid anhydride, 62 parts by mass of PGMEA, and 0.2 parts by mass of 1,8-diazabicyclo-[5.4.0]-7-undecene were charged into a reaction container, and the atmosphere gas was replaced with nitrogen gas. The inside of the reaction container was heated to 100° C., and the mixture was reacted for 7 hours. After confirming by acid value measurement that 98% or more of the acid anhydride was half-esterified, the temperature in the system was lowered to 70° C., 53.5 parts by mass of PGMEA solution in which 65 parts by mass of methyl methacrylate, 5.0 parts by mass of ethyl acrylate, 15 parts by mass of t-butyl acrylate, 5.0 parts by mass of methacrylic acid, 10 parts by mass of hydroxyethyl methacrylate, and 0.1 parts by mass of 2,2′-azobisisobutyronitrile were dissolved was added thereto, and the mixture was reacted for 10 hours. It was confirmed by solid content measurement that the polymerization had proceeded by 95%, and the reaction was terminated. PGMEA was added thereto to adjust non-volatile content (concentration of solid contents) to be 30% by mass, thereby obtaining a 30% by mass of PGMEA solution of a resin P-6 having an acid value of 70.5 mgKOH/g and a weight-average molecular weight (Mw) of 10000.

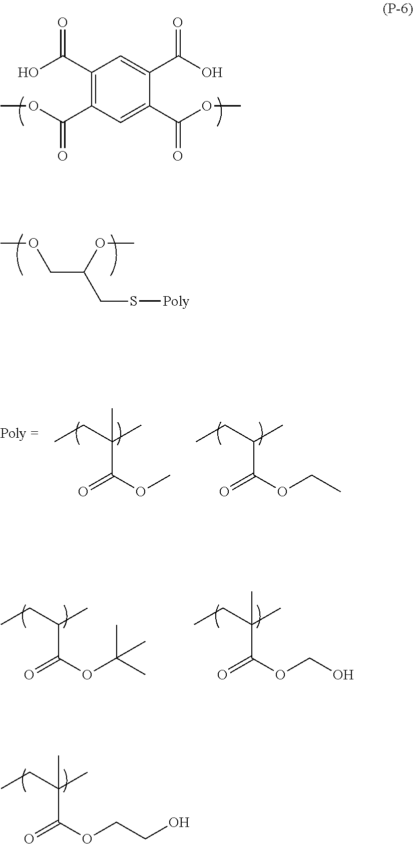

P-7: 30% by mass of PGMEA solution of a resin synthesized by the following method

108 parts by mass of 1-thioglycerol, 174 parts by mass of pyromellitic acid anhydride, 650 parts by mass of methoxypropyl acetate, and 0.2 parts by mass of monobutyltin oxide as a catalyst were charged into a reaction container, the atmosphere gas was replaced with nitrogen gas, and the mixture was reacted at 120° C. for 5 hours (first step). It was confirmed by acid value measurement that 95% or more of the acid anhydride was half-esterified. Next, 160 parts by mass of the compound obtained in the first step expressed in terms of solid contents, 200 parts by mass of 2-hydroxypropyl methacrylate, 200 parts by mass of ethyl acrylate, 150 parts by mass of t-butyl acrylate, 200 parts by mass of 2-methoxyethyl acrylate, 200 parts by mass of methyl acrylate, 50 parts by mass of methacrylic acid, and 663 parts by mass of PGMEA were charged to a reaction container, the inside of the reaction container was heated to 80° C., 1.2 parts by mass of 2,2′-azobis(2,4-dimethylvaleronitrile) was added thereto, and the mixture was reacted for 12 hours (second step). It was confirmed by solid content measurement that 95% thereof was reacted. Finally, 500 parts by mass of PGMEA solution of 50% by mass of the compound obtained in the second step, 27.0 parts by mass of 2-methacryloyloxyethyl isocyanate (MOI), 0.1 parts by mass of hydroquinone were charged to a reaction container, the reaction was performed until the disappearance of the peak of 2270 cm⁻¹ based on the isocyanate group was confirmed (third step). After confirming the disappearance of the peak, the reaction solution was cooled, and PGMEA was added thereto to adjust non-volatile content (concentration of solid contents) to be 30% by mass, thereby obtaining a 30% by mass of PGMEA solution of a resin P-7 having an acid value of 68 mgKOH/g, an unsaturated double bond value of 0.62 mmol/g, and a weight-average molecular weight (Mw) of 13000.

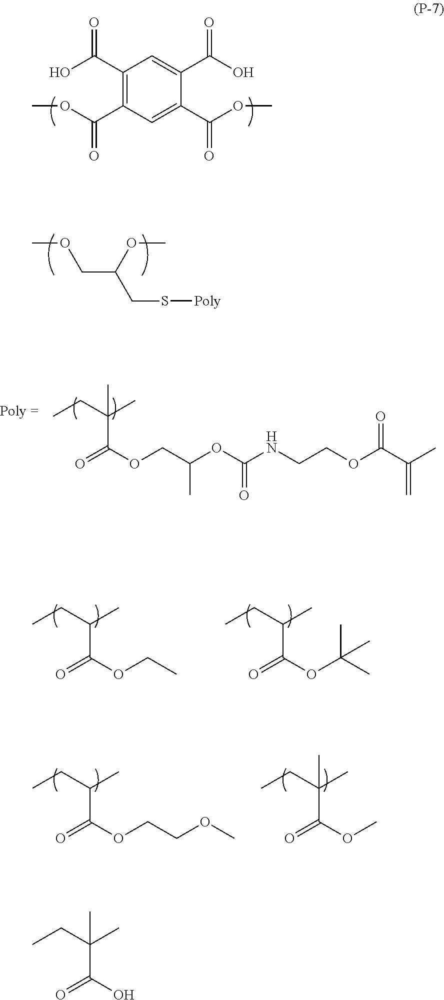

P-8: 30% by mass of propylene glycol monomethyl ether acetate (PGMEA) solution of a resin having the following structure (the numerical value described together with the main chain indicates a molar ratio, and the numerical value described together with the side chain indicates the number of repeating units, Mw=20000)

P-9: 30% by mass of PGMEA solution of a resin having the following structure (the numerical value described together with the main chain indicates a molar ratio, and the numerical value described together with the side chain indicates the number of repeating units, Mw=18000)

P-10: 30% by mass of PGMEA solution of a resin having the following structure (the numerical value described together with the main chain indicates a molar ratio, and the numerical value described together with the side chain indicates the number of repeating units, Mw=22000)

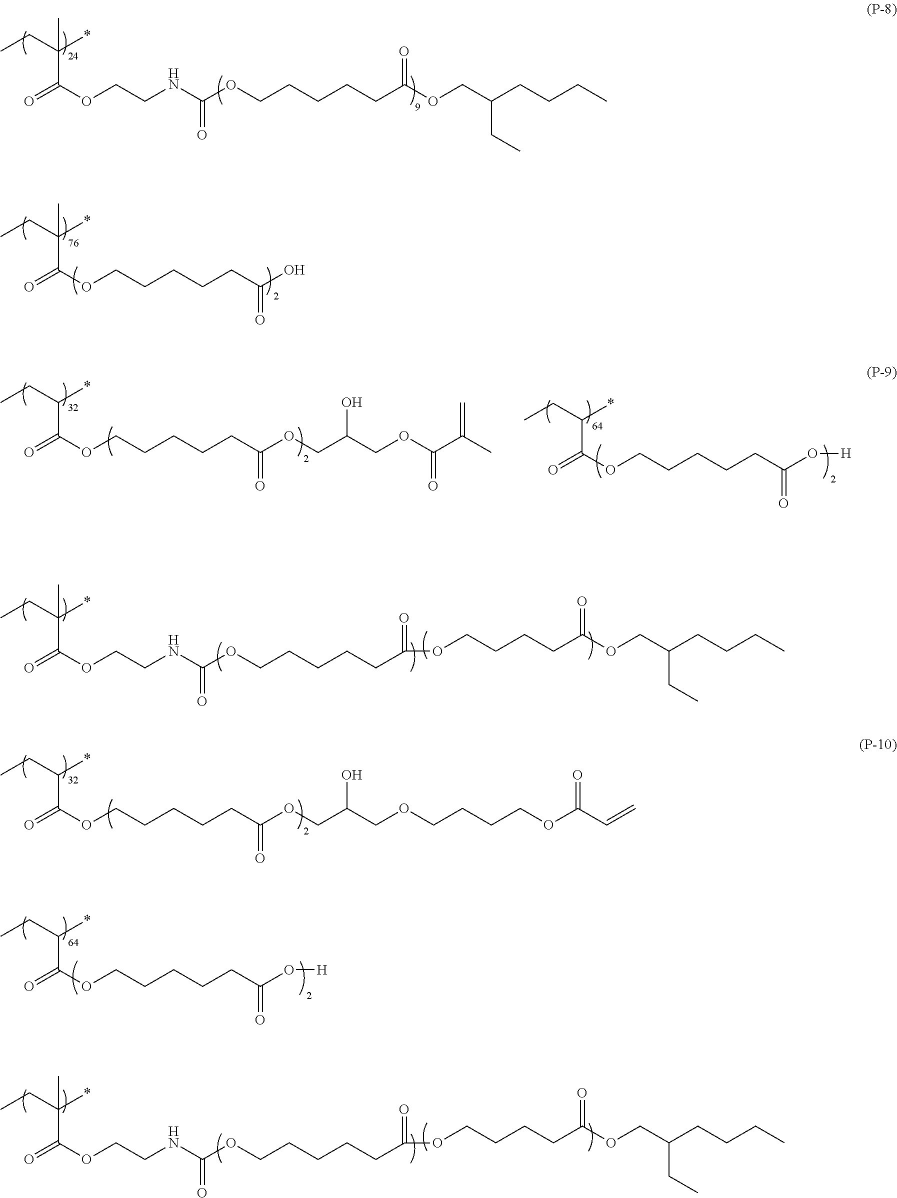

P-11: 30% by mass of PGMEA solution of a resin having the following structure (k/l/m/n=25/41/4/30 (molar ratio), p=60, q=60, Mw=22900)

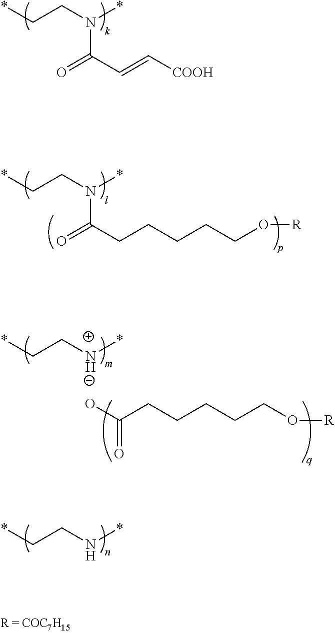

P-12: 30% by mass of PGMEA solution of a resin having the following structure (the numerical value described together with the side chain indicates the number of repeating units, Mw=18000)

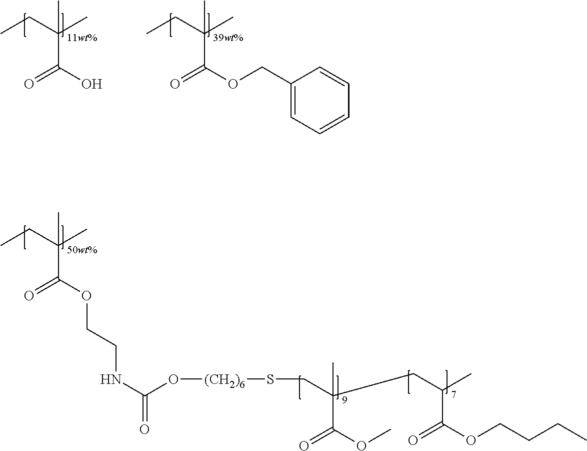

P-13: 30% by mass of PGMEA solution of a resin having the following structure (the numerical value described together with the side chain indicates the number of repeating units, Mw=18000)

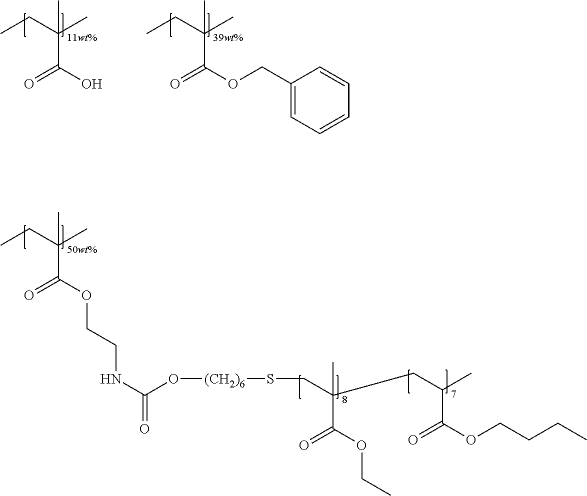

P-14: 30% by mass of PGMEA solution of a resin having the following structure (the numerical value described together with the side chain indicates the number of repeating units, Mw=18000)

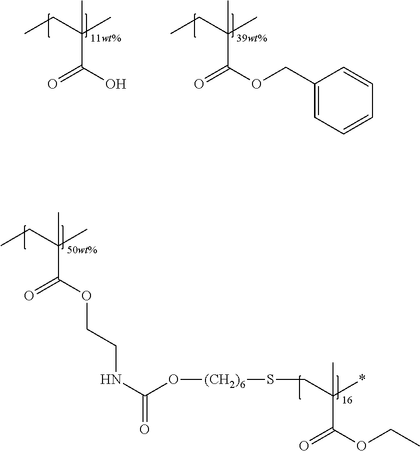

[Solvent]

S-1: propylene glycol monomethyl ether acetate (PGMEA)

<Preparation of Coloring Composition>

Raw materials having the compositions shown in the following tables were mixed to prepare a coloring composition.

TABLE 7

| | | | | | Polymerizable | | Photopolymerization | | | | Polymerization | | Solvent | |
|---|---|---|---|---|---|---|---|---|---|---|---|---|---|---|
| | Dispersion liquid | | Resin | | compound | | initiator | | Surfactant | | inhibitor | | | |
| | | Part by | | Part by | | Part by | | Part by | | Part by | | Part by | | Part by |
| | Type | mass | Type | mass | Type | mass | Type | mass | Type | mass | Type | mass | Type | mass |
| Example-1 | Green dispersion | 82.69 | D-1 | 0.38 | E-1 | 1.18 | F-1 | 0.58 | W-1 | 0.77 | G-1 | 0.0011 | S-1 | 14.40 |
| | liquid-1 | | | | | | | | | | | | | |
| Example-2 | Green dispersion | 82.69 | D-1 | 0.38 | E-1 | 1.18 | F-1 | 0.58 | W-1 | 0.77 | G-1 | 0.0011 | S-1 | 14.40 |
| | liquid-2 | | | | | | | | | | | | | |
| Example-3 | Green dispersion | 82.69 | D-1 | 0.38 | E-1 | 1.18 | F-1 | 0.58 | W-1 | 0.77 | G-1 | 0.0011 | S-1 | 14.40 |
| | liquid-3 | | | | | | | | | | | | | |
| Example-4 | Green dispersion | 82.69 | D-1 | 0.38 | E-1 | 1.18 | F-1 | 0.58 | W-1 | 0.77 | G-1 | 0.0011 | S-1 | 14.40 |
| | liquid-4 | | | | | | | | | | | | | |
| Example-5 | Green dispersion | 82.69 | D-1 | 0.38 | E-1 | 1.18 | F-1 | 0.58 | W-1 | 0.77 | G-1 | 0.0011 | S-1 | 14.40 |
| | liquid-5 | | | | | | | | | | | | | |
| Example-6 | Green dispersion | 82.69 | D-1 | 0.38 | E-1 | 1.18 | F-1 | 0.58 | W-1 | 0.77 | G-1 | 0.0011 | S-1 | 14.40 |
| | liquid-6 | | | | | | | | | | | | | |
| Example-7 | Green dispersion | 82.69 | D-1 | 0.38 | E-1 | 1.18 | F-1 | 0.58 | W-1 | 0.77 | G-1 | 0.0011 | S-1 | 14.40 |
| | liquid-7 | | | | | | | | | | | | | |
| Example-8 | Green dispersion | 82.69 | D-1 | 0.38 | E-1 | 1.18 | F-1 | 0.58 | W-1 | 0.77 | G-1 | 0.0011 | S-1 | 14.40 |
| | liquid-8 | | | | | | | | | | | | | |
| Example-9 | Green dispersion | 82.69 | D-1 | 0.38 | E-1 | 1.18 | F-1 | 0.58 | W-1 | 0.77 | G-1 | 0.0011 | S-1 | 14.40 |
| | liquid-9 | | | | | | | | | | | | | |
| Example-10 | Green dispersion | 82.69 | D-1 | 0.38 | E-1 | 1.18 | F-1 | 0.58 | W-1 | 0.77 | G-1 | 0.0011 | S-1 | 14.40 |
| | liquid-10 | | | | | | | | | | | | | |
| Example-11 | Green dispersion | 82.69 | D-1 | 0.38 | E-1 | 1.18 | F-1 | 0.58 | W-1 | 0.77 | G-1 | 0.0011 | S-1 | 14.40 |
| | liquid-11 | | | | | | | | | | | | | |
| Example-12 | Green dispersion | 82.69 | D-1 | 0.38 | E-1 | 1.18 | F-1 | 0.58 | W-1 | 0.77 | G-1 | 0.0011 | S-1 | 14.40 |
| | liquid-12 | | | | | | | | | | | | | |
| Example-13 | Green dispersion | 82.69 | D-1 | 0.38 | E-1 | 1.18 | F-1 | 0.58 | W-1 | 0.77 | G-1 | 0.0011 | S-1 | 14.40 |
| | liquid-13 | | | | | | | | | | | | | |
| Example-14 | Green dispersion | 82.69 | D-1 | 0.38 | E-1 | 1.18 | F-1 | 0.58 | W-1 | 0.77 | G-1 | 0.0011 | S-1 | 14.40 |
| | liquid-14 | | | | | | | | | | | | | |
| Example-15 | Green dispersion | 82.69 | D-1 | 0.38 | E-1 | 1.18 | F-1 | 0.58 | W-1 | 0.77 | G-1 | 0.0011 | S-1 | 14.40 |
| | liquid-15 | | | | | | | | | | | | | |
| Example-16 | Green dispersion | 82.69 | D-1 | 0.38 | E-1 | 1.18 | F-1 | 0.58 | W-1 | 0.77 | G-1 | 0.0011 | S-1 | 14.40 |
| | liquid-16 | | | | | | | | | | | | | |
| Example-17 | Green dispersion | 82.69 | D-1 | 0.38 | E-1 | 1.18 | F-1 | 0.58 | W-1 | 0.77 | G-1 | 0.0011 | S-1 | 14.40 |
| | liquid-17 | | | | | | | | | | | | | |
| Example-18 | Green dispersion | 82.69 | D-1 | 0.38 | E-1 | 1.18 | F-1 | 0.58 | W-1 | 0.77 | G-1 | 0.0011 | S-1 | 14.40 |
| | liquid-18 | | | | | | | | | | | | | |
| Example-19 | Green dispersion | 82.69 | D-1 | 0.38 | E-1 | 1.18 | F-1 | 0.58 | W-1 | 0.77 | G-1 | 0.0011 | S-1 | 14.40 |
| | liquid-19 | | | | | | | | | | | | | |
| Example-20 | Green dispersion | 82.69 | D-1 | 0.38 | E-1 | 1.18 | F-1 | 0.58 | W-1 | 0.77 | G-1 | 0.0011 | S-1 | 14.40 |
| | liquid-20 | | | | | | | | | | | | | |
| Example-21 | Green dispersion | 82.69 | D-1 | 0.38 | E-1 | 1.18 | F-1 | 0.58 | W-1 | 0.77 | G-1 | 0.0011 | S-1 | 14.40 |
| | liquid-21 | | | | | | | | | | | | | |
| Example-22 | Green dispersion | 82.69 | D-1 | 0.38 | E-1 | 1.18 | F-1 | 0.58 | W-1 | 0.77 | G-1 | 0.0011 | S-1 | 14.40 |
| | liquid-22 | | | | | | | | | | | | | |
| Example-23 | Green dispersion | 82.69 | D-1 | 0.38 | E-1 | 1.18 | F-1 | 0.58 | W-1 | 0.77 | G-1 | 0.0011 | S-1 | 14.40 |
| | liquid-23 | | | | | | | | | | | | | |
| Example-24 | Green dispersion | 82.69 | D-1 | 0.38 | E-1 | 1.18 | F-1 | 0.58 | W-1 | 0.77 | G-1 | 0.0011 | S-1 | 14.40 |
| | liquid-24 | | | | | | | | | | | | | |
| Example-25 | Green dispersion | 82.69 | D-1 | 0.38 | E-1 | 1.18 | F-1 | 0.58 | W-1 | 0.77 | G-1 | 0.0011 | S-1 | 14.40 |
| | liquid-25 | | | | | | | | | | | | | |

TABLE 8

| | | | | | Polymerizable | | Photopolymerization | | | | Polymerization | | | |
|---|---|---|---|---|---|---|---|---|---|---|---|---|---|---|
| | Dispersion liquid | | Resin | | compound | | initiator | | Surfactant | | inhibitor | | Solvent | |
| | | Part by | | Part by | | Part by | | Part by | | Part by | | Part by | | Part by |
| | Type | mass | Type | mass | Type | mass | Type | mass | Type | mass | Type | mass | Type | mass |
| Example-26 | Green dispersion | 82.69 | D-1 | 0.38 | E-1 | 1.18 | F-1 | 0.58 | W-1 | 0.77 | G-1 | 0.0011 | S-1 | 14.40 |
| | liquid-26 | | | | | | | | | | | | | |
| Example-27 | Green dispersion | 82.69 | D-1 | 0.38 | E-1 | 1.18 | F-1 | 0.58 | W-1 | 0.77 | G-1 | 0.0011 | S-1 | 14.40 |
| | liquid-27 | | | | | | | | | | | | | |
| Example-28 | Green dispersion | 82.69 | D-1 | 0.38 | E-1 | 1.18 | F-1 | 0.58 | W-1 | 0.77 | G-1 | 0.0011 | S-1 | 14.40 |
| | liquid-28 | | | | | | | | | | | | | |
| Example-29 | Green dispersion | 82.69 | D-1 | 0.38 | E-1 | 1.18 | F-1 | 0.58 | W-1 | 0.77 | G-1 | 0.0011 | S-1 | 14.40 |
| | liquid-29 | | | | | | | | | | | | | |
| Example-30 | Green dispersion | 82.69 | D-1 | 0.38 | E-1 | 1.18 | F-1 | 0.58 | W-1 | 0.77 | G-1 | 0.0011 | S-1 | 14.40 |
| | liquid-30 | | | | | | | | | | | | | |
| Example-31 | Green dispersion | 82.69 | D-1 | 0.38 | E-1 | 1.18 | F-1 | 0.58 | W-1 | 0.77 | G-1 | 0.0011 | S-1 | 14.40 |
| | liquid-31 | | | | | | | | | | | | | |
| Example-32 | Green dispersion | 82.69 | D-1 | 0.38 | E-1 | 1.18 | F-1 | 0.58 | W-1 | 0.77 | G-1 | 0.0011 | S-1 | 14.40 |
| | liquid-32 | | | | | | | | | | | | | |
| Example-33 | Green dispersion | 82.69 | D-1 | 0.38 | E-1 | 1.18 | F-1 | 0.58 | W-1 | 0.77 | G-1 | 0.0011 | S-1 | 14.40 |
| | liquid-33 | | | | | | | | | | | | | |
| Example-34 | Green dispersion | 82.69 | D-1 | 0.38 | E-1 | 1.18 | F-1 | 0.58 | W-1 | 0.77 | G-1 | 0.0011 | S-1 | 14.40 |
| | liquid-34 | | | | | | | | | | | | | |
| Example-35 | Green dispersion | 82.69 | D-1 | 0.38 | E-1 | 1.18 | F-1 | 0.58 | W-1 | 0.77 | G-1 | 0.0011 | S-1 | 14.40 |
| | liquid-35 | | | | | | | | | | | | | |
| Example-36 | Green dispersion | 82.69 | D-1 | 0.38 | E-1 | 1.18 | F-1 | 0.58 | W-1 | 0.77 | G-1 | 0.0011 | S-1 | 14.40 |
| | liquid-36 | | | | | | | | | | | | | |
| Example-37 | Green dispersion | 82.69 | D-1 | 0.38 | E-1 | 1.18 | F-1 | 0.58 | W-1 | 0.77 | G-1 | 0.0011 | S-1 | 14.40 |
| | liquid-37 | | | | | | | | | | | | | |
| Example-38 | Green dispersion | 82.69 | D-1 | 0.38 | E-1 | 1.18 | F-1 | 0.58 | W-1 | 0.77 | G-1 | 0.0011 | S-1 | 14.40 |
| | liquid-38 | | | | | | | | | | | | | |
| Example-39 | Green dispersion | 82.69 | D-1 | 0.38 | E-1 | 1.18 | F-1 | 0.58 | W-1 | 0.77 | G-1 | 0.0011 | S-1 | 14.40 |
| | liquid-39 | | | | | | | | | | | | | |
| Example-40 | Green dispersion | 82.69 | D-1 | 0.38 | E-1 | 1.18 | F-1 | 0.58 | W-1 | 0.77 | G-1 | 0.0011 | S-1 | 14.40 |
| | liquid-40 | | | | | | | | | | | | | |
| Example-41 | Green dispersion | 82.69 | D-1 | 0.38 | E-1 | 1.18 | F-1 | 0.58 | W-1 | 0.77 | G-1 | 0.0011 | S-1 | 14.40 |
| | liquid-41 | | | | | | | | | | | | | |
| Example-42 | Green dispersion | 82.69 | D-1 | 0.38 | E-1 | 1.18 | F-1 | 0.58 | W-1 | 0.77 | G-1 | 0.0011 | S-1 | 14.40 |
| | liquid-42 | | | | | | | | | | | | | |
| Example-43 | Green dispersion | 82.69 | D-1 | 0.38 | E-1 | 1.18 | F-1 | 0.58 | W-1 | 0.77 | G-1 | 0.0011 | S-1 | 14.40 |
| | liquid-43 | | | | | | | | | | | | | |
| Example-44 | Green dispersion | 82.69 | D-1 | 0.38 | E-1 | 1.18 | F-1 | 0.58 | W-1 | 0.77 | G-1 | 0.0011 | S-1 | 14.40 |
| | liquid-44 | | | | | | | | | | | | | |
| Example-45 | Green dispersion | 82.69 | D-1 | 0.38 | E-1 | 1.18 | F-1 | 0.58 | W-1 | 0.77 | G-1 | 0.0011 | S-1 | 14.40 |
| | liquid-45 | | | | | | | | | | | | | |
| Example-46 | Green dispersion | 82.69 | D-1 | 0.38 | E-1 | 1.18 | F-1 | 0.58 | W-1 | 0.77 | G-1 | 0.0011 | S-1 | 14.40 |
| | liquid-46 | | | | | | | | | | | | | |
| Example-47 | Green dispersion | 82.32 | D-1 | 0.56 | E-1 | 1.40 | F-1 | 0.62 | W-1 | 0.77 | G-1 | 0.0011 | S-1 | 14.33 |
| | liquid-47 | | | | | | | | | | | | | |
| Example-48 | Green dispersion | 82.17 | D-1 | 0.75 | E-1 | 1.40 | F-1 | 0.62 | W-1 | 0.77 | G-1 | 0.0011 | S-1 | 14.30 |
| | liquid-48 | | | | | | | | | | | | | |
| Example-49 | Green dispersion | 81.67 | D-1 | 0.85 | E-1 | 1.52 | F-1 | 0.63 | W-1 | 0.78 | G-1 | 0.0011 | S-1 | 14.55 |
| | liquid-49 | | | | | | | | | | | | | |
| Example-50 | Green dispersion | 82.69 | D-1 | 0.38 | E-2 | 1.18 | F-1 | 0.58 | W-1 | 0.77 | G-1 | 0.0011 | S-1 | 14.40 |
| | liquid-11 | | | | | | | | | | | | | |

TABLE 9

| | | | | | Poly- | | Photo- | | | | Poly- | | | |
|---|---|---|---|---|---|---|---|---|---|---|---|---|---|---|
| | Dispersion | | | | merizable | | polymerization | | | | merization | | | |
| | liquid | | Resin | | compound | | initiator | | Surfactant | | inhibitor | | Solvent | |
| | | Part | | Part | | Part | | Part | | Part | | Part | | Part |
| | | by | | by | | by | | by | | by | | by | | by |
| | Type | mass | Type | mass | Type | mass | Type | mass | Type | mass | Type | mass | Type | mass |
| Example-51 | Green dispersion liquid-11 | 82.69 | D-1 | 0.38 | E-3 | 1.18 | F-1 | 0.58 | W-1 | 0.77 | G-1 | 0.0011 | S-1 | 14.40 |
| Example-52 | Green dispersion liquid-11 | 82.69 | D-1 | 0.38 | E-4 | 1.18 | F-1 | 0.58 | W-1 | 0.77 | G-1 | 0.0011 | S-1 | 14.40 |
| Example-53 | Green dispersion liquid-11 | 82.69 | D-1 | 0.38 | E-5 | 1.18 | F-1 | 0.58 | W-1 | 0.77 | G-1 | 0.0011 | S-1 | 14.40 |
| Example-54 | Green dispersion liquid-11 | 82.69 | D-1 | 0.38 | E-1 | 0.59 | F-1 | 0.58 | W-1 | 0.77 | G-1 | 0.0011 | S-1 | 14.40 |
| | | | | | E-2 | 0.59 | | | | | | | | |
| Example-55 | Green dispersion liquid-11 | 82.69 | D-1 | 0.38 | E-1 | 1.18 | F-2 | 0.58 | W-1 | 0.77 | G-1 | 0.0011 | S-1 | 14.40 |
| Example-56 | Green dispersion liquid-11 | 82.69 | D-1 | 0.38 | E-1 | 1.18 | F-3 | 0.58 | W-1 | 0.77 | G-1 | 0.0011 | S-1 | 14.40 |
| Example-57 | Green dispersion liquid-11 | 82.69 | D-1 | 0.38 | E-1 | 1.18 | F-4 | 0.58 | W-1 | 0.77 | G-1 | 0.0011 | S-1 | 14.40 |
| Example-58 | Green dispersion liquid-11 | 82.69 | D-1 | 0.38 | E-1 | 1.18 | F-5 | 0.58 | W-1 | 0.77 | G-1 | 0.0011 | S-1 | 14.40 |
| Example-59 | Green dispersion liquid-11 | 82.69 | D-1 | 0.38 | E-1 | 1.18 | F-1 | 0.29 | W-1 | 0.77 | G-1 | 0.0011 | S-1 | 14.40 |
| | | | | | | | F-2 | 0.29 | | | | | | |
| Example-60 | Green dispersion liquid-48 | 82.17 | D-1 | 0.75 | E-1 | 1.40 | F-4 | 0.62 | W-1 | 0.77 | G-1 | 0.0011 | S-1 | 14.30 |
| Example-61 | Green dispersion liquid-4 | 82.69 | D-1 | 0.38 | E-1 | 1.18 | F-1 | 0.58 | W-1 | 0.77 | G-1 | 0.0011 | S-1 | 14.40 |
| | Green dispersion liquid-11 | | | | | | | | | | | | | |
| Example-62 | Green dispersion liquid-11 | 82.69 | D-2 | 0.38 | E-1 | 1.18 | F-1 | 0.58 | W-1 | 0.77 | G-1 | 0.0011 | S-1 | 14.40 |
| Example-63 | Green dispersion liquid-11 | 82.69 | D-1 | 0.19 | E-1 | 1.18 | F-1 | 0.58 | W-1 | 0.77 | G-1 | 0.0011 | S-1 | 14.40 |
| | | | D-2 | 0.19 | | | | | | | | | | |
| Example-64 | Green dispersion liquid-11 | 82.69 | D-1 | 0.38 | E-1 | 1.18 | F-1 | 0.58 | W-1 | 0.77 | G-1 | 0.0011 | S-2 | 14.40 |
| Example-65 | Green dispersion liquid-11 | 82.69 | D-1 | 0.38 | E-1 | 1.18 | F-1 | 0.58 | W-1 | 0.77 | G-1 | 0.0011 | S-1 | 14.40 |
| | Green dispersion liquid-28 | | | | | | | | | | | | | |
| Example-66 | Green dispersion liquid-11 | 74.698.00 | D-1 | 0.38 | E-1 | 1.18 | F-1 | 0.58 | W-1 | 0.77 | G-1 | 0.0011 | S-1 | 14.40 |
| | Green dispersion liquid-r1 | | | | | | | | | | | | | |
| Example-67 | Cyan dispersion liquid-1 | 82.69 | D-1 | 0.38 | E-1 | 1.18 | F-2 | 0.58 | W-1 | 0.77 | G-1 | 0.0011 | S-1 | 14.40 |
| Comparative | Green dispersion liquid-r1 | 82.17 | D-1 | 0.75 | E-1 | 1.40 | F-1 | 0.62 | W-1 | 0.77 | G-1 | 0.0011 | S-1 | 14.30 |
| example-1 | | | | | | | | | | | | | | |
| Comparative | Green dispersion liquid-r2 | 82.17 | D-1 | 0.75 | E-1 | 1.40 | F-4 | 0.62 | W-1 | 0.77 | G-1 | 0.0011 | S-1 | 14.30 |
| example-2 | | | | | | | | | | | | | | |

TABLE 10

| | | | | | Poly- | | Photo- | | | | Poly- | | | |
|---|---|---|---|---|---|---|---|---|---|---|---|---|---|---|
| | Dispersion | | | | merizable | | polymerization | | | | merization | | | |
| | liquid | | Resin | | compound | | initiator | | Surfactant | | inhibitor | | Solvent | |
| | | Part | | Part | | Part | | Part | | Part | | Part | | Part |
| | | by | | by | | by | | by | | by | | by | | by |
| | Type | mass | Type | mass | Type | mass | Type | mass | Type | mass | Type | mass | Type | mass |
| Example-101 | Red dispersion liquid-1 | 82.69 | D-1 | 0.38 | E-1 | 1.18 | F-1 | 0.58 | W-1 | 0.77 | G-1 | 0.0011 | S-1 | 14.40 |
| Example-102 | Red dispersion liquid-2 | 82.69 | D-1 | 0.38 | E-1 | 1.18 | F-1 | 0.58 | W-1 | 0.77 | G-1 | 0.0011 | S-1 | 14.40 |

TABLE 11

| | | | | | Poly- | | Photo- | | | | Poly- | | | |
|---|---|---|---|---|---|---|---|---|---|---|---|---|---|---|
| | | | | | merizable | | polymerization | | | | merization | | | |
| | Dispersion liquid | | Resin | | compound | | initiator | | Surfactant | | inhibitor | | Solvent | |
| | | Part | | Part | | Part | | Part | | Part | | Part | | Part |
| | | by | | by | | by | | by | | by | | by | | by |
| | Type | mass | Type | mass | Type | mass | Type | mass | Type | mass | Type | mass | Type | mass |
| Example-201 | Blue dispersion liquid-1 | 82.69 | D-1 | 0.38 | E-1 | 1.18 | F-1 | 0.58 | W-1 | 0.77 | G-1 | 0.0011 | S-1 | 14.40 |
| Example-202 | Blue dispersion liquid-2 | 82.69 | D-1 | 0.38 | E-1 | 1.18 | F-1 | 0.58 | W-1 | 0.77 | G-1 | 0.0011 | S-1 | 14.40 |

TABLE 12

| | | | | | | | Poly- | | Photopoly- | | | | Poly- | | | | | |
|---|---|---|---|---|---|---|---|---|---|---|---|---|---|---|---|---|---|---|
| | Dispersion | | | | | | merizable | | merization | | | | merization | | | | | |
| | liquid | | Dye | | Resin | | compound | | initiator | | Surfactant | | inhibitor | | Additive | | Solvent | |
| | | Part | | Part | | Part | | Part | | Part | | Part | | Part | | Part | | Part |
| | | by | | by | | by | | by | | by | | by | | by | | by | | by |
| | Type | mass | Type | mass | Type | mass | Type | mass | Type | mass | Type | mass | Type | mass | Type | mass | Type | mass |
| Exam- | Blue | 68.91 | Dye 1 | 1.50 | D-1 | 0.57 | E-1 | 1.18 | F-1 | 0.58 | W-1 | 0.77 | G-1 | 0.0011 | Additive | 0.45 | S-1 | 14.40 |
| ple- | dispersion | | | | | | | | | | | | | | 1 | | S-2 | 24.44 |
| 203 | liquid-3 | | | | | | | | | | | | | | | | | |
| Exam- | Blue | 68.91 | Dye 2 | 1.50 | D-1 | 0.57 | E-1 | 1.18 | F-1 | 0.58 | W-1 | 0.77 | G-1 | 0.0011 | Additive | 0.45 | S-1 | 14.40 |
| ple- | dispersion | | | | | | | | | | | | | | 1 | | S-2 | 24.44 |
| 204 | liquid-3 | | | | | | | | | | | | | | | | | |
| Exam- | Blue | 68.91 | Dye 3 | 1.50 | D-1 | 0.57 | E-1 | 1.18 | F-1 | 0.58 | W-1 | 0.77 | G-1 | 0.0011 | Additive | 0.45 | S-1 | 14.40 |
| ple- | dispersion | | | | | | | | | | | | | | 1 | | S-2 | 24.44 |
| 205 | liquid-3 | | | | | | | | | | | | | | | | | |

Details of the materials indicated by the abbreviations in the above tables are as follows.

(Dispersion Liquid)

Green dispersion liquid-1 to green dispersion liquid-49, green dispersion liquid-r1, green dispersion liquid-r2, cyan dispersion liquid-1, red dispersion liquid-1, red dispersion liquid-2, blue dispersion liquid-1, blue dispersion liquid-2, blue dispersion liquid-3: dispersion liquids described above

(Resin)

D-1: resin having the following structure (the numerical value described together with the main chain indicates a molar ratio, Mw=11000)

D-2: resin having the following structure (the numerical value described together with the main chain indicates a molar ratio, Mw=14000)

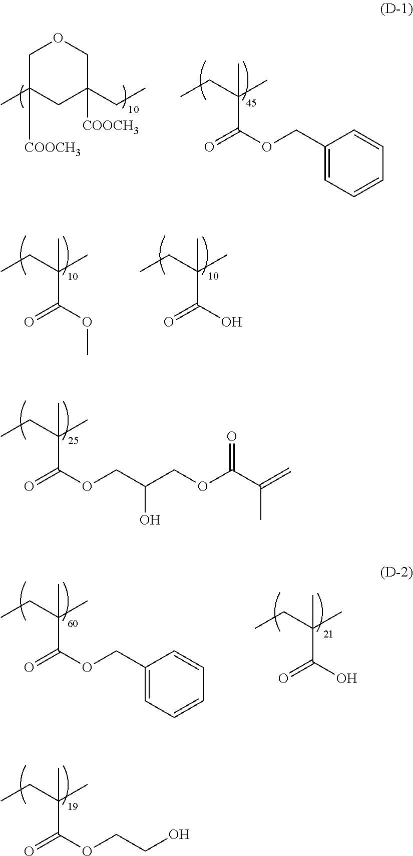

(Polymerizable Compound)

E-1: KAYARAD DPHA (manufactured by Nippon Kayaku Co., Ltd.)

E-2: ARONIX M-305 (manufactured by TOAGOSEI CO., LTD.)

E-3: NK ESTER A-TMMT (manufactured by Shin-Nakamura Chemical Co., Ltd.)

E-4: KAYARAD RP-1040 (manufactured by Nippon Kayaku Co., Ltd.)

E-5: ARONIX TO-2349 (manufactured by TOAGOSEI CO., LTD.)

(Photopolymerization Initiator)

F-1: compound having the following structure (oxime-based photopolymerization initiator)

F-2: compound having the following structure (oxime-based photopolymerization initiator)

F-3: compound having the following structure (oxime-based photopolymerization initiator)

F-4: compound having the following structure (alkylphenone-based photopolymerization initiator)

F-5: compound having the following structure (oxime-based photopolymerization initiator)

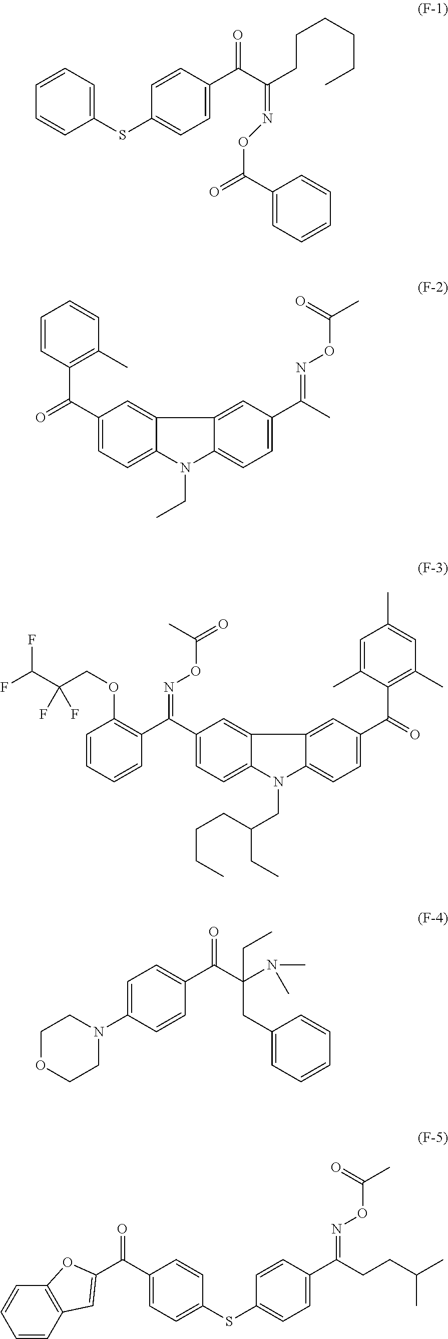 [SMILES: CCCCCC/C(=N/OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21 ; CCCCC(CC)Cn1c2ccc(C(=O)c3c(C)cc(C)cc3C)cc2c2cc(/C(=N\OC(C)=O)c3ccccc3OCC(F)(F)C(F)F)ccc21 ; CCC(Cc1ccccc1)(C(=O)c1ccc(N2CCOCC2)cc1)N(C)C ; CC(=O)O/N=C(\CCC(C)C)c1ccc(Sc2ccc(C(=O)c3cc4ccccc4o3)cc2)cc1]

(Surfactant)

W-1: 1% by mass of PGMEA solution of the following mixture (Mw=14000; in the following formula, “%” representing the proportion of a repeating unit is mol %)

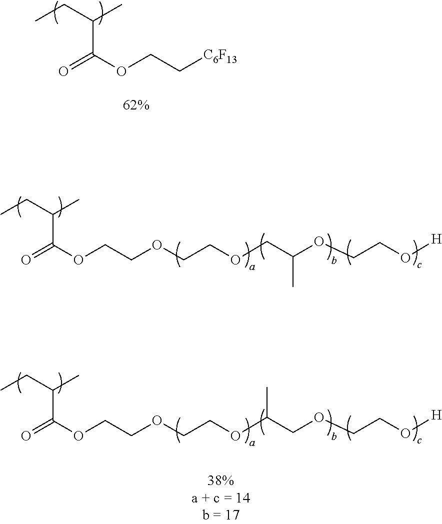

(Polymerization Inhibitor)

G-1: p-methoxyphenol

[Solvent]

S-1: propylene glycol monomethyl ether acetate (PGMEA)

S-2: cyclohexanone

(Dye)

Dye 1: dye having the following structure (synthesized in the same manner as in Synthesis Example C-48 of paragraph No. 0468 of JP2016-102191A; in the following structural formula, i-Pr represents an isopropyl group)

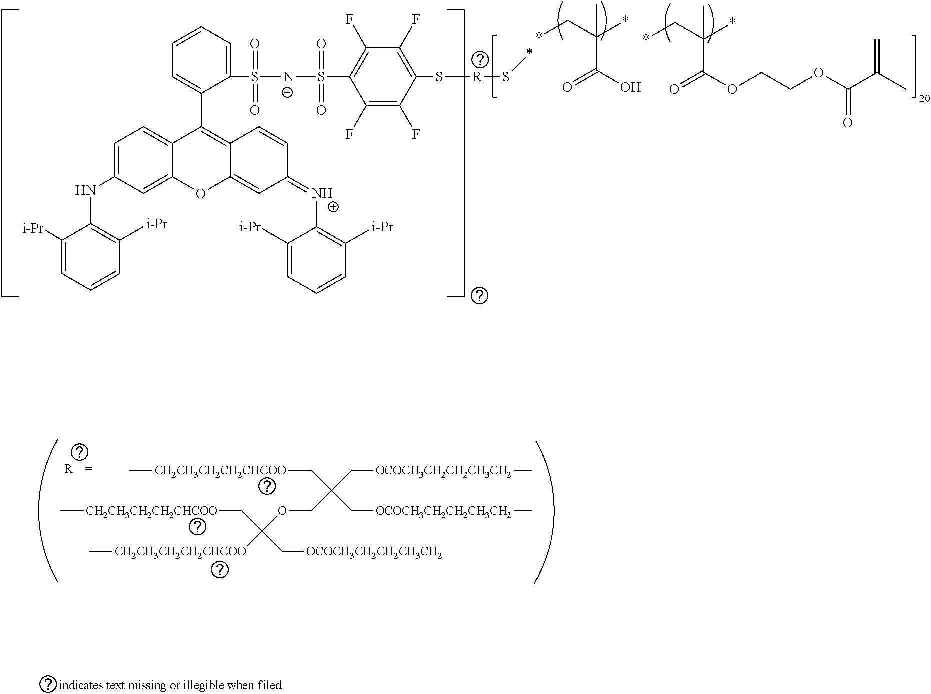

Dye 2: dye having the following structure (synthesized in the same manner as in paragraph No. of JP2014-237809A; in the following structural formula, Et represents an ethyl group)

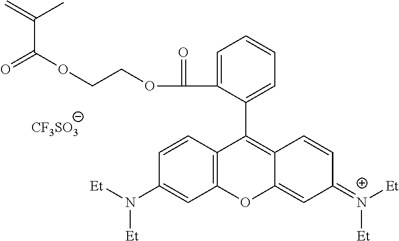

Dye 3: dye having the following structure (synthesized in the same manner as in paragraph No. 0419 of JP2014-237809A; in the following structural formula, Et represents an ethyl group)

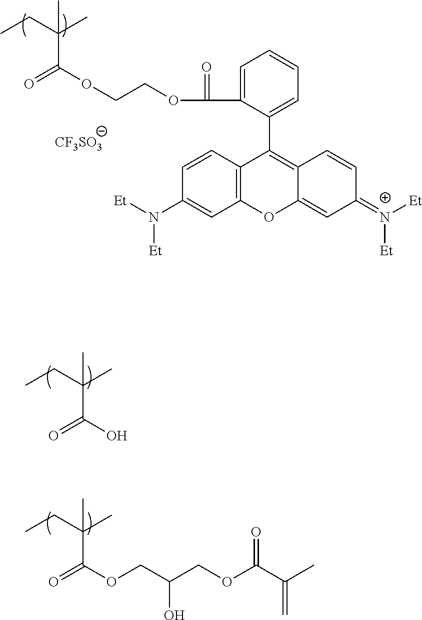

(Additive)

Additive 1: potassium bis(trifluoromethanesulfonyl)imide

<Evaluation of Storage Stability>

The viscosity of the coloring composition obtained as described above was measured by “RE-85L” manufactured by TOM SANGYO CO., LTD. After that, the coloring composition was left to stand under the conditions of 45° C. and 3 days, and then the viscosity thereof was measured again. Storage stability was evaluated according to the following evaluation standard from a viscosity difference (AVis) before and after leaving to stand. It can be said that the smaller the numerical value of the viscosity difference (AVis), the better the storage stability. The viscosity of the coloring composition was measured in a state in which the temperature was adjusted to 25° C. The evaluation standard is as follows, and the evaluation results are shown in the tables below.

[Evaluation Standard]

A: AVis was 0.5 mPa·s or less.

B: AVis was more than 0.5 mPa·s and 2.0 mPa·s or less.

C: AVis was more than 2.0 mPa·s.

<Evaluation of Adhesiveness>

An 8-inch (20.32 cm) silicon wafer was coated with each coloring composition by a spin coating method so that the film thickness after post-baking was 0.5 μm. Next, the silicon wafer was pre-baked using a hot plate at 100° C. for 2 minutes. Next, using an i-ray stepper exposure device FPA-3000 i5+(manufactured by Canon Corporation), exposure was performed with an exposure amount of 200 mJ/cm² through a mask having a Bayer pattern in which a predetermined pixel (pattern) size was formed. As the mask, a mask having a Bayer pattern in which a pixel pattern is formed in a shape of 0.7 μm square, 0.8 μm square, 0.9 μm square, 1.0 μm square, 1.1 μm square, 1.2 μm square, 1.3 μm square, 1.4 μm square, 1.5 μm square, 1.7 μm square, 2.0 μm square, 3.0 μm square, 5.0 μm square, or 10.0 μm square was used.

Next, puddle development was performed at 23° C. for 60 seconds using a 0.3% by mass of tetramethylammonium hydroxide (TMAH) aqueous solution. Thereafter, rinsing was performed by a spin shower using pure water. Next, a pattern (pixel) was formed by heating (post-baking) at 200° C. for 5 minutes using a hot plate.

Using a high-resolution FEB measuring device (HITACHI CD-SEM) S9380II (manufactured by Hitachi High-Tech Corporation.), a pattern of 0.7 μm square, 0.8 μm square, 0.9 μm square, 1.0 μm square, 1.1 μm square, 1.2 μm square, 1.3 μm square, 1.4 μm square, 1.5 μm square, 1.7 μm square, 2.0 μm square, 3.0 μm square, 5.0 μm square, or 10.0 μm square was observed, and the minimum pattern size in which the pattern was formed without peeling was defined as a minimum contact line width. It means that the smaller the minimum contact line width, the better the adhesiveness is.

[Evaluation Standard]

A: minimum contact line width was 1.2 μm square or less.

B: minimum contact line width was more than 1.2 μm square and 1.3 μm square or less.

C: minimum contact line width was more than 1.3 μm square and 1.4 μm square or less.

D: minimum contact line width was more than 1.4 μm square and 1.6 μm square or less.

E: minimum contact line width was more than 1.6 μm square.

<Evaluation of Color Unevenness>

A 5 cm×5 cm glass substrate was coated with each coloring composition using a spin coater so that the thickness of a film after pre-baking was 0.6 μm, and pre-baking was performed at 100° C. for 120 seconds to obtain a color filter for evaluation of color unevenness. The brightness distribution of the obtained color filter was analyzed by the following method, and the color unevenness was evaluated based on the number of pixels having deviation from the average of ±8% or more. A method of measuring the brightness distribution will be described. The color filter for evaluation of color unevenness was installed between an observation lens and a light source of an optical microscope, and the light source irradiated light toward the observation lens. The transmitted light state was observed with an optical microscope MX-50 (manufactured by Olympus Corporation) equipped with a digital camera. The capturing of the surface of the color filter was performed for five arbitrarily selected regions. The brightness of the captured image (total number of pixels: 636,416) was digitized and stored as a density distribution of 256 gradations from 0 to 255. The brightness distribution was analyzed from this image, and the color unevenness was evaluated based on the number of pixels (value of the roughness) having deviation from the average of more than ±8%.

The evaluation standard is as follows.

A: value of the roughness was 3000 or less.

B: value of the roughness was more than 3000 and 6000 or less.

C: value of the roughness was 6000 or more

(Evaluation of Heat Resistance)

A 5 cm×5 cm glass substrate was coated with each coloring composition by a spin coating method so that the film thickness after post-baking was 0.5 μm. Next, the silicon wafer was pre-baked using a hot plate at 100° C. for 2 minutes.

Next, using an i-ray stepper exposure device FPA-3000 i5+(manufactured by Canon Corporation), the silicon wafer was exposed with light at a wavelength of 365 nm at an exposure amount of 500 mJ/cm² to produce a film.

Next, the glass substrate was placed on a horizontal rotary table of a spin-shower developing machine (DW-30 Type, manufactured by Chemitronics Co., Ltd.), and was subjected to a puddle development at 23° C. for 60 seconds using CD-2000 (manufactured by FUJIFILM Electronic Materials Co., Ltd.). Thereafter, while rotating the glass substrate by a rotation device at a rotation speed of 50 rpm the glass substrate was rinsed by supplying pure water from above the center of rotation in shower-like from an ejection nozzle, and then spray-dried to produce a film.

Next, the glass substrate on which the film was formed was placed on a hot plate at 230° C. and heated for 1 hour. Using a chromaticity meter MCPD-3000 (manufactured by OTSUKA ELECTRONICS Co., LTD.), the color difference (ΔE*ab value) of the film before and after heating was measured to evaluate the heat resistance. As the ΔE*ab value is smaller, the heat resistance is better. The evaluation was performed according to the following standards.

A: ΔE*ab value was 0 or more and less than 1.0.

B: ΔE*ab value was 1.0 or more and less than 1.5.

C: ΔE*ab value was 1.5 or more.

TABLE 13

| | Content of | | | | |
|---|---|---|---|---|---|
| | pigment in | | | | |
| | total solid | | | Color | |
| | content | Storage | Adhesive- | uneven- | Heat |
| | (mass %) | stability | ness | ness | resistance |
| Example-1 | 60% | B | B | B | A |
| Example-2 | 60% | A | B | A | A |
| Example-3 | 60% | A | B | A | A |
| Example-4 | 60% | A | A | A | A |
| Example-5 | 60% | B | B | B | B |
| Example-6 | 60% | B | A | B | A |
| Example-7 | 60% | B | A | B | A |
| Example-8 | 60% | B | B | B | A |
| Example-9 | 60% | A | A | A | A |
| Example-10 | 60% | A | A | A | A |
| Example-11 | 60% | A | A | A | A |
| Example-12 | 60% | A | A | A | A |
| Example-13 | 60% | A | A | A | A |
| Example-14 | 60% | A | A | A | A |
| Example-15 | 60% | A | A | A | A |
| Example-16 | 60% | A | A | A | A |
| Example-17 | 60% | A | A | A | A |
| Example-18 | 60% | A | A | A | A |
| Example-19 | 60% | A | A | A | A |
| Example-20 | 60% | B | A | B | A |
| Example-21 | 60% | A | B | A | A |
| Example-22 | 60% | A | A | B | A |
| Example-23 | 60% | A | A | A | A |
| Example-24 | 60% | A | A | A | A |
| Example-25 | 60% | A | A | A | A |
| Example-26 | 60% | A | A | A | A |
| Example-27 | 60% | A | A | A | A |
| Example-28 | 60% | A | A | A | A |
| Example-29 | 60% | A | A | B | A |
| Example-30 | 60% | A | A | B | A |
| Example-31 | 60% | A | A | B | A |
| Example-32 | 60% | A | A | B | A |
| Example-33 | 60% | A | A | B | A |
| Example-34 | 60% | A | A | B | A |
| Example-35 | 60% | A | B | B | A |
| Example-36 | 60% | A | B | B | A |
| Example-37 | 60% | A | A | B | A |
| Example-38 | 60% | A | A | B | A |
| Example-39 | 60% | A | A | B | A |
| Example-40 | 60% | A | A | A | A |

TABLE 14

| | Content of | | | | |
|---|---|---|---|---|---|
| | pigment in | | | | |
| | total solid | | | Color | |
| | content | Storage | Adhesive- | uneven- | Heat |
| | (mass %) | stability | ness | ness | resistance |
| Example-41 | 60% | A | A | A | A |
| Example-42 | 60% | A | A | A | A |
| Example-43 | 60% | A | A | A | A |
| Example-44 | 60% | A | A | A | A |
| Example-45 | 60% | A | A | A | A |
| Example-46 | 60% | B | A | B | A |
| Example-47 | 58% | A | B | A | A |
| Example-48 | 56% | A | B | A | A |
| Example-49 | 53% | A | C | A | A |
| Example-50 | 60% | A | A | A | A |
| Example-51 | 60% | A | A | A | A |
| Example-52 | 60% | A | A | A | A |
| Example-53 | 60% | A | A | A | A |
| Example-54 | 60% | A | A | A | A |
| Example-55 | 60% | A | A | A | A |
| Example-56 | 60% | A | A | A | A |
| Example-57 | 60% | A | B | A | B |
| Example-58 | 60% | A | A | A | A |
| Example-59 | 60% | A | A | A | A |
| Example-60 | 56% | A | C | A | B |
| Example-61 | 60% | A | A | A | A |
| Example-62 | 60% | A | A | A | A |
| Example-63 | 60% | A | A | A | A |
| Example-64 | 60% | A | A | A | A |
| Example-65 | 60% | A | A | A | A |
| Example-66 | 60% | A | B | A | B |
| Example-67 | 60% | A | A | A | A |
| Comparative | 56% | A | E | B | C |
| example-1 | | | | | |
| Comparative | 56% | A | D | B | C |
| example-2 | | | | | |

TABLE 15

| | Content of | | | | |
|---|---|---|---|---|---|
| | pigment in | | | | |
| | total solid | | | Color | |
| | content | Storage | Adhesive- | uneven- | Heat |
| | (mass %) | stability | ness | ness | resistance |
| Example-101 | 60% | B | B | B | A |
| Example-102 | 60% | B | B | C | A |

TABLE 16

| | Content of | | | | |
|---|---|---|---|---|---|
| | pigment in | | | | |
| | total solid | | | Color | |
| | content | Storage | Adhesive- | uneven- | Heat |
| | (mass %) | stability | ness | ness | resistance |
| Example-201 | 60% | B | A | B | A |
| Example-202 | 60% | B | A | C | A |

TABLE 17

| | Content of | | | | |
|---|---|---|---|---|---|
| | pigment in | | | | |
| | total solid | | | Color | |
| | content | Storage | Adhesive- | uneven- | Heat |
| | (mass %) | stability | ness | ness | resistance |
| Example-203 | 50% | B | C | B | A |
| Example-204 | 50% | B | C | B | A |
| Example-205 | 50% | B | C | B | A |

As shown in the above tables, Examples were excellent in evaluation of storage stability and adhesiveness.

The same results were obtained by adding 0.2 parts by mass of 7,7,8,8,-tetracyanoquinodimethane to the coloring composition of Example-11.

In the coloring compositions of Examples-203, 204, and 205, even in a case where the additive 1 was changed to the same amount of lithium bis(trifluoromethanesulfonyl)imide, potassium N,N-bis(nonafluorobutanesulfonyl)imide, cesium tris(trifluoromethanesulfonyl)methide, or tetrakis(pentafluofluorophenyl) lithium borate, the same results of these examples were obtained.

### Example-301

A silicon wafer was coated with a Green composition by a spin coating method so that the film thickness after post-baking was 1.0 μm. Next, the silicon wafer was heated using a hot plate at 100° C. for 2 minutes. Next, using an i-ray stepper exposure device FPA-3000 i5+ (manufactured by Canon Corporation), exposure was performed with light having a wavelength of 365 nm and an exposure amount of 1000 mJ/cm² through a mask having a dot pattern of 2 μm square. Next, puddle development was performed at 23° C. for 60 seconds using a 0.3% by mass of tetramethylammonium hydroxide (TMAH) aqueous solution. Next, the coating film was rinsed by spin showering and was cleaned with pure water. Next, the Green composition was patterned by heating (post-baking) at 200° C. for 5 minutes using a hot plate. Likewise, a Red composition and a Blue composition were sequentially patterned to form red, green, and blue colored patterns (Bayer pattern).

As the Green composition, the coloring composition of Example 11 was used. As the Red composition, the coloring composition of Example 101 was used. As the Blue composition, the coloring composition of Example 201 was used.

The Bayer pattern refers to a pattern, as disclosed in the specification of U.S. Pat. No. 3,971,065A, in which a 2×2 array of color filter element having one Red element, two Green elements, and one Blue element is repeated.

The obtained color filter was incorporated into a solid-state imaging element according to a known method. The solid-state imaging element had a suitable image recognition ability.

What is claimed is:

1. A coloring composition for a solid-state imaging element, comprising:
a pigment;
a compound having a triazine ring;
a polymerizable compound; and
a photopolymerization initiator,
wherein the pigment is contained in an amount of 50% by mass or more in a total solid content of the coloring composition, and
the compound having a triazine ring includes at least one group selected from an acid group or a basic group, and a maximum value of a molar absorption coefficient of the compound having a triazine ring in a wavelength range of 400 to 700 nm is 3000 L·mol⁻¹·cm⁻¹ or less.

2. The coloring composition according to claim 1,
wherein the compound having a triazine ring includes two or more triazine rings in one molecule.

3. The coloring composition according to claim 1,
wherein the compound having a triazine ring includes a group represented by Formula (C1), 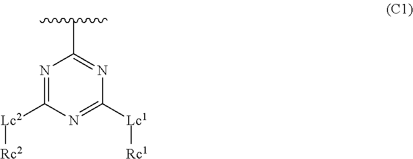
in the formula, a wavy line represents a bonding site, Lc¹ and Lc² each independently represent a single bond or a linking group, Rc¹ and Rc² each independently represent a substituent, and at least one of Rc¹ or Rc² represents an acid group or a basic group.

4. The coloring composition according to claim 3,
wherein the group represented by Formula (C1) is a group represented by Formula (C2), 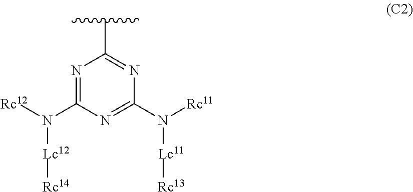
in the formula, a wavy line represents a bonding site, Lc¹¹ and Lc¹² each independently represent a single bond or a linking group, Rc¹¹ and Rc¹² each independently represent a hydrogen atom or a substituent, Rc¹³ and Rc¹⁴ each independently represent a substituent, and at least one of Rc¹³ or Rc¹⁴ represents an acid group or a basic group.

5. The coloring composition according to claim 3,
wherein the group represented by Formula (C1) is a group represented by Formula (C3), 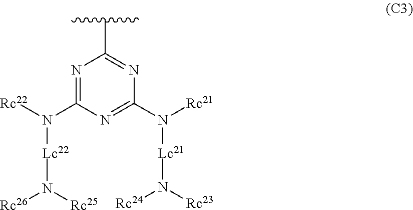
in the formula, a wavy line represents a bonding site, Lc²¹ and Lc²² each independently represent a single bond or a linking group, Rc²¹ and Rc²² each independently represent a hydrogen atom or a substituent, Rc²³ to Rc²⁶ each independently represent a hydrogen atom or a substituent, Rc²³ and Rc²⁴ may be bonded to each other through a divalent group to form a ring, and Rc²⁵ and Rc²⁶ may be bonded to each other through a divalent group to form a ring.

6. The coloring composition according to claim 1,
wherein the pigment is an organic pigment.

7. The coloring composition according to claim 1,
wherein the pigment is a chromatic pigment.

8. The coloring composition according to claim 1,
wherein the pigment includes a phthalocyanine pigment.

9. The coloring composition according to claim 1,
wherein the pigment includes a green pigment.

10. The coloring composition according to claim 1,
wherein the photopolymerization initiator includes an oxime compound.

11. The coloring composition according to claim 1, further comprising:
an alkali-soluble resin.

12. The coloring composition according to claim 1, further comprising:
a resin having an aromatic carboxyl group.

13. A film obtained from the coloring composition according to claim 1.

14. A color filter comprising:
the film according to claim 13.

15. A solid-state imaging element comprising:
the film according to claim 13.
